(19) United States
(12) Patent Application Publication
(10) Pub. No.: US 2017/0348165 A1
(43) Pub. Date: Dec. 7, 2017

(54) THREE-DIMENSIONAL MATERIALS

(71) Applicant: The Procter & Gamble Company, Cincinnati, OH (US)

(72) Inventor: Adrien GRENIER, Frankfurt Am Main (DE)

(21) Appl. No.: 15/453,955

(22) Filed: Mar. 9, 2017

Related U.S. Application Data
(60) Provisional application No. 62/306,881, filed on Mar. 11, 2016.

(51) Int. Cl. A61F 13/511; A61F 13/536; A61F 13/539; A61F 13/51; A61F 13/513

(52) U.S. Cl. CPC A61F 13/51104; A61F 13/539; A61F 13/52; A61F 13/51121; A61F 13/536; A61F 2013/51019; A61F 2013/51361

(57) ABSTRACT

An absorbent article comprises a laminate, a backsheet, and an absorbent core disposed at least partially between the laminate and the backsheet. The laminate comprises a nonwoven, non-apertured topsheet and a nonwoven, non-apertured acquisition layer. The topsheet is less hydrophilic than the acquisition layer. The laminate comprises a generally planar first region and a plurality of discrete integral second regions formed in the topsheet and the acquisition layer to nest portions of the topsheet and the acquisition layer. The plurality of discrete integral second regions each comprise a wearer-facing surface. A non-uniform interruption is defined in the topsheet in each of at least some of the plurality of discrete integral second regions. Portions of the acquisition layer form portions of the wearer-facing surface in the non-uniform interruption in the at least some of the discrete integral second regions.

### CROSS REFERENCE TO RELATED APPLICATION

This application claims the benefit, under 35 U.S.C. §119(e), to U.S. Provisional Patent Application No. 62/306,881, filed on Mar. 11, 2016, which is herein incorporated by reference in its entirety.

### FIELD

The present disclosure is directed to three-dimensional materials. The present disclosure is also directed to absorbent articles comprising three-dimensional nonwoven materials having reduced rewet and increased acquisition.

### BACKGROUND

Disposable absorbent articles, such as disposable diapers, pants, adult incontinence products, and feminine products, for example, are widely used, and much effort has been made to improve the effectiveness and functionality of these absorbent articles. In general, such absorbent articles have a fluid permeable topsheet, a fluid impermeable backsheet, and an absorbent core disposed at least partially intermediate the topsheet and the backsheet. The absorbent articles sometimes comprise an acquisition layer and optionally a distribution layer, along with other components. The acquisition layer is configured to receive bodily exudates (e.g., bowel movements (“BM”), runny BM, urine, and/or menses) from the topsheet in order to wick the bodily exudates towards the absorbent core. Then, if provided, the distribution layer may receive the bodily exudates from the acquisition layer and distribute and transfer them to the absorbent core in order to make the absorbent core more efficient.

In recent years, three-dimensional topsheet structures have been developed. These structures may have hydrophilic topsheets in order to allow for the bodily exudates to be quickly absorbed into and through the topsheets. Typically, it is desired to have a capillary gradient between the topsheet and the acquisition layer to achieve good dryness. However, hydrophilic topsheets tend to retain some of the bodily exudates, which may limit dryness performance and may cause rewet. Acquisition layers are typically hydrophilic to allow them to wick the bodily exudates away from the topsheet and into the absorbent core. In some instances, especially with a hydrophilic topsheet, however, the acquisition layer may cause rewet in the topsheet. There is a need to develop better three-dimensional topsheet structures and acquisition layers to reduce rewet in the topsheets and increase bodily exudate absorbency.

Further, planar apertured topsheets sometimes used hydrophobic materials, such as films or nonwoven materials. The issue with these planar apertured hydrophobic topsheets is that the apertures have to be fairly large to allow bodily exudates to pass through them. In some instances, the large apertures may cause softness issues, skin red-marking, and rewet (through the apertures). As such, planar apertured hydrophobic topsheets need to be improved to create better softness, less skin red-marking and reduced rewet, while still providing adequate acquisition speed.

### SUMMARY

The present disclosure is directed, at least in part, to an absorbent article that reduces rewet and increases acquisition speed in a topsheet. The absorbent article comprises a topsheet that is nested or otherwise combined with an acquisition layer, thereby forming a three-dimensional topsheet/acquisition layer laminate. The absorbent article may comprise a backsheet and an absorbent core positioned at least partially intermediate the three-dimensional topsheet/acquisition layer laminate and the backsheet. The three-dimensional topsheet/acquisition layer laminate may have a planar first region and a plurality of discrete integral second regions. The plurality of discrete integral second regions may extend towards the absorbent core. The topsheet may be hydrophobic and the acquisition layer may be hydrophilic. In other instances, the topsheet may be less hydrophilic than the acquisition layer. The topsheet may comprise or be formed of cotton. The cotton may be hydrophobic. Both the topsheet and the acquisition layer may be non-apertured. Non-apertured means no uniform, predetermined, non-random apertures were formed in the topsheet (like an apertured topsheet). The topsheet, however, may have non-uniform, non-predetermined, and random rips, tears, interruptions, or areas having low density fibers (hereafter “non-uniform interruptions”) in the discrete integral second regions. The non-uniform interruptions are not pores or voids formed during production of a nonwoven material. In fact, any pores or voids that have a major axis less than 500 microns are not considered non-uniform interruptions. The non-uniform interruptions are larger in area than the average pore size in the nonwoven material. As an example, a non-uniform interruption may have an area at least 2 times larger than the average pore or void area in a nonwoven material. These non-uniform interruptions may be formed during the creation of the three-dimensional structure when the topsheet is nested with or combined with the acquisition layer. The non-uniform interruptions may be ruptures or stretched areas (low fiber density) in the topsheet. The non-uniform interruptions in the topsheet allow areas of the acquisition layer to form portions of the wearer-facing surface of the absorbent article. By having areas of the acquisition layer forming portions of the wearer-facing surface, the hydrophilic acquisition layer is able to directly contact bodily exudates and quickly wick the bodily exudates into the absorbent core or a distribution material positioned over the absorbent core without restriction from the hydrophobic topsheet, or the less hydrophilic topsheet. Stated another way, the wearer-facing surface of the absorbent article is formed by portions of the topsheet and portions of the acquisition layer. The hydrophobic topsheet or less hydrophilic topsheet also aids in directing bodily exudates to the exposed acquisition layer areas since it restrict the flow of bodily exudates therethrough. This provides for superior absorbency of bodily exudates and significantly reduced rewet and increased acquisition speed. In the case of a hydrophobic topsheet, a capillary gradient is not required between the topsheet and the acquisition layer in that the topsheet does not need to be dewatered.

Owing to the fact that the non-uniform interruptions are positioned within the discrete integral second regions, and the fact that only the generally planar first region contacts the wearer, better softness and reduced skin red-marking may be achieved comprise to a planar apertured topsheet. Furthermore, the non-uniform interruptions may have a tendency to at least partially close up after insults of bodily exudates pass through them and pressure is applied by the wearer, thereby leading to reduced rewet. Additionally, the topsheet/acquisition layer laminate has increased breathability and a cushiony softness owning to the three-dimensional structure, even with a hydrophobic topsheet, or a topsheet that is less hydrophilic than the acquisition layer.

In a form, the present disclosure is directed in part, to an absorbent article comprising a laminate, a backsheet, and an absorbent core disposed at least partially between the laminate and the backsheet. The laminate comprises a nonwoven, non-apertured topsheet and a nonwoven, non-apertured acquisition layer. The topsheet is less hydrophilic than the acquisition layer. The laminate comprises a generally planar first region and a plurality of discrete integral second regions formed in the topsheet and the acquisition layer to nest portions of the topsheet and the acquisition layer. The plurality of discrete integral second regions each comprise a wearer-facing surface. A non-uniform interruption is defined in the topsheet in each of at least some of the plurality of discrete integral second regions. Portions of the acquisition layer form portions of the wearer-facing surface in the non-uniform interruption in the at least some of the discrete integral second regions.

In a form, the present disclosure is directed in part, to an absorbent article comprising a laminate, a backsheet, and an absorbent core disposed at least partially between the laminate and the backsheet. The laminate comprises a nonwoven, non-apertured topsheet and a nonwoven, non-apertured acquisition layer. The topsheet is less hydrophilic than the acquisition layer. The laminate comprises a generally planar continuous land area and a plurality of three-dimensional deformations. At least some of the plurality of three-dimensional deformations are surrounded by at least some of the generally planar continuous land area. The plurality of three-dimensional deformations each comprise a wearer-facing surface. A non-uniform interruption is defined in the topsheet in each of at least some of the plurality of three-dimensional deformations. Portions of the acquisition layer form portions of the wearer-facing surface in the non-uniform interruption in the at least some of the plurality of three-dimensional deformations.

In a form, the present disclosure is directed in part, to an absorbent article comprising a laminate, a backsheet, and an absorbent core disposed at least partially between the laminate and the backsheet. The laminate comprises a film, non-apertured topsheet and a nonwoven, non-apertured secondary topsheet. The topsheet is hydrophobic than the secondary topsheet is hydrophilic. The laminate comprises a generally planar continuous land area and a plurality of three-dimensional deformations. At least some of the plurality of three-dimensional deformations are surrounded by at least some of the generally planar continuous land area. The plurality of three-dimensional deformations each comprise a wearer-facing surface. A non-uniform interruption is defined in the topsheet in each of at least some of the plurality of three-dimensional deformations. Portions of the secondary topsheet form portions of the wearer-facing surface in the non-uniform interruption in the at least some of the plurality of three-dimensional deformations.

### BRIEF DESCRIPTION OF THE DRAWINGS

The above-mentioned and other features and advantages of the present disclosure, and the manner of attaining them, will become more apparent and the disclosure itself will be better understood by reference to the following description of non-limiting forms of the disclosure taken in conjunction with the accompanying drawings, wherein:

FIG. 1 is a photomicrograph showing the end view of a prior art tuft;

FIG. 2 is a schematic end view of a prior art tuft after it has been subjected to compression;

FIG. 3 is a photomicrograph of the end of a prior art nonwoven web showing a plurality of collapsed tufts;

FIG. 4 is a schematic side view of a prior art conical-shaped structure before and after it has been subjected to compression;

FIG. 5 is a plan view photomicrograph showing one side of the nonwoven material having three-dimensional deformations formed therein, with the protrusions oriented upward;

FIG. 6 is a plan view photomicrograph showing the other side of a nonwoven material similar to that shown in FIG. 5, with the openings in the nonwoven facing upward;

FIG. 7 is a Micro CT scan image showing a perspective view of a protrusion in a single layer nonwoven material;

FIG. 8 is a Micro CT scan image showing a side of a protrusion in a single layer nonwoven material;

FIG. 9 is a Micro CT scan image showing a perspective view of a deformation with the opening facing upward in a single layer nonwoven material;

FIG. 10 is a perspective view of a deformation in a two layer nonwoven material with the opening facing upward;

FIG. 11 is a photomicrograph of a cross-section taken along the transverse axis of a deformation showing one example of a multi-layer nonwoven material having a three-dimensional deformation in the form of a protrusion on one side of the material that provides a wide opening on the other side of the material, with the opening facing upward;

FIG. 12 is a schematic view of the protrusion shown in FIG. 11;

FIG. 13 is a plan view photomicrograph from the protrusion side of a material after it has been subjected to compression showing the high fiber concentration region around the perimeter of the protrusion;

FIG. 14 is a photomicrograph of the cross-section of a protrusion taken along the transverse axis of the protrusion showing the protrusion after it has been subjected to compression;

FIG. 14A is a photomicrograph of the cross-section of a protrusion taken along the transverse axis of the protrusion showing the protrusion after it has been subjected to compression;

FIG. 15A is a cross-sectional view taken along the transverse axis of a deformation of one form of a multi-layer nonwoven web shown with the base opening facing upward;

FIG. 15B is a cross-sectional view taken along the transverse axis of a deformation of an alternative form of a multi-layer nonwoven web shown with the base opening facing upward;

FIG. 15C is a cross-sectional view taken along the transverse axis of a deformation of an alternative form of a multi-layer nonwoven web shown with the base opening facing upward;

FIG. 15D is a cross-sectional view taken along the transverse axis of a deformation of an alternative form of a multi-layer nonwoven web shown with the base opening facing upward;

FIG. 15E is a cross-sectional view taken along the transverse axis of a deformation of an alternative form of a multi-layer nonwoven web shown with the base opening facing upward;

FIG. 15F is a cross-sectional view taken along the transverse axis of a deformation of an alternative form of a multi-layer nonwoven web shown with the base opening facing upward;

FIG. 16 is a plan view photomicrograph of a nonwoven web with the protrusions oriented upward showing the concentration of fibers in one layer of a two layer structure;

FIG. 17 is a perspective view photomicrograph showing the reduced fiber concentration in the side walls of the protrusions in a layer similar to that shown in FIG. 16;

FIG. 18 is a plan view photomicrograph of a nonwoven web with the protrusions oriented upward showing the reduced concentration of fibers in the cap of a protrusion in the other layer (i.e. vs. the layer shown in FIG. 16) of a two layer structure;

FIG. 19 is a perspective view photomicrograph showing the decreased fiber concentration in the side walls of the protrusions in a layer similar to that shown in FIG. 18;

FIG. 19A is a Micro CT scan image showing the side of a protrusion in a single layer of nonwoven material with the protrusion oriented downward;

FIG. 19B is a Micro CT scan plan view image showing the base opening of a deformation in a single layer of nonwoven material;

FIG. 20 is a perspective view photomicrograph of one layer of a multiple layer nonwoven material on the surface of a forming roll showing the “hanging chads” that can be formed in one of the layers when some nonwoven precursor web materials are used;

FIG. 21 is a perspective view of one example of an apparatus for forming the nonwoven material described herein;

FIG. 22 is an enlarged perspective view of a portion of the male roll shown in FIG. 21;

FIG. 22A is a schematic side view of a male element with tapered side walls;

FIG. 22B is a schematic side view of a male element with undercut side walls;

FIG. 22C is an enlarged perspective view of a portion of a male roll having an alternative configuration;

FIG. 22D is a schematic side view of a male element with a rounded top;

FIG. 23 is an enlarged perspective view showing the nip between the rolls shown in FIG. 21;

FIG. 24 is a schematic perspective view of one version of a method of making nonwoven materials having deformations therein where two precursor materials are used, one of which is a continuous web and the other of which is in the form of discrete pieces;

FIG. 24A is a schematic side view of an apparatus for forming the nonwoven material in which the web wraps around one of the rolls before and after passing through the nip between the rolls;

FIG. 25 is an absorbent article in the form of a diaper comprising an exemplary topsheet/acquisition layer composite structure wherein the length of the acquisition layer is less that the length of the topsheet with some layers partially removed;

FIG. 26 is one transverse cross-section of the diaper of FIG. 25 taken along line 26-26;

FIG. 27 is an alternative transverse cross-section of the diaper of FIG. 25;

FIGS. 28-33 are example cross-sectional illustrations of three-dimensional features formed in topsheets and acquisition layers and having non-uniform interruptions;

FIG. 34 is a microCT of a three-dimensional feature formed in a topsheet and acquisition layer and having a non-uniform interruption;

FIG. 35 is a cross-sectional example illustration of a three-dimensional deformation with two non-uniform interruptions;

FIG. 36 is a top view illustration of a wearer-facing surface of a nonwoven material laminate with a generally planar continuous land area and a plurality of three-dimensional deformations;

FIG. 37 is a cross-sectional view illustration taken about line 37-37 of FIG. 36;

FIG. 38 is a cross-section example illustration of another three-dimensional deformation;

FIG. 39 shows an equipment assembly used in the Flat Acquisition Method;

FIG. 40 shows an equipment assembly used in the Post Acquisition Collagen Rewet Method; and

FIG. 41 shows an equipment assembly used in the Fixed Height Frit Absorption (FHFA) Test Methods.

### DETAILED DESCRIPTION

Various non-limiting forms of the present disclosure will now be described to provide an overall understanding of the principles of the structure, function, manufacture, and use of the three-dimensional materials having apertures disclosed herein. One or more examples of these non-limiting forms are illustrated in the accompanying drawings. Those of ordinary skill in the art will understand that the three-dimensional materials having apertures described herein and illustrated in the accompanying drawings are non-limiting example forms and that the scope of the various non-limiting forms of the present disclosure are defined solely by the claims. The features illustrated or described in connection with one non-limiting form may be combined with the features of other non-limiting forms. Such modifications and variations are intended to be included within the scope of the present disclosure.

### I. Definitions

The term “absorbent article” includes disposable articles such as sanitary napkins, panty liners, tampons, interlabial devices, wound dressings, pants, diapers, adult incontinence articles, wipes, and the like. At least some of such absorbent articles are intended for the absorption of body liquids, such as menses or blood, vaginal discharges, urine, and feces. Wipes may be used to absorb body liquids, or may be used for other purposes, such as for cleaning surfaces. Various absorbent articles described above will typically comprise a liquid pervious topsheet, a liquid impervious backsheet joined to the topsheet, and an absorbent core between the topsheet and backsheet. The nonwoven material described herein can comprise at least part of other articles such as scouring pads, wet or dry-mop pads (such as SWIFFER® pads), and the like.

The term “absorbent core”, as used herein, refers to the component of the absorbent article that is primarily responsible for storing liquids. As such, the absorbent core typically does not include the topsheet or backsheet of the absorbent article.

The term “aperture”, as used herein, refers to a regular or substantially regularly-shaped, predetermined hole that is intentionally formed and extends completely through a web or structure (that is, a through hole). The apertures can either be punched cleanly through the web so that the material surrounding the aperture lies in the same plane as the web prior to the formation of the aperture (a “two dimensional” aperture), or the holes can be formed such that at least some of the material surrounding the opening is pushed out of the plane of the web. In the latter case, the apertures may resemble a depression with an aperture therein, and may be referred to herein as a “three dimensional” aperture, a subset of apertures.

The term “component” of an absorbent article, as used herein, refers to an individual constituent of an absorbent article, such as a topsheet, acquisition layer, liquid handling layer, absorbent core or layers of absorbent cores, backsheets, and barriers such as barrier layers and barrier cuffs.

The term “cross-machine direction” or “CD” means the path that is perpendicular to the machine direction in the plane of the web.

The term “deformable material”, as used herein, is a material which is capable of changing its shape or density in response to applied stresses or strains.

The term “discrete”, as used herein, means distinct or unconnected. When the term “discrete” is used relative to forming elements on a forming member, it is meant that the distal (or radially outwardmost) ends of the forming elements are distinct or unconnected in all directions, including in the machine and cross-machine directions (even though bases of the forming elements may be formed into the same surface of a roll, for example).

The term “disposable” is used herein to describe absorbent articles and other products which are not intended to be laundered or otherwise restored or reused as an absorbent article or product (i.e., they are intended to be discarded after use and, preferably, to be recycled, composted or otherwise disposed of in an environmentally compatible manner).

The term “forming elements”, as used herein, refers to any elements on the surface of a forming member that are capable of deforming a web.

The term “integral”, as used herein as in “integral extension” when used to describe the protrusions, refers to fibers of the protrusions having originated from the fibers of the precursor web(s). Thus, as used herein, “integral” is to be distinguished from fibers introduced to or added to a separate precursor web for the purpose of making the protrusions.

The term “joined to” encompasses configurations in which an element is directly secured to another element by affixing the element directly to the other element; configurations in which the element is indirectly secured to the other element by affixing the element to intermediate member(s) which in turn are affixed to the other element; and configurations in which one element is integral with another element, i.e., one element is essentially part of the other element. The term “joined to” encompasses configurations in which an element is secured to another element at selected locations, as well as configurations in which an element is completely secured to another element across the entire surface of one of the elements. The term “joined to” includes any known manner in which elements can be secured including, but not limited to mechanical entanglement.

The term “machine direction” or “MD” means the path that material, such as a web, follows through a manufacturing process.

The term “macroscopic”, as used herein, refers to structural features or elements that are readily visible and distinctly discernable to a human having 20/20 vision when the perpendicular distance between the viewer's eye and the web is about 12 inches (30 cm). Conversely, the term “microscopic” refers to such features that are not readily visible and distinctly discernable under such conditions.

The term “mechanically deforming”, as used herein, refers to processes in which a mechanical force is exerted upon a material in order to permanently deform the material.

The term “permanently deformed”, as used herein, refers to the state of a deformable material whose shape or density has been permanently altered in response to applied stresses or strains.

The terms “SELF” and “SELF'ing”, refer to Procter & Gamble technology in which SELF stands for Structural Elastic Like Film. While the process was originally developed for deforming polymer film to have beneficial structural characteristics, it has been found that the SELF'ing process can be used to produce beneficial structures in other materials. Processes, apparatuses, and patterns produced via SELF are illustrated and described in U.S. Pat. Nos.: 5,518,801; 5,691,035; 5,723,087; 5,891,544; 5,916,663; 6,027,483; and 7,527,615 B2.

The term “tuft”, as used herein, refers to a particular type of feature that may be formed from fibers in a nonwoven web. Tufts may have a tunnel-like configuration which may be open at both of their ends.

The term “web” is used herein to refer to a material whose primary dimension is X-Y, i.e., along its length (or longitudinal direction) and width (or transverse direction). It should be understood that the term “web” is not necessarily limited to single layers or sheets of material. Thus the web can comprise laminates or combinations of several sheets of the requisite type of materials.

The term “Z-dimension” refers to the dimension orthogonal to the length and width of the web or article. The Z-dimension usually corresponds to the thickness of the web or material. As used herein, the term “X-Y dimension” refers to the plane orthogonal to the thickness of the web or material. The X-Y dimension usually corresponds to the length and width, respectively, of the web or material.

The term “average diameter”, refers to an arithmetic mean diameter of fibers calculated from the fiber diameter, which is measured by the Fiber Diameter and Denier Test set forth below. Number-average diameter of fibers is calculated by the Fiber Diameter Calculations set forth below. The number-average diameter of fibers is set forth in microns.

As used herein “hydrophilic” and “hydrophobic” have meanings as well established in the art with respect to the contact angle of a referenced liquid on the surface of a material. Thus, a material having a liquid contact angle of greater than about 90 degrees is considered hydrophobic, and a material having a liquid contact angle of less than about 90 degrees is considered hydrophilic. Compositions which are hydrophobic, will increase the contact angle of a referenced liquid on the surface of a material while compositions which are hydrophilic will decrease the contact angle of a referenced liquid on the surface of a material. Notwithstanding the foregoing, reference to relative hydrophobicity or hydrophilicity between a material and a composition, between two materials, and/or between two compositions, does not imply that the materials or compositions are hydrophobic or hydrophilic. For example, a composition may be more hydrophobic than a material. In such a case neither the composition nor the material may be hydrophobic; however, the contact angle exhibited by the composition is greater than that of the material. As another example, a composition may be more hydrophilic than a material. In such a case, neither the composition nor the material may be hydrophilic; however, the contact angle exhibited by the composition may be less than that exhibited by the material. The contact angle of material may be determined by the Contact Angle Method herein.

### II. Nonwoven Materials

The present disclosure is directed to nonwoven materials having discrete three-dimensional deformations, which deformations provide protrusions on one side of the material, and openings on the other side of the nonwoven materials. Methods of making the nonwoven materials are also disclosed. The nonwoven materials can be used in absorbent articles and other articles.

As used herein, the term “nonwoven” refers to a web or material having a structure of individual fibers or threads which are interlaid, but not in a repeating pattern as in a woven or knitted fabric, which latter types of fabrics do not typically have randomly oriented or substantially randomly-oriented fibers. Nonwoven webs will have a machine direction (MD) and a cross machine direction (CD) as is commonly known in the art of web manufacture. By “substantially randomly oriented” is meant that, due to processing conditions of the precursor web, there may be a higher amount of fibers oriented in the MD than the CD, or vice versa. For example, in spunbonding and meltblowing processes continuous strands of fibers are deposited on a support moving in the MD. Despite attempts to make the orientation of the fibers of the spunbond or meltblown nonwoven web truly “random,” usually a slightly higher percentage of fibers are oriented in the MD as opposed to the CD.

Nonwoven webs and materials are often incorporated into products, such as absorbent articles, at high manufacturing line speeds. Such manufacturing processes can apply compressive and shear forces on the nonwoven webs that may damage certain types of three-dimensional features that have been purposefully formed in such webs. In addition, in the event that the nonwoven material is incorporated into a product (such as a disposable diaper) that is made or packaged under compression, it becomes difficult to preserve the three-dimensional character of some types of prior three-dimensional features after the material is subjected to such compressive forces.

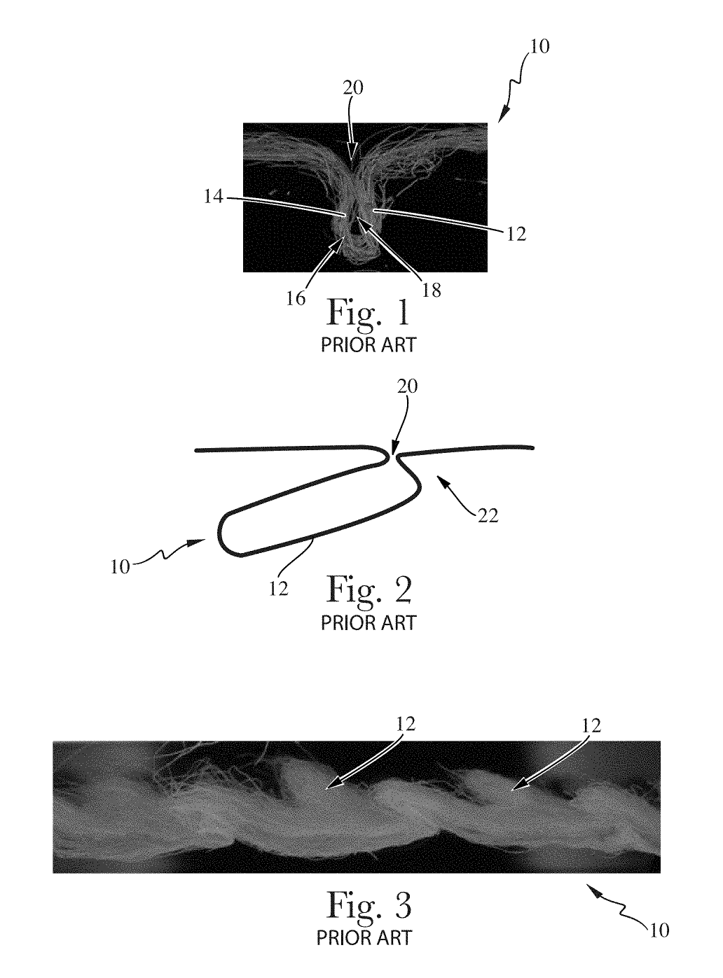

For instance, FIGS. 1 and 2 show an example of a prior art nonwoven material 10 with a tufted structure. The nonwoven material comprises tufts 12 formed from looped fibers 14 that form a tunnel-like structure having two ends 16. The tufts 12 extend outward from the plane of the nonwoven material in the Z-direction. The tunnel-like structure has a width that is substantially the same from one end of the tuft to the opposing end. Often, such tufted structures will have holes or openings 18 at both ends and an opening 20 at their base. Typically, the openings 18 at the ends of the tufts are at the machine direction (MD) ends of the tufts. The openings 18 at the ends of the tufts can be a result of the process used to form the tufts. If the tufts 12 are formed by forming elements in the form of teeth with a relatively small tip and vertical leading and trailing edges that form a sharp point, these leading and/or trailing edges may punch through the nonwoven web at least one of the ends of the tufts. As a result, openings 18 may be formed at one or both ends of the tufts 12.

While such a nonwoven material 10 provides well-defined tufts 12, the opening 20 at the base of the tuft structure can be relatively narrow and difficult to see with the naked eye. In addition, as shown in FIG. 2, the material of the tuft 12 surrounding this narrow base opening 20 may tend to form a hinge 22, or pivot point if forces are exerted on the tuft. If the nonwoven is compressed (such as in the Z-direction), in many cases, the tufts 12 can collapse to one side and close off the opening 20. Typically, a majority of the tufts in such a tufted material will collapse and close off the openings 20. FIG. 2 schematically shows an example of a tuft 12 after it has collapsed. In FIG. 2, the tuft 12 has folded over to the left side. FIG. 3 is an image showing a nonwoven material with several upwardly-oriented tufts, all of which have folded over to the side. However, not all of the tufts 12 will collapse and fold over to the same side. Often, some tufts 12 will fold to one side, and some tufts will fold to the other side. As a result of the collapse of the tufts 12, the openings 20 at the base of the tufts can close up, become slit-like, and virtually disappear.

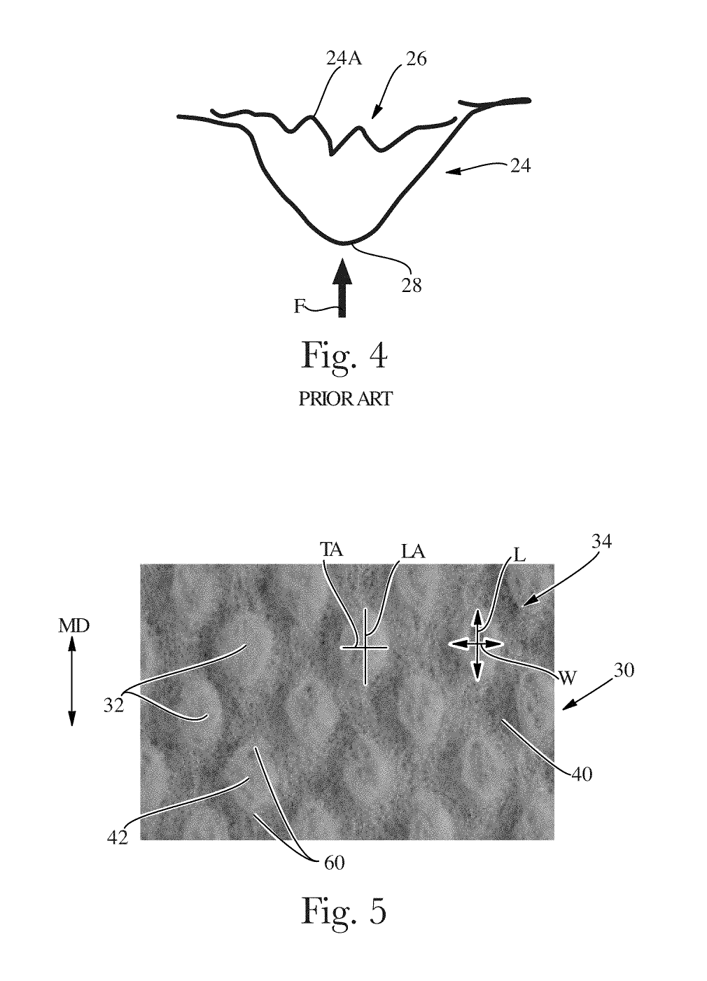

Prior art nonwoven materials with certain other types of three dimensional deformations, such as conical structures, can also be subject to collapse when compressed. As shown in FIG. 4, conical structures 24 will not necessarily fold over as will certain tufted structures when subjected to compressive forces F. However, conical structures 24 can be subject to collapse in that their relatively wide base opening 26 and smaller tip 28 causes the conical structure to push back toward the plane of the nonwoven material, such as to the configuration designated 24A.

The nonwoven materials of at least some forms of the present disclosure described herein are intended to better preserve the structure of discrete three-dimensional features in the nonwoven materials after compression.

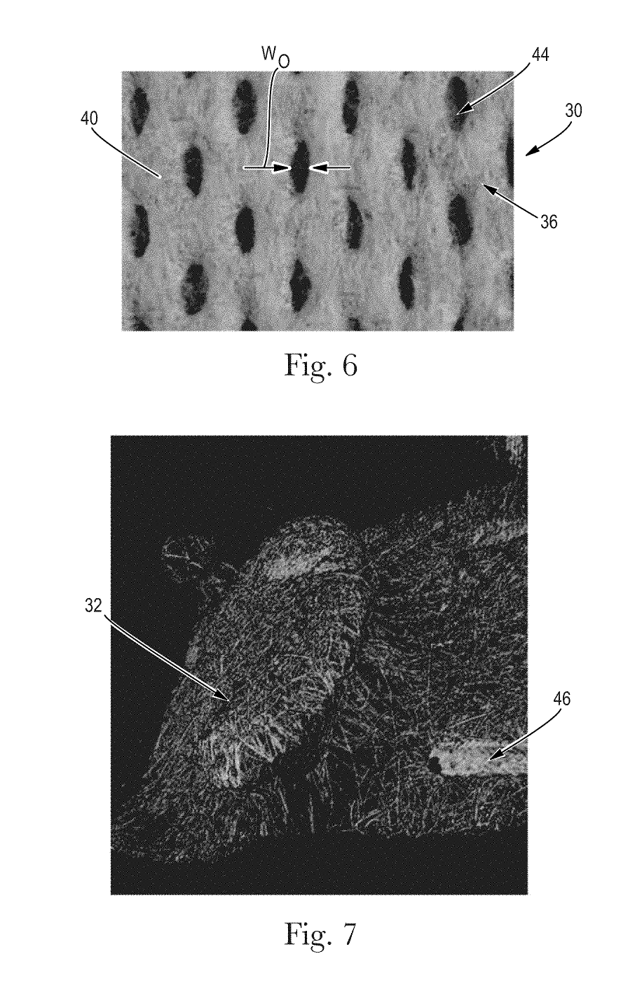

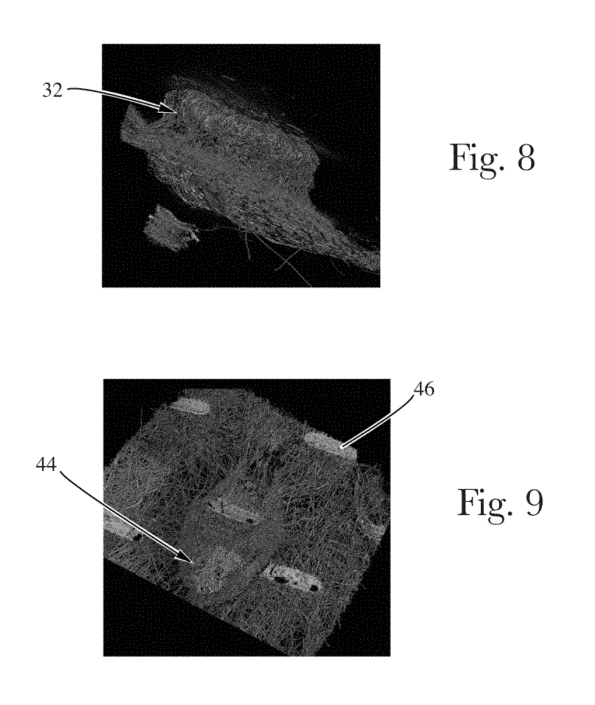

FIGS. 5-14 show examples of nonwoven materials 30 with three-dimensional deformations comprising protrusions 32 therein. The nonwoven materials 30 have a first surface 34, a second surface 36, and a thickness T therebetween (the thickness being shown in FIG. 12). FIG. 5 shows the first surface 34 of a nonwoven material 30 with the protrusions 32 that extend outward from the first surface 34 of the nonwoven material oriented upward. FIG. 6 shows the second surface 36 of a nonwoven material 30 such as that shown in FIG. 5, having three-dimensional deformations formed therein, with the protrusions oriented downward and the base openings 44 oriented upward. FIG. 7 is a Micro CT scan image showing a perspective view of a protrusion 32. FIG. 8 is a Micro CT scan image showing a side view of a protrusion 32 (of one of the longer sides of the protrusion). FIG. 9 is a Micro CT scan image showing a perspective view of a deformation with the opening 44 facing upward. The nonwoven materials 30 comprise a plurality of fibers 38 (shown in FIGS. 7-11 and 14). As shown in FIGS. 7 and 9, in some cases, the nonwoven material 30 may have a plurality of bonds 46 (such as thermal point bonds) therein to hold the fibers 38 together. Any such bonds 46 are typically present in the precursor material from which the nonwoven materials 30 are formed.

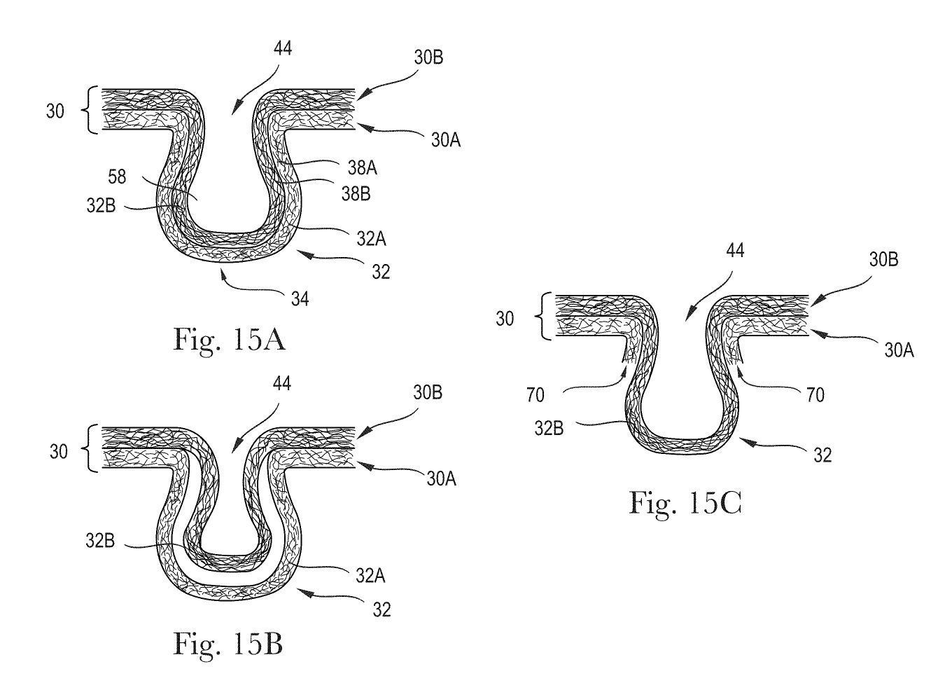

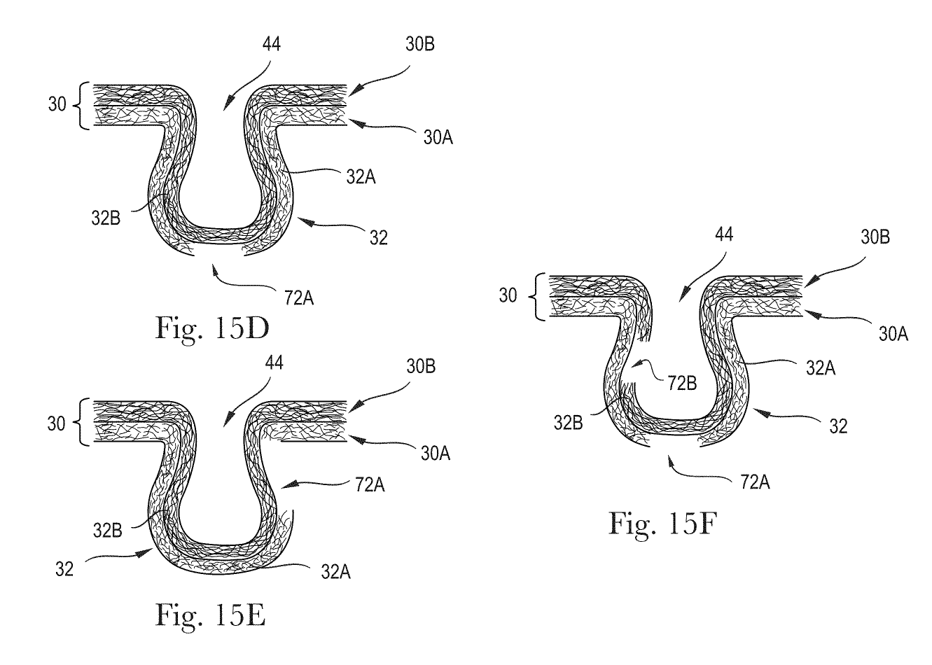

The protrusions 32 may, in some cases, be formed from looped fibers (which may be continuous) 38 that are pushed outward so that they extend out of the plane of the nonwoven web in the Z-direction. The protrusions 32 will typically comprise more than one looped fiber. In some cases, the protrusions 32 may be formed from looped fibers and at least some broken fibers. In addition, in the case of some types of nonwoven materials (such as carded materials, which are comprised of shorter fibers), the protrusions 32 may be formed from loops comprising multiple discontinuous fibers. Multiple discontinuous fibers in the form of a loop are shown as layer 30A in FIGS. 15A-15F. The looped fibers may be: aligned (that is, oriented in substantially the same direction); not be aligned; or, the fibers may be aligned in some locations within the protrusions 32, and not aligned in other parts of the protrusions.

In some cases, if male/female forming elements are used to form the protrusions 32, and the female forming elements substantially surround the male forming elements, the fibers in at least part of the protrusions 32 may remain substantially randomly oriented (rather than aligned), similar to their orientation in the precursor web(s). For example, in some cases, the fibers may remain substantially randomly oriented in the cap of the protrusions, but be more aligned in the side walls such that the fibers extend in the Z-direction from the base of the protrusions to the cap. In addition, if the precursor web comprises a multi-layer nonwoven material, the alignment of fibers can vary between layers, and can also vary between different portions of a given protrusion 32 within the same layer.

The nonwoven material 30 may comprise a generally planar first region 40 and the three-dimensional deformations may comprise a plurality of discrete integral second regions 42. The term “generally planar” is not meant to imply any particular flatness, smoothness, or dimensionality. Thus, the first region 40 can include other features that provide the first region 40 with a topography. Such other features can include, but are not limited to small projections, raised network regions around the base openings 44, and other types of features. Thus, the first region 40 is generally planar when considered relative to the second regions 42. The first region 40 can have any suitable plan view configuration. In some cases, the first region 40 is in the form of a continuous inter-connected network which comprises portions that surround each of the deformations.

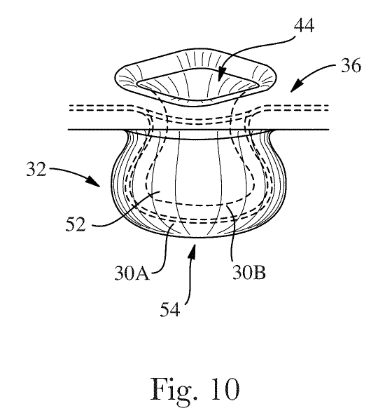

The term “deformation”, as used herein, includes both the protrusions 32 formed on one side of the nonwoven material and the base openings 44 formed in the opposing side of the material. The base openings 44 are most often not in the form of an aperture or a through-hole. The base openings 44 may instead appear as depressions. The base openings 44 can be analogized to the opening of a bag. A bag has an opening that typically does not pass completely through the bag. In the case of the present nonwoven materials 30, as shown in FIG. 10, the base openings 44 open into the interior of the protrusions 32.

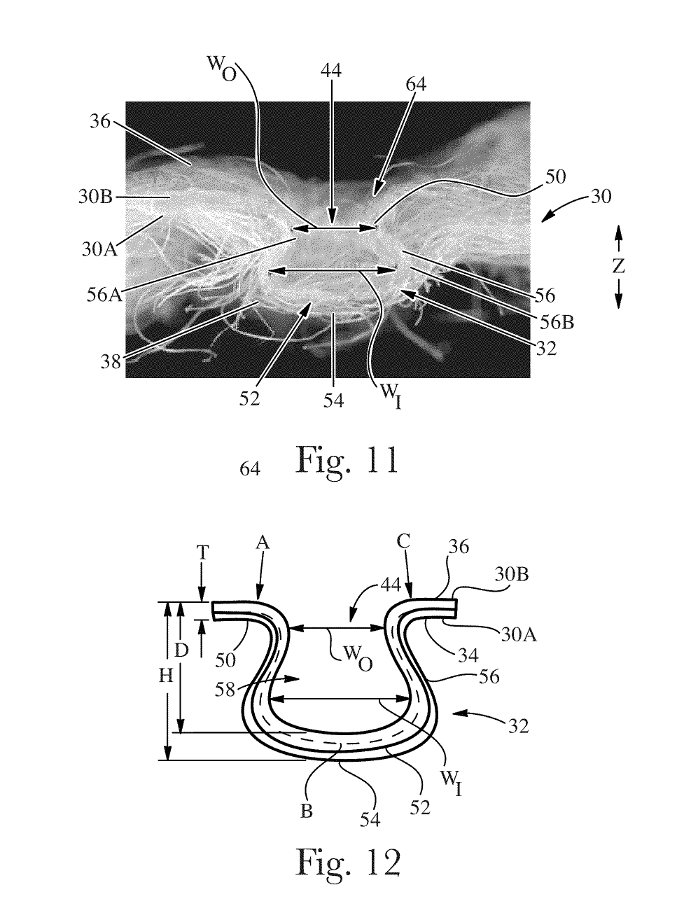

FIG. 11 shows one example of a multi-layer nonwoven material 30 having a three-dimensional deformation in the form of a protrusion 32 on one side of the material that provides a wide base opening 44 on the other side of the material. The dimensions of “wide” base openings are described in further detail below. In this case, the base opening 44 is oriented upward in the figure. When there is more than one nonwoven layer, the individual layers can be designated 30A, 30B, etc. The individual layers 30A and 30B each have first and second surfaces, which can be designated similarly to the first and second surfaces 34 and 36 of the nonwoven material (e.g., 34A and 36A for the first and second surfaces of the first layer 30A; and, 34B and 36B for the first and second surfaces of the second layer 30B).

As shown in FIGS. 11 and 12, the protrusions 32 comprise: a base 50 proximate the first surface 34 of the nonwoven material; an opposed enlarged distal portion or cap portion, or “cap” 52, that extends to a distal end 54; side walls (or “sides”) 56; an interior 58; and a pair of ends 60 (the latter being shown in FIG. 5). The “base” 50 of the protrusions 32 comprises the narrowest portion of the protrusion when viewed from one of the ends of the protrusion. The term “cap” does not imply any particular shape, other than it comprises the wider portion of the protrusion 32 that includes and is adjacent to the distal end 54 of the protrusion 32. The side walls 56 have an inside surface 56A and an outside surface 56B. As shown in FIGS. 11 and 12, the side walls 56 transition into, and may comprise part of the cap 52. Therefore, it is not necessary to precisely define where the side walls 56 end and the cap 52 begins. The cap 52 will have a maximum interior width, WI, between the inside surfaces 56A of the opposing side walls 56. The cap 52 will also have a maximum exterior width W between the outside surfaces 56B of the opposing side walls 56. The ends 60 of the protrusions 32 are the portions of the protrusions that are spaced furthest apart along the longitudinal axis, L, of the protrusions.

As shown in FIGS. 11 and 12, the narrowest portion of the protrusion 32 defines the base opening 44. The base opening 44 has a width Wo. The base opening 44 may be located (in the z-direction) between the plane defined by the second surface 36 of the material and the distal end 54 of the protrusion. As shown in FIGS. 11 and 12, the nonwoven material 30 may have an opening in the second surface 36 (the “second surface opening” 64) that transitions into the base opening 44 (and vice versa), and is the same size as, or larger than the base opening 44. The base opening 44 will, however, generally be discussed more frequently herein since its size will often be more visually apparent to the consumer in those forms where the nonwoven material 30 is placed in an article with the base openings 44 visible to the consumer. It should be understood that in certain forms, such as in some forms in which the base openings 44 face outward (for example, toward a consumer and away from the absorbent core in an absorbent article), it may be desirable for the base openings 44 not to be covered and/or closed off by another web.

As shown in FIG. 12, the protrusions 32 have a depth D measured from the second surface 36 of the nonwoven web to the interior of the protrusion at the distal end 54 of the protrusions. The protrusions 32 have a height H measured from the second surface 36 of the nonwoven web to the distal end 54 of the protrusions. In most cases the height H of the protrusions 32 will be greater than the thickness T of the first region 40. The relationship between the various portions of the deformations may be such that as shown in FIG. 11, when viewed from the end, the maximum interior width W_{I} of the cap 52 of the protrusions is wider than the width, Wo, of the base opening 44.

The protrusions 32 may be of any suitable shape. Since the protrusions 32 are three-dimensional, describing their shape depends on the angle from which they are viewed. When viewed from above (that is, perpendicular to the plane of the web, or plan view) such as in FIG. 5, suitable shapes include, but are not limited to: circular, diamond-shaped, rounded diamond-shaped, U.S. football-shaped, oval-shaped, clover-shaped, heart-shaped, triangle-shaped, tear-drop shaped, and elliptical-shaped. (The base openings 44 will typically have a shape similar to the plan view shape of the protrusions 32.) In other cases, the protrusions 32 (and base openings 44) may be non-circular. The protrusions 32 may have similar plan view dimensions in all directions, or the protrusions may be longer in one dimension than another. That is, the protrusions 32 may have different length and width dimensions. If the protrusions 32 have a different length than width, the longer dimension will be referred to as the length of the protrusions. The protrusions 32 may, thus, have a ratio of length to width, or an aspect ratio. The aspect ratios can range from about 1:1 to about 10:1.

As shown in FIG. 5, the protrusions 32 may have a width, W, that varies from one end 60 to the opposing end 60 when the protrusions are viewed in plan view. The width W may vary with the widest portion of the protrusions in the middle of the protrusions, and the width of the protrusions decreasing at the ends 60 of the protrusions. In other cases, the protrusions 32 could be wider at one or both ends 60 than in the middle of the protrusions. In still other cases, protrusions 32 can be formed that have substantially the same width from one end of the protrusion to the other end of the protrusion. If the width of the protrusions 32 varies along the length of the protrusions, the portion of the protrusion where the width is the greatest is used in determining the aspect ratio of the protrusions.

When the protrusions 32 have a length L that is greater than their width W, the length of the protrusions may be oriented in any suitable direction relative to the nonwoven material 30. For example, the length of the protrusions 32 (that is, the longitudinal axis, LA, of the protrusions) may be oriented in the machine direction, the cross-machine direction, or any desired orientation between the machine direction and the cross-machine direction. The protrusions 32 also have a transverse axis TA generally orthogonal to the longitudinal axis LA in the MD-CD plane. In the form shown in FIGS. 5 and 6, the longitudinal axis LA is parallel to the MD. In some forms, all the spaced apart protrusions 32 may have generally parallel longitudinal axes LA.

The protrusions 32 may have any suitable shape when viewed from the side. Suitable shapes include those in which there is a distal portion or “cap” with an enlarged dimension and a narrower portion at the base when viewed from at least one side. The term “cap” is analogous to the cap portion of a mushroom. (The cap does not need to resemble that of any particular type of mushroom. In addition, the protrusions 32 may, but need not, have a mushroom-like stem portion.) In some cases, the protrusions 32 may be referred to as having a bulbous shape when viewed from the end 60, such as in FIG. 11. The term “bulbous”, as used herein, is intended to refer to the configuration of the protrusions 32 as having a cap 52 with an enlarged dimension and a narrower portion at the base when viewed from at least one side (particularly when viewing from one of the shorter ends 60) of the protrusion 32. The term “bulbous” is not limited to protrusions that have a circular or round plan view configuration that is joined to a columnar portion. The bulbous shape, in the form shown (where the longitudinal axis LA of the deformations 32 is oriented in the machine direction), may be most apparent if a section is taken along the transverse axis TA of the deformation (that is, in the cross-machine direction). The bulbous shape may be less apparent if the deformation is viewed along the length (or longitudinal axis LA) of the deformation such as in FIG. 8.

The protrusions 32 may comprise fibers 38 that at least substantially surround the sides of the protrusions. This means that there are multiple fibers that extend (e.g., in the Z-direction) from the base 50 of the protrusions 32 to the distal end 54 of the protrusions, and contribute to form a portion of the sides 56 and cap 52 of a protrusion. In some cases, the fibers may be substantially aligned with each other in the Z-direction in the sides 56 of the protrusions 32. The phrase “substantially surround”, thus, does not require that each individual fiber be wrapped in the X-Y plane substantially or completely around the sides of the protrusions. If the fibers 38 are located completely around the sides of the protrusions, this would mean that the fibers are located 360° around the protrusions. The protrusions 32 may be free of large openings at their ends 60, such as those openings 18 at the leading end and trailing end of the tufts shown in FIG. 1. In some cases, the protrusions 32 may have an opening at only one of their ends, such as at their trailing end. The protrusions 32 also differ from embossed structures such as shown in FIG. 4. Embossed structures typically do not have distal portions that are spaced perpendicularly away (that is, in the Z-direction) from their base that are wider than portions that are adjacent to their base, as in the case of the cap 52 on the present protrusions 32.

The protrusions 32 may have certain additional characteristics. As shown in FIGS. 11 and 12, the protrusions 32 may be substantially hollow. As used herein, the term “substantially hollow” refers to structures which the protrusions 32 are substantially free of fibers in interior of protrusions. The term “substantially hollow”, does not, however, require that the interior of the protrusions must be completely free of fibers. Thus, there can be some fibers inside the protrusions. “Substantially hollow” protrusions are distinguishable from filled three-dimensional structures, such as those made by laying down fibers, such as by airlaying or carding fibers onto a forming structure with recesses therein.

The side walls 56 of the protrusions 32 can have any suitable configuration. The configuration of the side walls 56, when viewed from the end of the protrusion such as in FIG. 11, can be linear or curvilinear, or the side walls can be formed by a combination of linear and curvilinear portions. The curvilinear portions can be concave, convex, or combinations of both. For example, the side walls 56 in the form shown in FIG. 11 comprise portions that are curvilinear concave inwardly near the base of the protrusions and convex outwardly near the cap of the protrusions. The sidewalls 56 and the area around the base opening 44 of the protrusions may, under 20× magnification, have a visibly significantly lower concentration of fibers per given area (which may be evidence of a lower basis weight or lower opacity) than the portions of the nonwoven in the unformed first region 40. The protrusions 32 may also have thinned fibers in the sidewalls 56. The fiber thinning, if present, will be apparent in the form of necked regions in the fibers 38 as seen in scanning electron microscope (SEM) images taken at 200× magnification. Thus, the fibers may have a first cross-sectional area when they are in the undeformed nonwoven precursor web, and a second cross-sectional area in the side walls 56 of the protrusions 32 of the deformed nonwoven web, wherein the first cross-sectional area is greater than the second cross-sectional area. The side walls 56 may also comprise some broken fibers as well. In some forms, the side walls 56 may comprise greater than or equal to about 30%, alternatively greater than or equal to about 50% broken fibers.

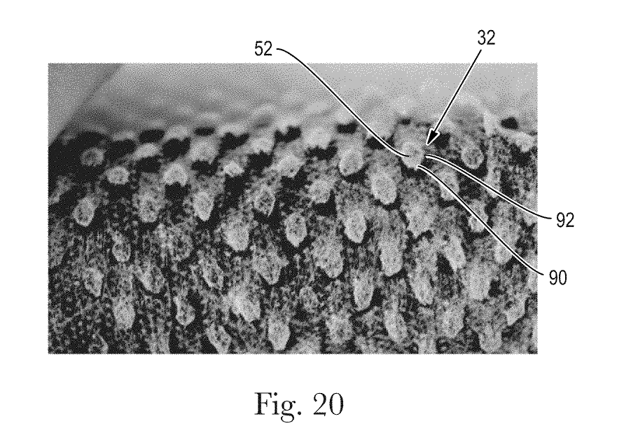

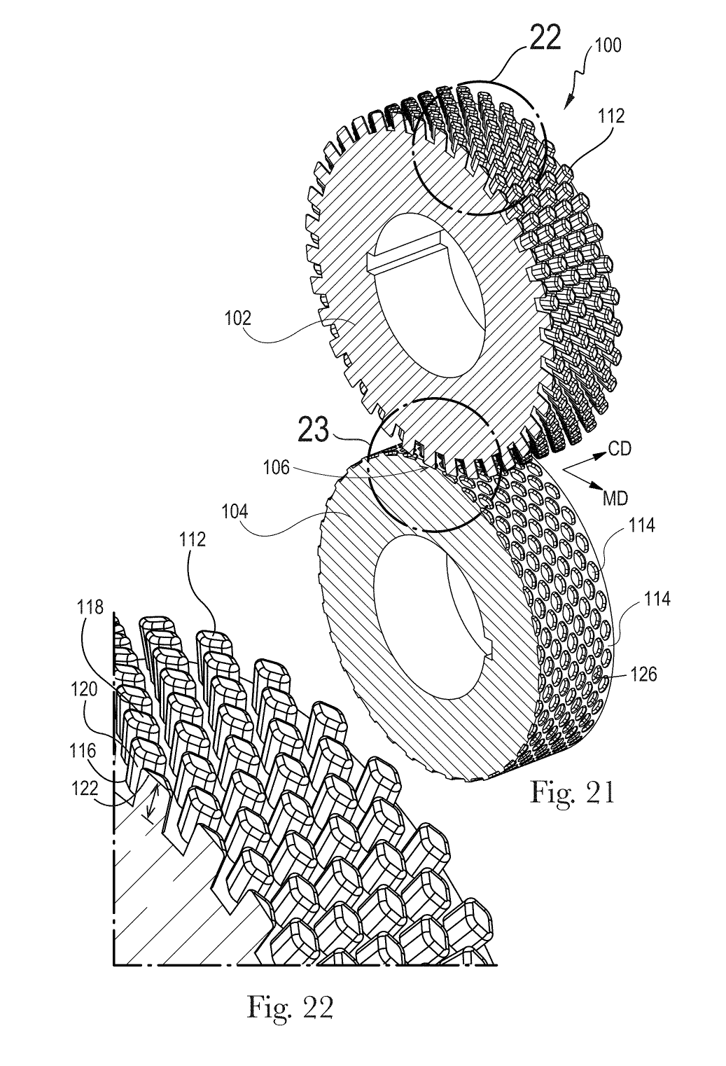

In some forms, the distal end 54 of the protrusions 32 may be comprised of original basis weight, non-thinned, and non-broken fibers. If the base opening 44 faces upward, the distal end 54 will be at the bottom of the depression that is formed by the protrusion. The distal end 54 will be free from apertures formed completely through the distal end. Thus, the nonwoven materials may be nonapertured. The term “apertures”, as used herein, refers to holes formed in the nonwovens after the formation of the nonwovens, and does not include the pores typically present in nonwovens. The term “apertures” also does not refer to irregular breaks (or interruptions) in the nonwoven material(s) such as shown in FIGS. 15D-15F and FIG. 20 resulting from localized tearing of the material(s) during the process of forming deformations therein, which breaks may be due to variability in the precursor material(s). The distal end 54 may have relatively greater fiber concentration in comparison to the remaining portions of the structure that forms the protrusions. The fiber concentration can be measured by viewing the sample under a microscope and counting the number of fibers within an area. As described in greater detail below, however, if the nonwoven web is comprised of more than one layer, the concentration of fibers in the different portions of the protrusions may vary between the different layers.

The protrusions 32 may be of any suitable size. The size of the protrusions 32 can be described in terms of protrusion length, width, caliper, height, depth, cap size, and opening size. (Unless otherwise stated, the length L and width W of the protrusions are the exterior length and width of the cap 52 of the protrusions.) The dimensions of the protrusions and openings can be measured before and after compression (under either a pressure of 7 kPa or 35 KPa, whichever is specified) in accordance with the Accelerated Compression Method described in the Test Methods section. The protrusions have a caliper that is measured between the same points as the height H, but under a 2 KPa load, in accordance with the Accelerated Compression Method. All dimensions of the protrusions and openings other than caliper (that is, length, width, height, depth, cap size, and opening size) are measured without pressure applied at the time of making the measurement using a microscope at 20× magnification.

In some forms, the length of the cap 52 may be in a range from about 1.5 mm to about 10 mm. In some forms, the width of the cap (measured where the width is the greatest) may be in a range from about 1.5 mm to about 5 mm. The cap portion of the protrusions may have a plan view surface area of at least about 3 mm². In some forms, the protrusions may have a pre-compression height H that is in a range from about 1 mm to about 10 mm, alternatively from about 1 mm to about 6 mm. In some forms, the protrusions may have a post-compression height H that is in a range from about 0.5 mm to about 6 mm, alternatively from about 0.5 mm to about 1.5 mm. In some forms, the protrusions may have a depth D, in an uncompressed state that is in a range from about 0.5 mm to about 9 mm, alternatively from about 0.5 mm to about 5 mm. In some forms, the protrusions may have a depth D, after compression that is in a range from about 0.25 mm to about 5 mm, alternatively from about 0.25 mm to about 1 mm.

The nonwoven material 30 can comprise a composite of two or more nonwoven materials that are joined together. In such a case, the fibers and properties of the first layer will be designated accordingly (e.g., the first layer is comprised of a first plurality of fibers), and the fibers and properties of the second and subsequent layers will be designated accordingly (e.g., the second layer is comprised of a second plurality of fibers). In a two or more layer structure, there are a number of possible configurations the layers may take following the formation of the deformations therein. These will often depend on the extensibility of the nonwoven materials used for the layers. It is desirable that at least one of the layers have deformations which form protrusions 32 as described herein in which, along at least one cross-section, the width of the cap 52 of the protrusions is greater than the width of the base opening 44 of the deformations. For example, in a two layer structure where one of the layers will serve as the topsheet of an absorbent article and the other layer will serve as an underlying layer (such as an acquisition layer), the layer that has protrusions therein may comprise the topsheet layer. The layer that most typically has a bulbous shape will be the one which is in contact with the male forming member during the process of deforming the web. FIG. 15A-FIG. 15E show different alternative forms of three-dimensional protrusions 32 in multiple layer materials.

In certain forms, such as shown in FIGS. 11, 12, and 15A, similar-shaped looped fibers may be formed in each layer of multiple layer nonwoven materials, including in the layer 30A that is spaced furthest from the discrete male forming elements during the process of forming the protrusions 32 therein, and in the layer 30B that is closest to the male forming elements during the process. In the protrusions 32, portions of one layer such as 30B may fit within the other layer, such as 30A. These layers may be referred to as forming a “nested” structure in the protrusions 32. Formation of a nested structure may require the use of two (or more) highly extensible nonwoven precursor webs. In the case of two layer materials, nested structures may form two complete loops, or (as shown in some of the following drawing figures) two incomplete loops of fibers.

As shown in FIG. 15A, a three-dimensional protrusion 32 comprises protrusions 32A formed in the first layer 30A and protrusions 32B formed in the second layer 30B. In one form, the first layer 30A may be incorporated into an absorbent article as an acquisition layer, and the second layer 30B may be a topsheet, and the protrusions formed by the two layers may fit together (that is, are nested). In this form, the protrusions 32A and 32B formed by the first and second layers 30A and 30B fit closely together. The three-dimensional protrusion 32A comprises a plurality of fibers 38A and the three-dimensional protrusion 32B comprises a plurality of fibers 38B. The three-dimensional protrusion 32B is nested into the three-dimensional protrusion 32A. In the form shown, the fibers 38A in the first layer 30A are shorter in length than the fibers 38B in the second layer 30B. In other forms, the relative length of fibers in the layers may be the same, or in the opposite relationship wherein the fibers in the first layer are longer than those in the second layer. In addition, in this form, and any of the other forms described herein, the nonwoven layers can be inverted when incorporated into an absorbent article, or other article, so that the protrusions 32 face upward (or outward). In such a case, the material suitable for the topsheet will be used in layer 30A, and material suitable for the underlying layer will be used in layer 30B.

FIG. 15B shows that the nonwoven layers need not be in a contacting relationship within the entirety of the protrusion 32. Thus, the protrusions 32A and 32B formed by the first and second layers 30A and 30B may have different heights and/or widths. The two materials may have substantially the same shape in the protrusion 32 as shown in FIG. 15B (where one of the materials has the same the curvature as the other). In other forms, however, the layers may have different shapes. It should be understood that FIG. 15B shows only one possible arrangement of layers, and that many other variations are possible, but that as in the case of all the figures, it is not possible to provide a drawing of every possible variation.

As shown in FIG. 15C, one of the layers, such as first layer 30A (e.g., an acquisition layer) may be ruptured in the area of the three-dimensional protrusion 32. As shown in FIG. 15C, the protrusions 32 are only formed in the second layer 30B (e.g., the topsheet) and extend through openings in the first layer 30A. That is, the three-dimensional protrusion 32B in the second layer 30B interpenetrates the ruptured first layer 30A. Such a structure may place the topsheet in direct contact an underlying distribution layer or absorbent core, which may lead to improved dryness. In such an form, the layers are not considered to be “nested” in the area of the protrusion. (In the other forms shown in FIGS. 15D-15F, the layers would still be considered to be “nested”.) Such a structure may be formed if the material of the second layer 30B is much more extensible than the material of the first layer 30A. In such a case, the openings can be formed by locally rupturing first precursor web by the process described in detail below. The ruptured layer may have any suitable configuration in the area of the protrusion 32. Rupture may involve a simple splitting open of first precursor web, such that the opening in the first layer 30A remains a simple two-dimensional aperture. However, for some materials, portions of the first layer 30A can be deflected or urged out-of-plane (i.e., out of the plane of the first layer 30A) to form flaps 70. The form and structure of any flaps is highly dependent upon the material properties of the first layer 30A. Flaps can have the general structure shown in FIG. 15C. In other forms, the flaps 70 can have a more volcano-like structure, as if the protrusion 32B is erupting from the flaps.

Alternatively, as shown in FIGS. 15D-15F, one or both of the first layer 30A and the second layer 30B may be interrupted (or have a break therein) in the area of the three-dimensional protrusion 32. FIGS. 15D and 15E show that the three-dimensional protrusion 32A of the first layer 30A may have an interruption 72A therein. The three-dimensional protrusion 32B of the non-interrupted second layer 30B may coincide with and fit together with the three-dimensional protrusion 32A of the interrupted first layer 30A. Alternatively, FIG. 15F shows an form in which both the first and second layers 30A and 30B have interruptions, or breaks, therein (72A and 72B, respectively). In this case, the interruptions in the layers 30A and 30B are in different locations in the protrusion 32. FIGS. 15D-15F show non-predetermined random or inconsistent breaks in the materials typically formed by random fiber breakage, which are generally misaligned and can be in the first or second layer, but are not typically aligned and completely through both layers. Thus, there typically will not be an aperture formed completely through all of the layers at the distal end 54 of the protrusions 32.

For dual layer and other multiple layer structures, the basis weight distribution (or the concentration of fibers) within the deformed material 30, as well as the distribution of any thermal point bonds 46 can be different between the layers. As used herein, the term “fiber concentration” has a similar meaning as basis weight, but fiber concentration refers to the number of fibers/given area, rather than g/area as in basis weight. In the case of bond sites 46, the fibers may be melted which may increase the density of the material in the bond sites 46, but the number of fibers will typically be the same as before melting.

Some such dual and multiple layer nonwoven materials may be described in terms of such differences between layers, without requiring one or more of the other features described herein (such as characteristics of the cap portion; controlled collapse under compression; and varying width of the protrusions). Of course such dual and multiple layer nonwoven materials may have any of these other features.

In such dual and multiple layer nonwoven materials each of the layers comprises a plurality of fibers, and in certain forms, the protrusions 32 will be formed from fibers in each of the layers. For example, one of the layers, a first layer, may form the first surface 34 of the nonwoven material 30, and one of the layers, a second layer, may form the second surface 36 of the nonwoven material 30. A portion of the fibers in the first layer form part of: the first region 40, the side walls 56 of the protrusions, and the distal ends 54 of the protrusions 32. A portion of the fibers in the second layer form part of: the first region 40, the side walls 56 of the protrusions, and the distal ends 54 of the protrusions 32.

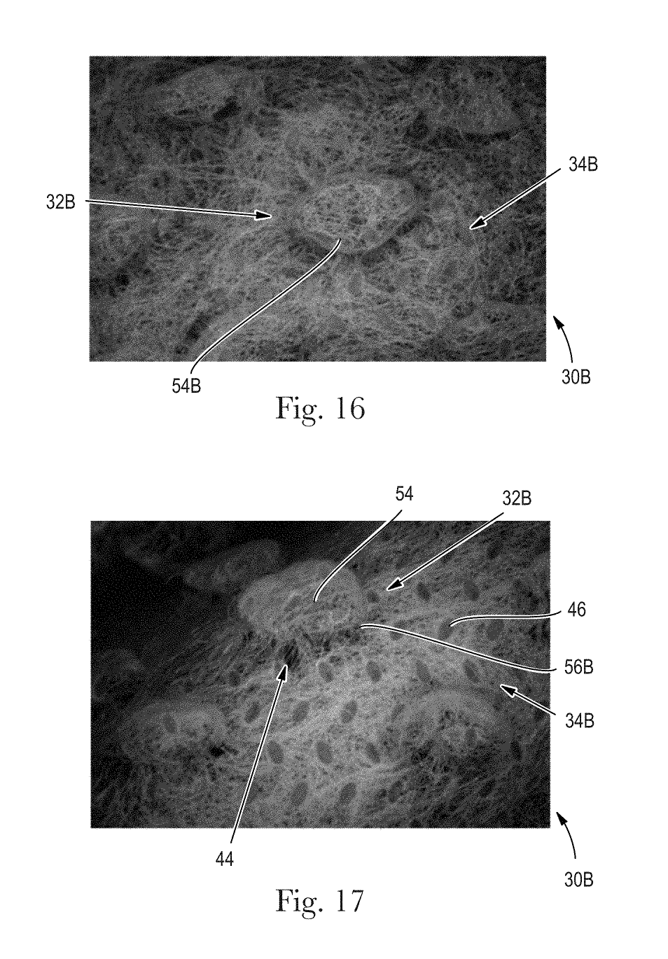

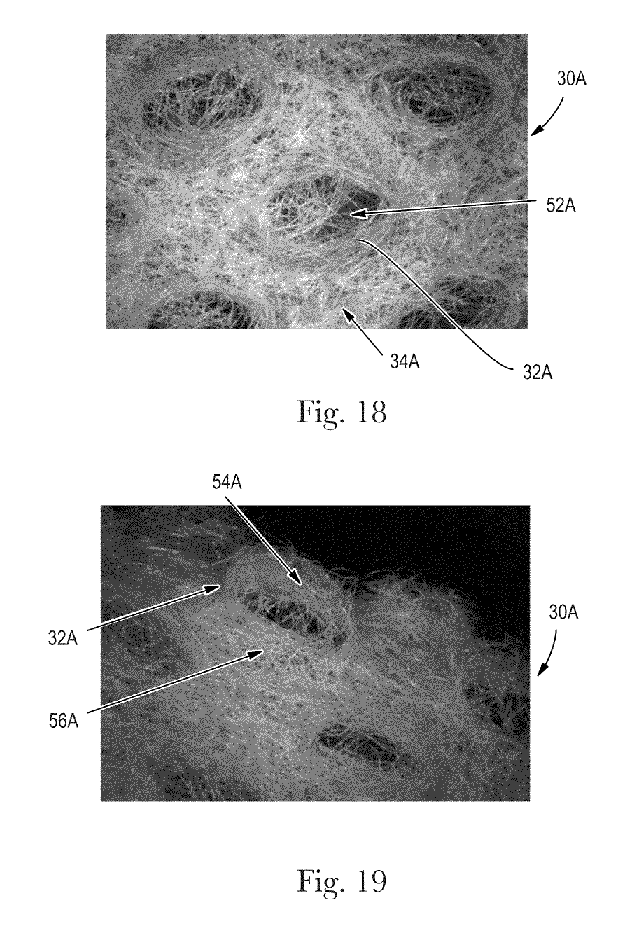

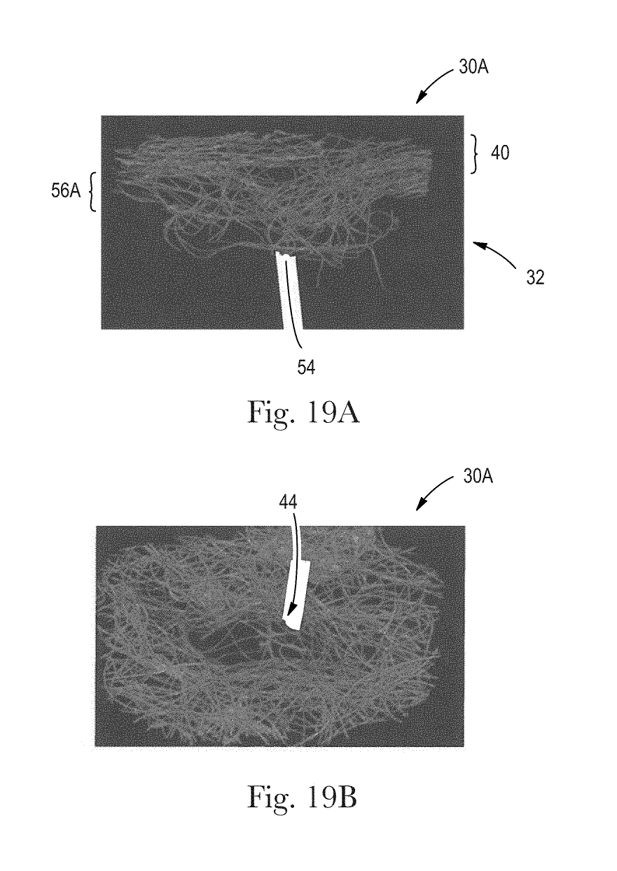

As shown in FIG. 16, the nonwoven layer in contact with the male forming element (e.g., 30B) may have a large portion at the distal end 54B of the protrusion 32B with a similar basis weight to the original nonwoven (that is, to the first region 40). As shown in FIG. 17, the basis weight in the sidewalls 56B of the protrusion 32B and near the base opening 44 may be lower than the basis weight of the first region 40 of the nonwoven layer and the distal end 54 of the protrusion 32B. As shown in FIG. 18, the nonwoven layer in contact with the female forming element (e.g., 30A) may, however, have significantly less basis weight in the cap 52A of the protrusion 32A than in the first region 40 of the nonwoven layer. As shown in FIG. 19, the sidewalls 56A of the protrusion 32A may have less basis weight than the first region 40 of the nonwoven. FIGS. 19A and 19B show that the nonwoven layer 30A in contact with the female forming element may have a fiber concentration that is greatest in the first region 40 (at the upper part of the image in FIG. 19A) and lowest at the distal end 54 of the protrusion 32. The fiber concentration in the side wall 56A, in this case, may be less than that of the first region 40, but greater than that at the distal end 54 of the protrusion 32.

Forming deformations in the nonwoven material may also affect the bonds 46 (thermal point bonds) within the layer (or layers). In some forms, the bonds 46 within the distal end 54 of the protrusions 32 may remain intact (not be disrupted) by the deformation process that formed the protrusions 32. In the side walls 56 of the protrusions 32, however, the bonds 46 originally present in the precursor web may be disrupted. When it is said that the bonds 46 may be disrupted, this can take several forms. The bonds 46 can be broken and leave remnants of a bond. In other cases, such as where the nonwoven precursor material is underbonded, the fibers can disentangle from a lightly formed bond site (similar to untying a bow), and the bond site will essentially disappear. In some cases, after the deformation process, the side walls 56 of at least some of the protrusions 32 may be substantially free (or completely free) of thermal point bonds.

Numerous forms of dual layer and other multiple layer structures are possible. For example, a nonwoven layer 30B such as that shown in FIGS. 16 and 17 could be oriented with its base openings facing upward, and could serve as a topsheet of a dual or multiple layer nonwoven structure (with at least one other layer serving as an acquisition layer). In this form, the bonds 46 within first region 40 of nonwoven layer 30B and the distal end 54 of the protrusions 32 remain intact. In the side walls 56 of the protrusions 32, however, the bonds 46 originally present in the precursor web are disrupted such that the side walls 56 are substantially free of thermal point bonds. Such a topsheet could be combined with an acquisition layer in which the concentration of fibers within the layer 30A in the first region 40 and the distal end 54 of the protrusions 32 is also greater than the concentration of fibers in the side walls 56 of the protrusions 32.

In other forms, the acquisition layer 30A described in the preceding paragraph may have thermal point bonds 46 within first region 40 of nonwoven layer 30B and the distal end 54 of the protrusions 32 that remain intact. In the side walls 56 of the protrusions 32, however, the bonds 46 originally present in the precursor web comprising the acquisition layer 30A are disrupted such that the side walls 56 of the acquisition layer 30A are substantially free of thermal point bonds. In other cases, the thermal point bonds in the acquisition layer 30A at the top of the protrusions 32 may also be disrupted so that the distal end 54 of at least some of the protrusions are substantially or completely free of thermal point bonds.

In other forms, a dual layer or multiple layer structure may comprise a topsheet and an acquisition layer that is oriented with its base openings facing upward in which the concentration of fibers at the distal end 54 of each layer (relative to other portions of the layer) differs between layers. For example, in one form, in the layer that forms the topsheet (second layer), the concentration of fibers in the first region and the distal ends of the protrusions are each greater than the concentration of fibers in the side walls of the protrusions. In the layer that forms the acquisition layer (first layer), the concentration of fibers in the first region of the acquisition layer may be greater than the concentration of fibers in the distal ends of the protrusions. In a variation of this form, the concentration of fibers in the first region of the first layer (acquisition layer) is greater than the concentration of fibers in the side walls of the protrusions in the first layer, and the concentration of fibers in the side walls of the protrusions in the first layer is greater than the concentration of fibers forming the distal ends of the protrusions in the first layer. In some forms in which the first layer comprises a spunbond nonwoven material (in which the precursor material had thermal point bonds distributed substantially evenly throughout), a portion of the fibers that form the first region in the first layer comprise thermal point bonds, and the portion of the fibers in the first layer forming the side walls and distal ends of at least some of the protrusions may be substantially free of thermal point bonds. In these forms, in at least some of the protrusions, at least some of the fibers in the first layer may form a nest or circle around (that is, encircle) the perimeter of the protrusion at the transition between the wide wall and the base of the protrusion as shown in FIG. 19.

The base openings 44 can be of any suitable shape and size. The shape of the base opening 44 will typically be similar to, or the same as, the plan view shape of the corresponding protrusions 32. The base opening 44 may have a width that is greater than about any of the following dimensions before (and after compression): 0.5 mm, 0.7 mm, 0.8 mm, 0.9 mm, 1 mm, or any 0.1 mm increment above 1 mm. The width of the base opening 44 may be in a range that is from any of the foregoing amounts up to about 4 mm, or more. The base openings 44 may have a length that ranges from about 1.5 mm or less to about 10 mm, or more. The base openings 44 may have an aspect ratio that ranges from about 1:1 to 20:1, alternatively from about 1:1 to 10:1. Measurements of the dimensions of the base opening can be made on a photomicrograph. When the size of the width of the base opening 44 is specified herein, it will be appreciated that if the openings are not of uniform width in a particular direction, the width, Wo, is measured at the widest portion as shown in FIG. 6. The nonwoven materials of the present disclosure and the method of making the same may create deformations with a wider opening than certain prior structures which have a narrow base. This allows the base openings 44 to be more visible to the naked eye. The width of the base opening 44 is of interest because, being the narrowest portion of the opening, it will be most restrictive of the size of the opening. The deformations retain their wide base openings 44 after compression perpendicular to the plane of the first region 40.

The deformations may compress under load. In some cases, it may be desirable that the load is low enough so that, if the nonwoven is worn against a wearer's body, with the deformations in contact with the wearer's body, the deformations will be soft and will not imprint the skin. This applies in cases where either the protrusions 32 or the base openings 44 are oriented so that they are in contact with the wearer's body. For example, it may be desirable for the deformations to compress under pressures of 2 kPa or less. In other cases, it will not matter if the deformations imprint the wearer's skin. It may be desirable for at least one of the protrusions 32 in the nonwoven material 30 to collapse or buckle in the controlled manner described below under the 7 kPa load when tested in accordance with the Accelerated Compression Method in the Test Methods section below. Alternatively, at least some, or in other cases, a majority of the protrusions 32 may collapse in the controlled manner described herein. Alternatively, substantially all of the protrusions 32 may collapse in the controlled manner described herein. The ability of the protrusions 32 to collapse may also be measured under a load of 35 kPa. The 7 kPa and 35 kPa loads simulate manufacturing and compression packaging conditions. Wear conditions can range from no or limited pressure (if the wearer is not sitting on the absorbent article) up to 2 kPa, 7 kPa, or more.

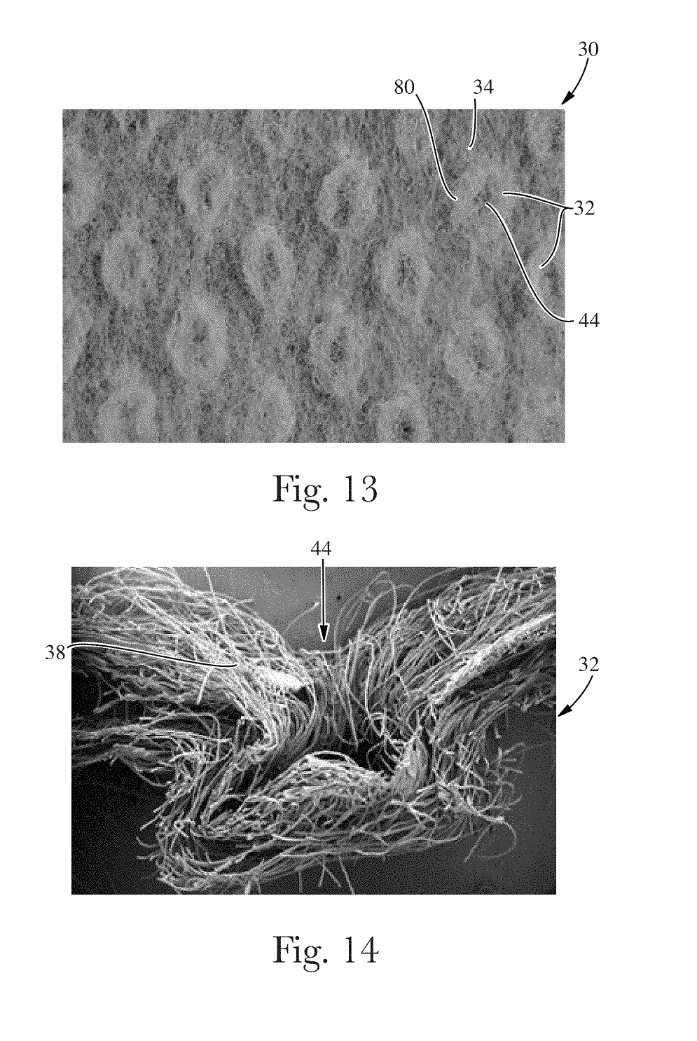

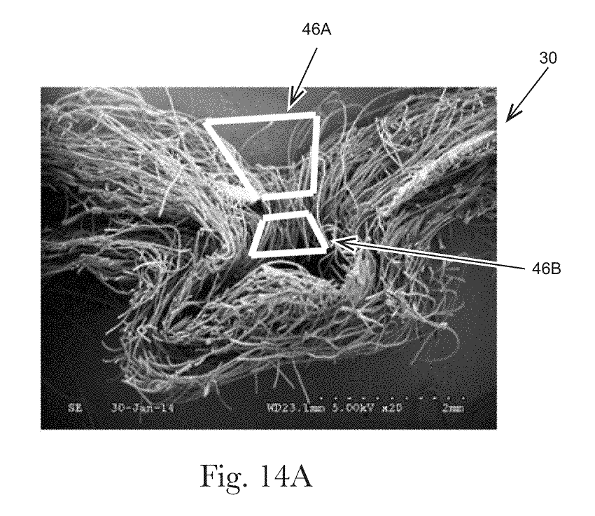

The protrusions 32 may collapse in a controlled manner after compression to maintain the wide opening 44 at the base. FIG. 13 shows the first surface 34 of a nonwoven material 30 according to the present disclosure after it has been subjected to compression. FIG. 14 is a side view of a single downwardly-oriented protrusion 32 after it has been subjected to compression. As shown in FIG. 13, when the protrusions 32 have been compressed, there appears to be a higher concentration of fibers in the form of a ring of increased opacity 80 around the base opening 44. When a compressive force is applied to the nonwoven materials, the side walls 56 of the protrusions 32 may collapse in a more desirable/controlled manner such that the side walls 56 become concave and fold into regions of overlapping layers (such as into an s-shape/accordion-shape). The ring of increased opacity 80 represents folded layers of material. In other words, the protrusions 32 may have a degree of dimensional stability in the X-Y plane when a Z-direction force is applied to the protrusions. It is not necessary that the collapsed configuration of the protrusions 32 be symmetrical, only that the collapsed configuration prevent the protrusions 32 from flopping over or pushing back into the original plane of the nonwoven, and significantly reducing the size of the base opening (for example, by 50% or more). For example, as shown in FIG. 14, the left side of the protrusion 32 can form a z-folded structure, and the right side of the protrusion does not, but still appears, when viewed from above, to have higher opacity due to a degree of overlapping of the material in the folded portion. Without wishing to be bound to any particular theory, it is believed that the wide base opening 44 and large cap 52 (greater than the width of the base opening 44), combined with the lack of a pivot point, causes the protrusions 32 to collapse in a controlled manner (prevents the protrusion 32 from flopping over). Thus, the protrusions 32 are free of a hinge structure that would otherwise permit them to fold to the side when compressed. The large cap 52 also prevents the protrusion 32 from pushing back into the original plane of the nonwoven.

Referring to FIGS. 11, 12, and 14A, the opening in the second surface 36 (the “second surface opening” 64) that transitions into the base opening 44 (and vice versa), may be larger than the base opening 44 after compression of the textured nonwoven material 30, as described herein. Generally, a transition from the surface opening 64 to the base opening 44 can take on a conical or frustoconical shape, i.e., frustum 46A, where the base of the frustum 46A is oriented toward the second surface opening 64. Past the base opening 44, the interior of the protrusion 32 may form a second frustum 46B, which has a base oriented toward the distal end 54 of the protrusion 32.

The deformations can be disposed in any suitable density across the surface of the nonwoven material 30. The deformations may, for example, be present in a density of: from about 5 to about 100 deformations; alternatively from about 10 to about 50 deformations; alternatively from about 20 to about 40 deformations, in an area of 10 cm².

The deformations can be disposed in any suitable arrangement across the plane of the nonwoven material. Suitable arrangements include, but are not limited to: staggered arrangements, and zones.

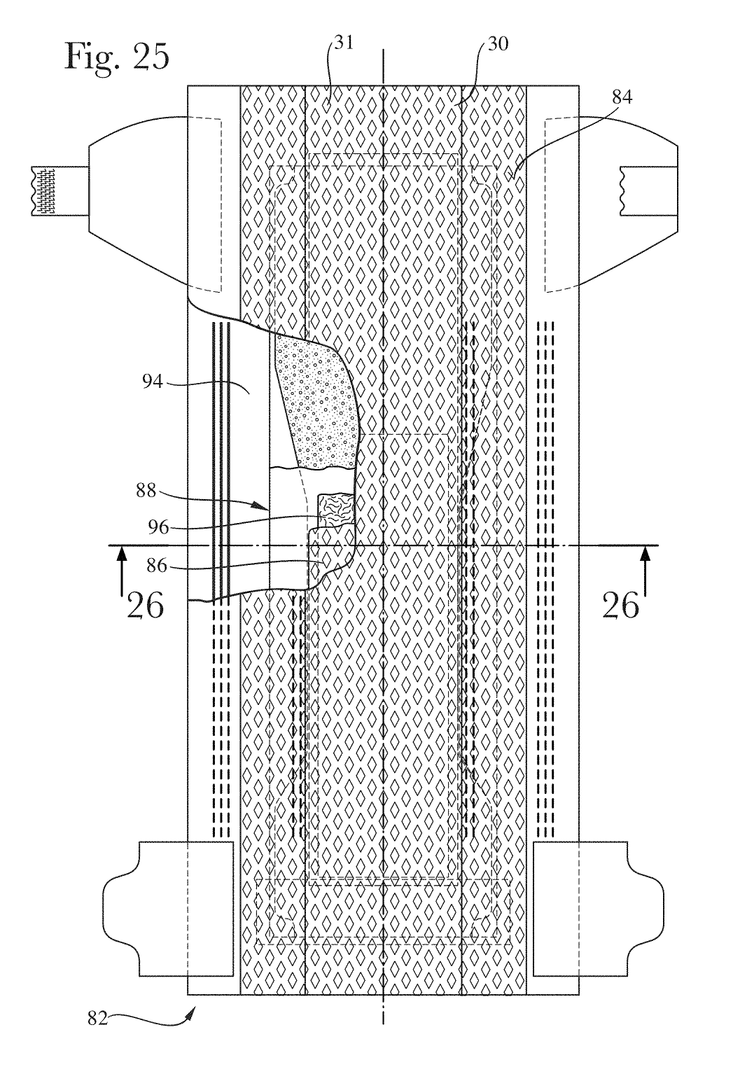

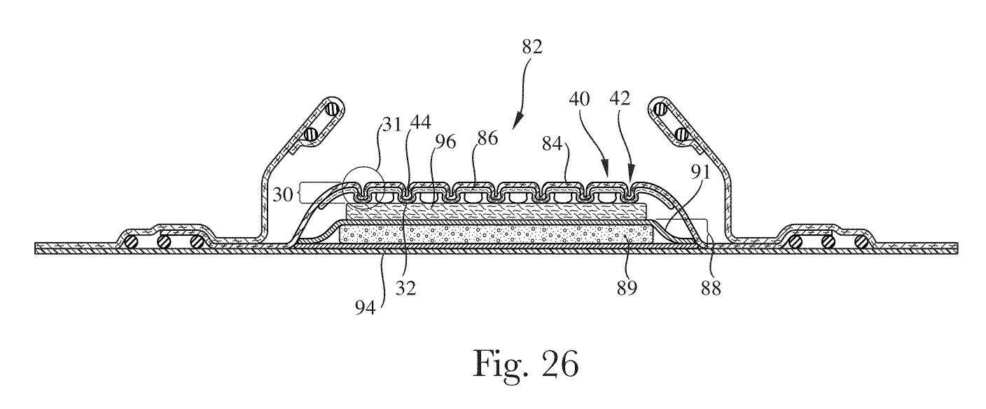

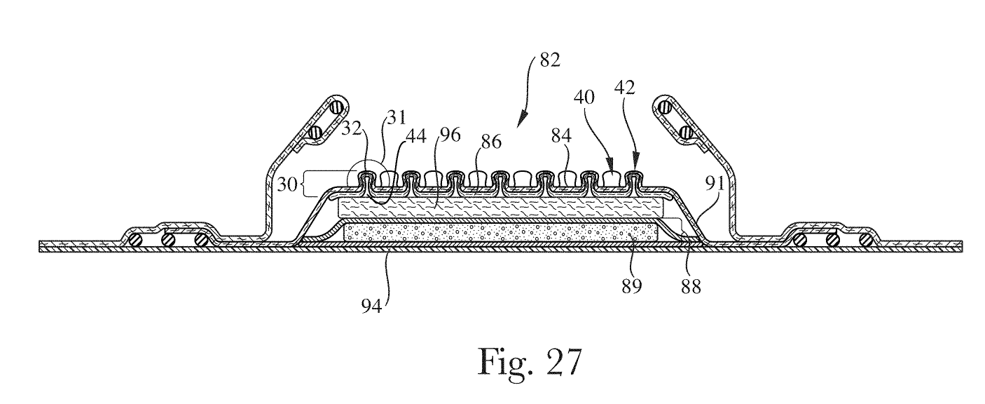

The nonwoven webs 30 described herein can comprise any suitable component or components of an absorbent article. For example, the nonwoven webs can comprise the topsheet of an absorbent article, or as shown in FIG. 25, if the nonwoven web 30 comprises more than one layer, the nonwoven web can comprise a combined topsheet 84 and acquisition layer 86 of an absorbent article, such as diaper 82. The diaper 82 shown in FIGS. 25-27 also comprises an absorbent core 88, a backsheet 94, and a distribution layer 96. The nonwoven materials of the present disclosure may also form an outer cover of an absorbent article, such as backsheet 94. The nonwoven webs 30 can be placed in an absorbent article with the deformations 31 in any suitable orientation. For example, the protrusions 32 can be oriented up or down. In other words, the protrusions 32 may be oriented toward the absorbent core 88 as shown in FIG. 26. Thus, for example, it may be desirable for the protrusions 32 to point inward toward the absorbent core 88 in a diaper (that is, away from the body-facing side and toward the garment-facing side), or other absorbent article. Alternatively, the protrusions 32 may be oriented so that they extend away from the absorbent core of the absorbent article as shown in FIG. 27. In still other forms, the nonwoven webs 30 can be made so that they have some protrusions 32 that are oriented upward, and some that are oriented downward. Without wishing to be bound to any particular theory, it is believed that such a structure may be useful in that the protrusions that are oriented upward can be more effective for cleaning the body from exudates, while the protrusions that are oriented downward can be more effective for absorption of exudates into the absorbent core. Therefore, without being bound to theory, a combination of these two protrusion orientations will offer advantage that the same product can fulfill the two functions.

A two or more layer nonwoven structure may provide fluid handling benefits. If the layers are integrated together, and the protrusions 32 are oriented toward the absorbent core, they may also provide a dryness benefit. It may be desirable, on the other hand, for the protrusions 32 to point outward, away from the absorbent core in a pad for a wet or dry mop to provide a cleaning benefit. In some forms, when the nonwoven web 30 is incorporated into an absorbent article, the underlying layers can be either substantially, or completely free, of tow fibers. Suitable underlying layers that are free of tow fibers may, for example, comprise a layer or patch of cross-linked cellulose fibers. In some cases, it may be desirable that the nonwoven material 30 is not entangled with (that is, is free from entanglement with) another web.

The layers of the nonwoven structure (e.g., a topsheet and/or acquisition layer) may be colored. Color may be imparted to the webs in any suitable manner including, but not limited to by color pigmentation. The term “color pigmentation” encompasses any pigments suitable for imparting a non-white color to a web. This term therefore does not include “white” pigments such as TiO₂ which are typically added to the layers of conventional absorbent articles to impart them with a white appearance. Pigments are usually dispersed in vehicles or substrates for application, as for instance in inks, paints, plastics or other polymeric materials. The pigments may for example be introduced in a polypropylene masterbatch. A masterbatch comprises a high concentration of pigment and/or additives which are dispersed in a carrier medium which can then be used to pigment or modify the virgin polymer material into a pigmented bicomponent nonwoven. An example of suitable colored masterbatch material that can be introduced is Pantone color 270 Sanylen violet PP 42000634 ex Clariant, which is a PP resin with a high concentration of violet pigment. Typically, the amount of pigments introduced by weight of the webs may be of from 0.3%-2.5%. Alternatively, color may be imparted to the webs by way of impregnation of a colorant into the substrate. Colorants such as dyes, pigments, or combinations may be impregnated in the formation of substrates such as polymers, resins, or nonwovens. For example, the colorant may be added to molten batch of polymer during fiber or filament formation.

### Precursor Materials

The nonwoven materials of the present disclosure can be made of any suitable nonwoven materials (“precursor materials”). The nonwoven webs can be made from a single layer, or multiple layers (e.g., two or more layers). If multiple layers are used, they can be comprised of the same type of nonwoven material, or different types of nonwoven materials. In some cases, the precursor materials may be free of any film layers.

The fibers of the nonwoven precursor material(s) can be made of any suitable materials including, but not limited to natural materials, synthetic materials, and combinations thereof. Suitable natural materials include, but are not limited to cellulose, cotton, cotton linters, bagasse, wool fibers, silk fibers, etc. Cotton materials may be hydrophobic or hydrophilic. Cellulose fibers can be provided in any suitable form, including but not limited to individual fibers, fluff pulp, drylap, liner board, etc. Suitable synthetic materials include, but are not limited to nylon, rayon and polymeric materials. Suitable polymeric materials include, but are not limited to: polyethylene (PE), polyester, polyethylene terephthalate (PET), polypropylene (PP), and co-polyester. In some forms, however, the nonwoven precursor materials can be either substantially, or completely free, of one or more of these materials. For example, in some forms, the precursor materials may be substantially free of cellulose, and/or exclude paper materials. In some forms, one or more precursor materials can comprise up to 100% thermoplastic fibers. The fibers in some cases may, therefore, be substantially non-absorbent. In some forms, the nonwoven precursor materials can be either substantially, or completely free, of tow fibers.

The precursor nonwoven materials can comprise any suitable types of fibers. Suitable types of fibers include, but are not limited to: monocomponent, bicomponent, and/or biconstituent, non-round (e.g., shaped fibers (including but not limited to fibers having a trilobal cross-section) and capillary channel fibers). The fibers can be of any suitable size. The fibers may, for example, have major cross-sectional dimensions (e.g., diameter for round fibers) ranging from 0.1-500 microns. Fiber size can also be expressed in denier, which is a unit of weight per length of fiber. The constituent fibers may, for example, range from about 0.1 denier to about 100 denier. The constituent fibers of the nonwoven precursor web(s) may also be a mixture of different fiber types, differing in such features as chemistry (e.g., PE and PP), components (mono- and bi-), shape (i.e. capillary channel and round) and the like.

The nonwoven precursor webs can be formed from many processes, such as, for example, air laying processes, wetlaid processes, meltblowing processes, spunbonding processes, and carding processes. The fibers in the webs can then be bonded via spunlacing processes, hydroentangling, calendar bonding, through-air bonding and resin bonding. Some of such individual nonwoven webs may have bond sites 46 where the fibers are bonded together.

In the case of spunbond webs, the web may have a thermal point bond 46 pattern that is not highly visible to the naked eye. For example, dense thermal point bond patterns are equally and uniformly spaced are typically not highly visible. After the material is processed through the mating male and female rolls, the thermal point bond pattern is still not highly visible. Alternatively, the web may have a thermal point bond pattern that is highly visible to the naked eye. For example, thermal point bonds that are arranged into a macro-pattern, such as a diamond pattern, are more visible to the naked eye. After the material is processed through the mating male and female rolls, the thermal point bond pattern is still highly visible and can provide a secondary visible texture element to the material.

The basis weight of nonwoven materials is usually expressed in grams per square meter (gsm). The basis weight of a single layer nonwoven material can range from about 8 gsm to about 100 gsm, depending on the ultimate use of the material 30. For example, the topsheet of a topsheet/acquisition layer laminate or composite may have a basis weight from about 8 to about 40 gsm, or from about 8 to about 30 gsm, or from about 8 to about 20 gsm. The acquisition layer may have a basis weight from about 10 to about 120 gsm, or from about 10 to about 100 gsm, or from about 10 to about 80 gsm.

The basis weight of a multi-layer material is the combined basis weight of the constituent layers and any other added components. The basis weight of multi-layer materials of interest herein can range from about 20 gsm to about 150 gsm, depending on the ultimate use of the material 30. The nonwoven precursor webs may have a density that is between about 0.01 and about 0.4 g/cm³ measured at 0.3 psi (2 kPa).

The precursor nonwoven webs may have certain desired characteristics. The precursor nonwoven web(s) each have a first surface, a second surface, and a thickness. The first and second surfaces of the precursor nonwoven web(s) may be generally planar. It is typically desirable for the precursor nonwoven web materials to have extensibility to enable the fibers to stretch and/or rearrange into the form of the protrusions. If the nonwoven webs are comprised of two or more layers, it may be desirable for all of the layers to be as extensible as possible. Extensibility is desirable in order to maintain at least some non-broken fibers in the sidewalls around the perimeter of the protrusions. It may be desirable for individual precursor webs, or at least one of the nonwovens within a multi-layer structure, to be capable of undergoing an apparent elongation (strain at the breaking force, where the breaking force is equal to the peak force) of greater than or equal to about one of the following amounts: 100% (that is double its unstretched length), 110%, 120%, or 130% up to about 200%. It is also desirable for the precursor nonwoven webs to be capable of undergoing plastic deformation to ensure that the structure of the deformations is “set” in place so that the nonwoven web will not tend to recover or return to its prior configuration.

Materials that are not extensible enough (e.g., inextensible PP) may form broken fibers around much of the perimeter of the deformation, and create more of a “hanging chad” 90 (i.e., the cap 52 of the protrusions 32 may be at least partially broken from and separated from the rest of the protrusion (as shown in FIG. 20). The area on the sides of the protrusion where the fibers are broken is designated with reference number 91. Materials such as that shown in FIG. 20 will not be suitable for a single layer structure, and, if used, will typically be part of a composite multi-layer structure in which another layer has protrusions 32 as described herein.

When the fibers of a nonwoven web are not very extensible, it may be desirable for the nonwoven to be underbonded as opposed to optimally bonded. A thermally bonded nonwoven web's tensile properties can be modified by changing the bonding temperature. A web can be optimally or ideally bonded, underbonded, or overbonded. Optimally or ideally bonded webs are characterized by the highest breaking force and apparent elongation with a rapid decay in strength after reaching the breaking force. Under strain, bond sites fail and a small amount of fibers pull out of the bond site. Thus, in an optimally bonded nonwoven, the fibers 38 will stretch and break around the bond sites 46 when the nonwoven web is strained beyond a certain point. Often there is a small reduction in fiber diameter in the area surrounding the thermal point bond sites 46. Underbonded webs have a lower breaking force and apparent elongation when compared to optimally bonded webs, with a slow decay in strength after reaching the breaking force. Under strain, some fibers will pull out from the thermal point bond sites 46. Thus, in an underbonded nonwoven, at least some of the fibers 38 can be separated easily from the bond sites 46 to allow the fibers 38 to pull out of the bond sites and rearrange when the material is strained. Overbonded webs also have a lowered breaking force and elongation when compared to optimally bonded webs, with a rapid decay in strength after reaching the breaking force. The bond sites look like films and result in complete bond site failure under strain.

When the nonwoven web comprises two or more layers, the different layers can have the same properties, or any suitable differences in properties relative to each other. In one form, the nonwoven web 30 can comprise a two layer structure that is used in an absorbent article. For convenience, the precursor webs and the material into which they are formed will generally be referred to herein by the same reference numbers. However, in some cases, for additional clarity the precursor web may be designated as 30′. As described above, one of the layers, a second layer 30B, can serve as the topsheet of the absorbent article, and the first layer 30A can be an underlying layer (or sub-layer) and serve as an acquisition layer. The acquisition layer 30A receives liquids that pass through the topsheet and distributes them to underlying absorbent layers. In such a case, the topsheet 30B may be less hydrophilic than sub-layer(s) 30A, which may lead to better dewatering of the topsheet. In other forms, the topsheet can be more hydrophilic than the sub-layer(s). In some cases, the pore size of the acquisition layer may be reduced, for example via using fibers with smaller denier or via increasing the density of the acquisition layer material, to better dewater the pores of the topsheet.

The second nonwoven layer 30B that may serve as the topsheet can have any suitable properties. Properties of interest for the second nonwoven layer, when it serves as a topsheet, in addition to sufficient extensibility and plastic deformation may include uniformity and opacity. As used herein, “uniformity” refers to the macroscopic variability in basis weight of a nonwoven web. As used, herein, “opacity” of nonwoven webs is a measure of the impenetrability of visual light, and is used as visual determination of the relative fiber density on a macroscopic scale. As used herein, “opacity” of the different regions of a single nonwoven deformation is determined by taking a photomicrograph at 20× magnification of the portion of the nonwoven containing the deformation against a black background. Darker areas indicate relatively lower opacity (as well as lower basis weight and lower density) than white areas.

Several examples of nonwoven materials suitable for use as the second nonwoven layer 30B include, but are not limited to: spunbonded nonwovens; carded nonwovens; and other nonwovens with high extensibility (apparent elongation in the ranges set forth above) and sufficient plastic deformation to ensure the structure is set and does not have significant recovery. One suitable nonwoven material as a topsheet for a topsheet/acquisition layer composite structure may be an extensible spunbonded nonwoven comprising polypropylene and polyethylene. The fibers can comprise a blend of polypropylene and polyethylene, or they can be bi-component fibers, such as a sheath-core fiber with polyethylene on the sheath and polypropylene in the core of the fiber. Another suitable material is a bi-component fiber spunbonded nonwoven comprising fibers with a polyethylene sheath and a polyethylene/polypropylene blend core.

The first nonwoven layer 30A that may, for example, serve as the acquisition layer can have any suitable properties. Properties of interest for the first nonwoven layer, in addition to sufficient extensibility and plastic deformation may include uniformity and opacity. If the first nonwoven layer 30A serves as an acquisition layer, its fluid handling properties must also be appropriate for this purpose. Such properties may include: permeability, porosity, capillary pressure, caliper, as well as mechanical properties such as sufficient resistance to compression and resiliency to maintain void volume. Suitable nonwoven materials for the first nonwoven layer when it serves as an acquisition layer include, but are not limited to: spunbonded nonwovens; through-air bonded (“TAB”) carded nonwoven materials; spunlace nonwovens; hydroentangled nonwovens; and, resin bonded carded nonwoven materials. Of course, the composite structure may be inverted and incorporated into an article in which the first layer 30A serves as the topsheet and the second layer 30B serves as an acquisition layer. In such cases, the properties and exemplary methods of the first and second layers described herein may be interchanged.

The layers of a two or more layered nonwoven web structure can be combined together in any suitable manner. In some cases, the layers can be unbonded to each other and held together autogenously (that is, by virtue of the formation of deformations therein). For example, both precursor webs 30A and 30B contribute fibers to deformations in a “nested” relationship that joins the two precursor webs together, forming a multi-layer web without the use or need for adhesives or thermal bonding between the layers. In other forms, the layers can be joined together by other mechanisms. If desired an adhesive between the layers, ultrasonic bonding, chemical bonding, resin or powder bonding, thermal bonding, or bonding at discrete sites using a combination of heat and pressure can be selectively utilized to bond certain regions or all of the precursor webs. In addition, the multiple layers may be bonded during processing, for example, by carding one layer of nonwoven onto a spunbond nonwoven and thermal point bonding the combined layers. In some cases, certain types of bonding between layers may be excluded. For example, the layers of the present structure may be non-hydroentangled together.

If adhesives are used, they can be applied in any suitable manner or pattern including, but not limited to: slots, spirals, spray, and curtain coating. Adhesives can be applied in any suitable amount or basis weight including, but not limited to between about 0.5 and about 30 gsm, alternatively between about 2 and about 5 gsm. Examples of adhesives could include hot melt adhesives, such as polyolefins and styrene block copolymers.

A certain level of adhesive may reduce the level of fuzz on the surface of the nonwoven material even though there may be a high percentage of broken fibers as a result of the deformation process. Glued dual-layer laminates produced as described herein are evaluated for fuzz. The method utilizes a Martindale Abrasion Tester, based upon ASTM D4966-98. After abrading the samples, they are graded on a scale of 1-10 based on the degree of fiber pilling (1=no fiber pills; 10 =large quantity and size of fiber pills). The protrusions are oriented away from the abrader so the land area in between the depressions is the primary surface abraded. Even though the samples may have a significant amount of fiber breakage (greater than 25%, sometimes greater than 50%) in the side walls of the protrusions/depressions, the fuzz value may be low (around 2) for several different material combinations, as long as the layers do not delaminate during abrasion. Delamination is best prevented by glue basis weight, for example a glue basis weight greater than 3 gsm, and glue coverage.

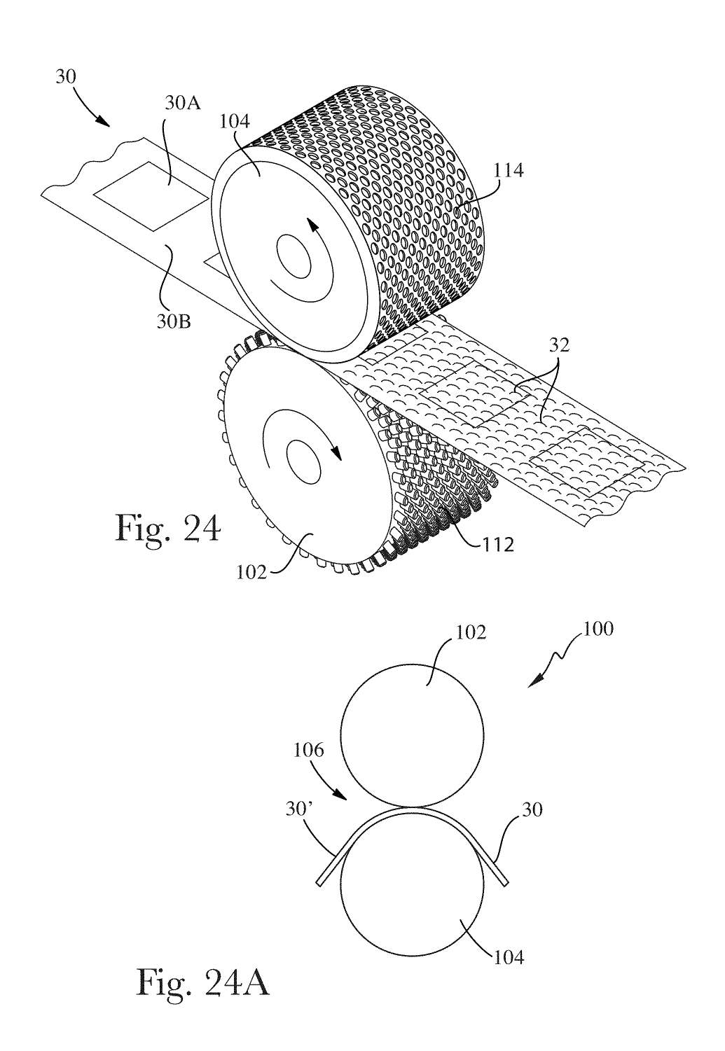

When the precursor nonwoven web comprises two or more layers, it may be desirable for at least one of the layers to be continuous, such as in the form of a web that is unwound from a roll. In some forms, each of the layers can be continuous. In alternative forms, such as shown in FIG. 24, one or more of the layers can be continuous, and one or more of the layers can have a discrete length. The layers may also have different widths. For example, in making a combined topsheet and acquisition layer for an absorbent article, the nonwoven layer that will serve as the topsheet may be a continuous web, and the nonwoven layer that will serve as the acquisition layer may be fed into the manufacturing line in the form of discrete length (for example, rectangular, or other shaped) pieces that are placed on top of the continuous web. Such an acquisition layer may, for example, have a lesser width than the topsheet layer. The layers may be combined together as described above.

### III. Methods of Making the Nonwoven Materials

The nonwoven materials are made by a method comprising the steps of: a) providing at least one precursor nonwoven web; b) providing an apparatus comprising a pair of forming members comprising a first forming member (a “male” forming member) and a second forming member (a “female” forming member); and c) placing the precursor nonwoven web(s) between the forming members and mechanically deforming the precursor nonwoven web(s) with the forming members. The forming members have a machine direction (MD) orientation and a cross-machine direction (CD) orientation.

The first and second forming members can be plates, rolls, belts, or any other suitable types of forming members. In some forms, it may be desirable to modify the apparatus for incrementally stretching a web described in U.S. Pat. No. 8,021,591, Curro, et al. entitled “Method and Apparatus for Incrementally Stretching a Web” by providing the activation members described therein with the forming elements of the type described herein. In the form of the apparatus 100 shown in FIG. 21, the first and second forming members 102 and 104 are in the form of non-deformable, meshing, counter-rotating rolls that form a nip 106 therebetween. The precursor web(s) is/are fed into the nip 106 between the rolls 102 and 104. Although the space between the rolls 102 and 104 is described herein as a nip, as discussed in greater detail below, in some cases, it may be desirable to avoid compressing the precursor web(s) to the extent possible.

### First Forming Member

The first forming member (such as “male roll”) 102 has a surface comprising a plurality of first forming elements which comprise discrete, spaced apart male forming elements 112. The male forming elements are spaced apart in the machine direction and in the cross-machine direction. The term “discrete” does not include continuous or non-discrete forming elements such as the ridges and grooves on corrugated rolls (or “ring rolls”) which have ridges that may be spaced apart in one, but not both, of the machine direction and in the cross-machine direction.

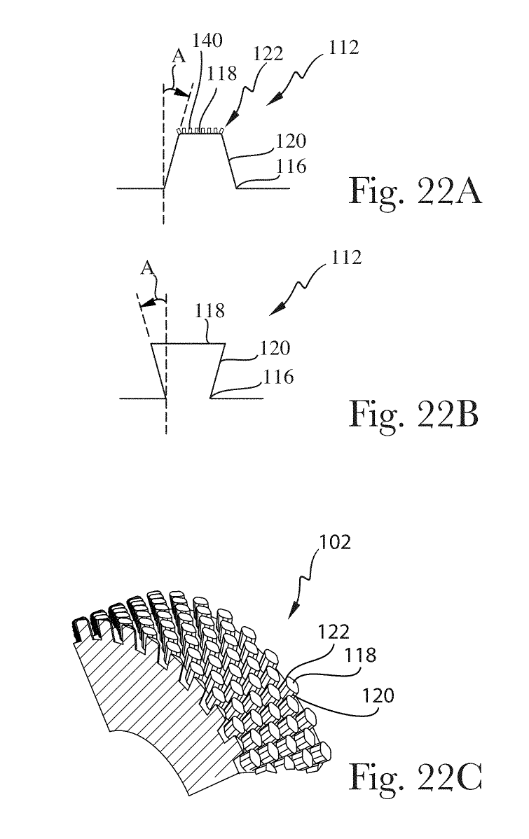

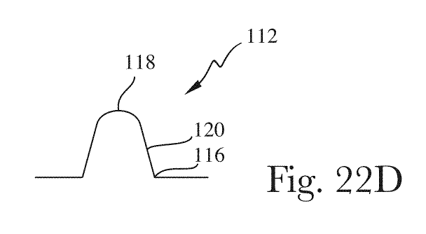

As shown in FIG. 22, the male forming elements 112 have a base 116 that is joined to (in this case is integral with) the first forming member 102, a top 118 that is spaced away from the base, and side walls (or “sides”) 120 that extend between the base 116 and the top 118 of the male forming elements. The male elements 112 may also have a transition portion or region 122 between the top 118 and the side walls 120. The male elements 112 also have a plan view periphery, and a height Hi (the latter being measured from the base 116 to the top 118). The discrete elements on the male roll may have a top 118 with a relatively large surface area (e.g., from about 1 mm to about 10 mm in width, and from about 1 mm to about 20 mm in length) for creating a wide deformation. The male elements 112 may, thus, have a plan view aspect ratio (ratio of length to width) that ranges from about 1:1 to about 10:1. For the purpose of determining the aspect ratio, the larger dimension of the male elements 112 will be consider the length, and the dimension perpendicular thereto will be considered to be the width of the male element. The male elements 112 may have any suitable configuration. The base 116 and the top 118 of the male elements 112 may have any suitable plan view configuration, including but not limited to: a rounded diamond configuration as shown in FIGS. 21 and 22, an American football-like shape, triangle, circle, clover, a heart-shape, teardrop, oval, or an elliptical shape. The configuration of the base 116 and the configuration of the top 118 of the male elements 112 may be in any of the following relationships to each other: the same, similar, or different. The top 118 of the male elements 112 can be flat, rounded, or any configuration therebetween.

The side walls 120 of the male elements 112 may have any suitable configuration. The male elements 112 may have vertical side walls 120, or tapered side walls 120. By vertical side walls, it is meant that the side walls 120 have zero degree side wall angles relative to the perpendicular from the base 116 of the side wall. In other forms, as shown in FIG. 22A, the side walls 120 can be tapered inwardly toward the center of the male forming elements 112 from the base 116 to the top 118 so that the side walls 120 form an angle, A, greater than zero. In still other forms, as shown in FIG. 22B, the male forming elements 112 may have a wider top surface than base so that the side walls 120 are angled outwardly away from the center of the male forming elements 112 from the base 116 to the top 118 of the male elements 112 (that is, the side walls may be undercut). The side wall angle can be the same on all sides of the male elements 112. Alternatively, the male elements 112 may have a different side wall angle on one or more of their sides. For example, the leading edge (or “LE”) and trailing edge (or “TE”) of the male elements (with respect to the machine direction) may have equal side wall angles, and the sides of the male elements may have equal side wall angles, but the side wall angles of the LE and TE may be different from the side wall angle of the sides. In certain forms, for example, the side wall angle of the sides of the male elements 112 may be vertical, and the side walls of the LE and TE may be slightly undercut.

The transition region or “transition” 122 between the top 118 and the side walls 120 of the male elements 112 may also be of any suitable configuration. The transition 122 can be in the form of a sharp edge (as shown in FIG. 22C) in which case there is zero, or a minimal radius where the side walls 120 and the top 118 of the male elements meet. That is, the transition 122 may be substantially angular, sharp, non-radiused, or non-rounded. In other forms, such as shown in FIG. 22, the transition 122 between the top 118 and the side walls 120 of the male elements 112 can be radiused, or alternatively beveled. Suitable radiuses include, but are not limited to: zero (that is, the transition forms a sharp edge), 0.01 inch (about 0.25 mm), 0.02 inch (about 0.5 mm), 0.03 inch (about 0.76 mm), 0.04 inch (about 1 mm) (or any 0.01 inch increment above 0.01 inch), up to a fully rounded male element as shown in FIG. 22D.

Numerous other forms of the male forming elements 112 are possible. In other forms, the top 118 of the male elements 112 can be of different shapes from those shown in the drawings. In other forms, the male forming elements 112 can be disposed in other orientations on the first forming member 102 rather than having their length oriented in the machine direction (including CD-orientations, and orientations between the MD and CD). The male forming elements 112 on the first forming member 102 may, but need not, all have the same configuration or properties. In certain forms, the first forming member 102 can comprise some male forming elements 112 having one configuration and/or properties, and other male forming elements 112 having one or more different configurations and/or properties.

The method of making the nonwoven materials may be run with the first forming member 102 and male elements 112 under any of the following conditions: at room temperature; with a chilled first forming member 102 and/or male elements 112; or with heated first forming member and/or male elements. In some cases, it may be desired to avoid heating the first forming member 102 and/or male elements 112. It may be desirable to avoid heating the first forming member and/or the male elements altogether. Alternatively, it may be desirable to avoid heating the first forming member and/or the male elements to a temperature at or above that which would cause the fibers of the nonwoven to fuse together. In some cases, it may be desirable to avoid heating the first forming member and/or the male elements to a temperature that is greater than or equal to any of the following temperatures: 130° C., 110° C., 60° C., or greater than 25° C.

### Second Forming Member

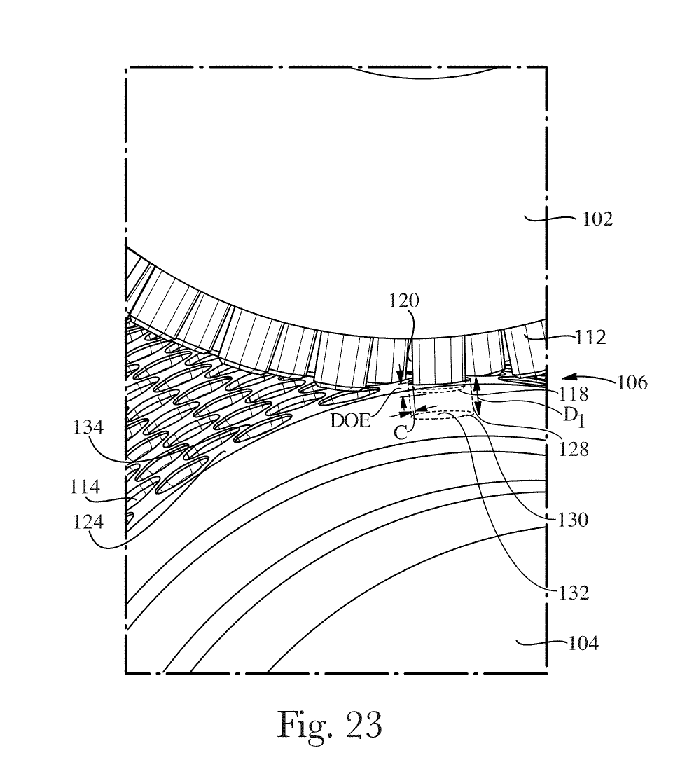

As shown in FIG. 21, the second forming member (such as “female roll”) 104 has a surface 124 having a plurality of cavities or recesses 114 therein. The recesses 114 are aligned and configured to receive the male forming elements 112 therein. Thus, the male forming elements 112 mate with the recesses 114 so that a single male forming element 112 fits within the periphery of a single recess 114, and at least partially within the recess 114 in the z-direction. The recesses 114 have a plan view periphery 126 that is larger than the plan view periphery of the male elements 112. As a result, the recess 114 on the female roll may completely encompass the discrete male element 112 when the rolls 102 and 104 are intermeshed. The recesses 114 have a depth Di shown in FIG. 23. In some cases, the depth Di of the recesses may be greater than the height Hi of the male forming elements 112.

The recesses 114 have a plan view configuration, side walls 128, a top edge or rim 134 around the upper portion of the recess where the side walls 128 meet the surface 124 of the second forming member 104, and a bottom edge 130 around the bottom 132 of the recesses where the side walls 128 meet the bottom 132 of the recesses.

The recesses 114 may have any suitable plan view configuration provided that the recesses can receive the male elements 112 therein. The recesses 114 may have a similar plan view configuration as the male elements 112. In other cases, some or all of the recesses 114 may have a different plan view configuration from the male elements 112.

The side walls 128 of the recesses 114 may be oriented at any suitable angle. In some cases, the side walls 128 of the recesses may be vertical. In other cases, the side walls 128 of the recesses may be oriented at an angle. Typically, this will be an angle that is tapered inwardly from the top 134 of the recess 114 to the bottom 132 of the recess. The angle of the side walls 128 of the recesses can, in some cases, be the same as the angle of the side walls 120 of the male elements 112. In other cases, the angle of the side walls 128 of the recesses can differ from the angle of the side walls 120 of the male elements 112.

The top edge or rim 134 around the upper portion of the recess where the side walls 128 meet the surface 124 of the second forming member 104 may have any suitable configuration. The rim 134 can be in the form of a sharp edge (as shown in FIG. 23) in which case there is zero, or a minimal radius where the side walls 128 of the recesses meet the surface of the second forming member 104. That is, the rim 134 may be substantially angular, sharp, non-radiused, or non-rounded. In other forms, the rim 134 can be radiused, or alternatively beveled. Suitable radiuses include, but are not limited to: zero (that is, form a sharp edge), 0.01 inch (about 0.25 mm), 0.02 inch (about 0.5 mm), 0.03 inch (about 0.76 mm), 0.04 inch (about 1 mm) (or any 0.01 inch increment above 0.01 inch) up to a fully rounded land area between some or all of the side walls 128 around each recess 114. The bottom edge 130 of the recesses 114 may be sharp or rounded.

As discussed above, the recesses 114 may be deeper than the height Hi of the male elements 112 so the nonwoven material is not nipped (or squeezed) between the male and female rolls 102 and 104 to the extent possible. However, it is understood that passing the precursor web(s) between two rolls with a relatively small space therebetween will likely apply some shear and compressive forces to the web(s). The present method, however, differs from some embossing processes in which the top of the male elements compress the material to be embossed against the bottom of the female elements, thereby increasing the density of the region in which the material is compressed.

The depth of engagement (DOE) is a measure of the level of intermeshing of the forming members. As shown in FIG. 23, the DOE is measured from the top 118 of the male elements 112 to the (outermost) surface 124 of the female forming member 114 (e.g., the roll with recesses). The DOE should be sufficiently high, when combined with extensible nonwoven materials, to create protrusions 32 having a distal portion or cap 52 with a maximum width that is greater than the width of the base opening 44. The DOE may, for example, range from at least about 1.5 mm, or less, to about 5 mm, or more. In certain forms, the DOE may be between about 2.5 mm to about 5 mm, alternatively between about 3 mm and about 4 mm. The formation of protrusions 32 having a distal portion with a maximum width that is greater than the width of the base opening 44 is believed to differ from most embossing processes in which the embossments typically take the configuration of the embossing elements, which have a base opening that is wider than the remainder of the embossments.

As shown in FIG. 23, there is a clearance, C, between the sides 120 of the male elements 112 and the sides (or side walls) 128 of the recesses 114. The clearances and the DOE's are related such that larger clearances can permit higher DOE's to be used. The clearance, C, between the male and female roll may be the same, or it may vary around the perimeter of the male element 112. For example, the forming members can be designed so that there is less clearance between the sides of the male elements 112 and the adjacent side walls 128 of the recesses 114 than there is between the side walls at the end of the male elements 112 and the adjacent side walls of the recesses 114. In other cases, the forming members can be designed so that there is more clearance between the sides 120 of the male elements 112 and the adjacent side walls 128 of the recesses 114 than there is between the side walls at the end of the male elements 112 and the adjacent side walls of the recesses. In still other cases, there could be more clearance between between the side wall on one side of a male element 112 and the adjacent side wall of the recess 114 than there is between the side wall on the opposing side of the same male element 112 and the adjacent side wall of the recess. For example, there can be a different clearance at each end of a male element 112; and/or a different clearance on each side of a male element 112. Clearances can range from about 0.005 inches (about 0.1 mm) to about 0.1 inches (about 2.5 mm).

Some of the aforementioned male element 112 configurations alone, or in conjunction with the second forming member 104 and/or recess 114 configurations may provide additional advantages. This may be due to by greater lock of the nonwoven material on the male elements 112, which may result in more uniform and controlled strain on the nonwoven precursor material. This may produce more well-defined protrusions 32 and a stronger visual signal for consumers, giving the appearance of softness, absorbency, and/or dryness.

The precursor nonwoven web 30 is placed between the forming members 102 and 104. The precursor nonwoven web can be placed between the forming members with either side of the precursor web (first surface 34 or second surface 36) facing the first forming member, male forming member 102. For convenience of description, the second surface 36 of the precursor nonwoven web will be described herein as being placed in contact with the first forming member 102. (Of course, in other forms, the second surface 36 of the precursor nonwoven web can be placed in contact with the second forming member 104.)

The precursor material is mechanically deformed with the forming members 102 and 104 when a force is applied on the nonwoven web with the forming members 102 and 104. The force can be applied in any suitable manner. If the forming members 102 and 104 are in the form of plates, the force will be applied when the plates are brought together. If the forming members 102 and 104 are in the form of counter-rotating rolls (or belts, or any combination of rolls and belts), the force will be applied when the precursor nonwoven web passes through the nip between the counter-rotating elements. The force applied by the forming members impacts the precursor web and mechanically deforms the precursor nonwoven web.

Numerous additional processing parameters are possible. If desired, the precursor nonwoven web may be heated before it is placed between the forming members 102 and 104. If the precursor nonwoven web is a multi-layer structure, any layer or layers of the same can be heated before the layers are combined. Alternatively, the entire multi-layer nonwoven web can be heated before it is placed between the forming members 102 and 104. The precursor nonwoven web, or layer(s) of the same, can be heated in any suitable manner including, but not limited to using conductive heating (such as by bringing the web(s) in contact with heated rolls), or by convective heating (i.e., by passing the same under a hot air knife or through an oven). The heating should be non-targeted, and without the help of any agent. The first forming member 102 and/or second forming member 104 (or any suitable portion thereof) can also be heated. If desired, the web could be additionally, or alternatively, heated after it is mechanically deformed.

If the precursor material is fed between forming members comprising counter-rotating rolls, several processing parameters may be desirable. With regard to the speed at which the precursor web is fed between the counter-rotating rolls, it may be desirable to overfeed the web (create a negative draw) going into the nip 106 between the rolls. The surface speed of the metering roll immediately upstream of the forming members 102 and 104 may be between about 1 and 1.2 times the surface speed of the forming members 102 and 104. It may be desirable for the tension on the precursor web immediately before forming members 102 and 104 to be less than about 5 lbs. force (about 22 N), alternatively less than about 2 lbs. force (about 9 N) for a web width of 0.17 m. With regard to the speed at which the deformed web 30 is removed from between the counter-rotating rolls, it may be desirable to create a positive draw coming out of the nip between the rolls. The surface speed of the metering roll immediately downstream of the forming members 102 and 104 may be between about 1 and 1.2 times the surface speed of the forming members 102 and 104. It may be desirable for the tension on the web immediately after the forming members 102 and 104 to be less than about 5 lbs. force (about 22 N), alternatively less than about 2 lbs. force (about 9 N).

As shown in FIG. 24A, rather than feeding the precursor web 30′ into the nip 106 between the forming members 102 and 104 without the precursor web 30′ contacting any portion of the forming members prior to or after the nip, it may be desirable for the web to pre-wrap the second forming member 104 prior to entering the nip 106, and for the web 30 to post wrap second forming member 104 after passing through the nip.

The apparatus 100 for deforming the web can comprise multiple nips for deforming portions of the web in the same location such as described in U.S. Patent Publication No. U.S. 2012/0064298 A1, Orr, et al. For example, the apparatus may comprise a central roll and satellite rolls with equal DOE or progressively greater DOE with each successive roll. This can provide benefits such as reducing damage to the web and/or helping to further ensure that the deformations are permanently set in the web thereby preventing the web from recovering toward its undeformed condition.

The apparatus for deforming the web can also comprise belts, or other mechanisms, for holding down the longitudinal edges of the web to prevent the web from being drawn inward in the cross-machine direction.

When deforming multiple webs that are laminated together with an adhesive, it may be desirable to chill the forming members in order to avoid glue sticking to and fouling the forming members. The forming members can be chilled using processes know in the art. One such process could be an industrial chiller that utilizes a coolant, such as propylene glycol. In some cases, it may be desirable to operate the process in a humid environment such that a layer of condensate forms on the forming members.

The apparatus 100 for deforming the web can be at any suitable location in any suitable process. For example, the apparatus can be located in-line with a nonwoven web making process or a nonwoven laminate making process. Alternatively, the apparatus 100 can be located in-line in an absorbent article converting process (such as after the precursor web is unwound and before it is incorporated as part of the absorbent article).

The process forms a nonwoven web 30 comprising a generally planar first region 40 and a plurality of discrete integral second regions 42 that comprise deformations comprising protrusions 32 extending outward from the first surface 34 of the nonwoven web and openings in the second surface 36 of the nonwoven web. (Of course, if the second surface 36 of the precursor nonwoven web is placed in contact with the second forming member 104, the protrusions will extend outward from the second surface of the nonwoven web and the openings will be formed in the first surface of the nonwoven web.) Without wishing to be bound by any particular theory, it is believed that the extensibility of the precursor web (or at least one of the layers of the same) when pushed by the male forming elements 112 into the recesses 114 with depth of engagement DOE being less than the depth Di of the recesses, stretches a portion of the nonwoven web to form a deformation comprising a protrusion with the enlarged cap and wide base opening described above. (This can be analogized to sticking one's finger into an uninflated balloon to stretch and permanently deform the material of the balloon.)

In cases in which the precursor nonwoven material 30′ comprises more than one layer, and one of the layers is in the form of discrete pieces of nonwoven material, as shown in FIG. 24, it may be desirable for the deformations to be formed so that the base openings 44 are in the continuous layer (such as 30B) and the protrusions 32 extend toward the discrete layer (such as 30A). Of course, in other forms, the deformations in such a structure can be in the opposite orientation. The deformations can be distributed in any suitable manner over the surfaces of such continuous and discrete layers. For example, the deformations can: be distributed over the full length and/or width of the continuous layer; be distributed in an area narrower than the width of the continuous layer; or be limited to the area of the discrete layer.

In some instances, the ratio of the circumference of the protrusions (loop circumference length) to the length of the second surface opening 64 (see FIG. 11) is less than 4:1. To measure the loop circumference length, arrange the web comprising the protrusion so that the viewing direction is co-linear with the longitudinal axis (MD) of the protrusion. Adjust the magnification so that one protrusion is completely in view. If necessary, a cross-section of the protrusion may be obtained by cutting the protrusion perpendicular to the longitudinal axis using sharp scissors or a razor blade, taking care in preserving the overall geometry of the protrusion while cutting it. Referring to FIG. 12, measure and record the loop circumference length by starting the measurement at the first origination point A, proceeding along the median path of the loop fibers B, and terminating the measurement at the second origination point C. Measure and record the base length of the second surface opening 64, parallel to the plane of the web between the first origination point A and the second origination point C. The loop base length of the second surface opening 64 is measured parallel to the plane of the web and may be at the plane of the web or above the plane of the web. The protrusions are measured where the protrusions are not under any pressure or strain.

### IV. Non-Uniform Interruptions

As described herein, with respect to FIGS. 26 and 27, an absorbent article may comprise the nonwoven material laminate 30 comprising the topsheet 84 and the acquisition layer 86. The topsheet may comprise cotton fibers and may be hydrophobic. In other instances, the absorbent article may comprise a laminate comprising a film topsheet 84 and a nonwoven acquisition layer 86 or a nonwoven secondary topsheet. The topsheet 84 and the acquisition layer 86 may be nested together as described herein to form a continuous generally planar first region 40 and a plurality of discrete integral second regions 42. The plurality of discrete integral second regions 42 may extend toward the absorbent core 88 (FIG. 26) or may extend away from the absorbent core 88 (FIG. 27). The absorbent core 88 may be positioned at least partially between the nonwoven material laminate 30 and the backsheet 94. The absorbent core 88 may comprise an absorbent material 89 enclosed in a core bag 91. The absorbent material 89 may be free of, or substantially free of (e.g., less than 5% by weight), air-felt (e.g., cellulosic fibers), with the remainder of the absorbent material being superabsorbent polymers. In other instances, the absorbent material 89 may comprise a mixture of superabsorbent polymers and air-felt in any suitable ratio.

In some instances, a carrier layer may be provided intermediate the acquisition layer 86 and a distribution layer 92. The carrier layer may be a generally planar nonwoven material used to essentially “carry” or be joined to the distribution layer 92, in the event that the distribution layer 92 is not capable of adequate bonding to the garment-facing surface of the laminate owing to its three-dimensional structure. The distribution layer 92 may comprise cross-linked cellulosic fibers, other cellulosic or pulp fibers, and/or one or more nonwoven materials. The distribution layer 92 may be joined to the carrier layer to form a distribution layer/carrier layer laminate. In some forms, the distribution layer 92 may be optional.

The topsheet 84 and the acquisition layer 86 may both comprise non-apertured nonwoven materials. Non-apertured means that no predetermined apertures were intended in the nonwoven materials (unlike a typical apertured topsheet). Variances in the nonwoven material are not apertures and are within the scope of the term non-apertured. Non-apertured materials may have one or more non-uniform interruptions. The non-uniform interruptions may be formed when the topsheet 84 and the acquisition layer 86 are brought together and run through the process of FIG. 21, for example. The topsheet 84 may be hydrophobic and the acquisition layer 86 may be hydrophilic. In other instances, the topsheet 84 may be less hydrophilic than the acquisition layer 86.

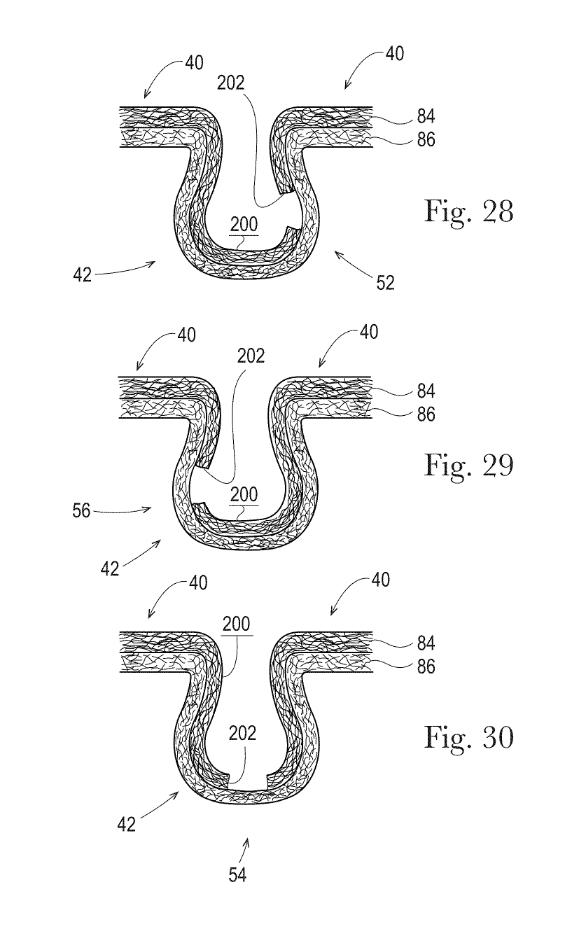

Referring to FIG. 28-30, a series of example cross-sectional illustrations of discrete integral second regions 42 with an adjacent generally planar first region 40 are illustrated. The discrete integral second regions 42 may be nested areas in the topsheet 84 and the acquisition layer 86 with the generally planar first region 40 also being formed by the topsheet 84 and the acquisition layer 86 in non-nested areas. The discrete integral second regions 42 each comprise a wearer-facing surface 200. One or more non-uniform interruptions 202 may be present in the topsheet 84 in the discrete integral second regions 42. These non-uniform interruptions 202 may be generally the same as that described with respect to FIGS. 15C-15F, but may now be in the topsheet 84, and generally are not in the acquisition layer 86, but may be in some instances. The non-uniform interruptions 202 may be in the caps 52 (FIG. 28), the sidewalls 56 (FIG. 29), and/or the distal ends 54 (FIG. 30) of the discrete integral second regions 42. In other instances, two or more non-uniform interruptions 202 may be present in a certain discrete integral second region 42. The non-uniform interruptions 200 in the topsheet 84 may be formed when the nonwoven material laminate 30 is run through the process of FIG. 21, or may be formed in other manners. A non-uniform interruption 202 in the topsheet 84 may not be formed in all of the discrete integral second regions 42, but may be formed in at least some of the discrete integral second regions 42. In some instances, the non-uniform interruptions 202 in the topsheet 84 may be formed in all of the plurality of discrete integral second regions 42. The non-uniform interruptions 202 may be randomly positioned in the topsheet 84 in the discrete integral second regions 42. Stated another way, the non-uniform interruptions 202 may be positioned at different locations in different discrete integral second regions 42 depending on where tearing occurs in the topsheet 84 during creation of the three-dimensional structure.

In an instance, the topsheet 84 may be less extensible than the acquisition layer 86 to allow for non-uniform interruption creation in the topsheet 84. The topsheet 84 may comprise nano-fibers (diameters of less than one micron). Nano fibers in the topsheet 84 may improve masking of bodily exudates below the topsheet.

Still referring to FIGS. 28-30, the acquisition layer 86 may form portions of the wearer-facing surface 200 of the discrete integral second regions 42 in the non-uniform interruptions 202. Stated another way, a portion of the acquisition layer 86 underlying the non-uniform interruptions 202 is directly exposed to bodily exudates entering the discrete integral second regions 42. By having a hydrophobic topsheet 86, or a topsheet that is less hydrophilic than the acquisition layer 86, bodily exudates may be directed to and through the non-uniform interruptions 202 and thereby wicked into the absorbent article by the absorbent core. Although the discrete integral second regions 42 are illustrated in FIGS. 28-30 extending toward an absorbent core 88 (see FIG. 26), they may also extend away from the absorbent core 88 (see FIG. 27).

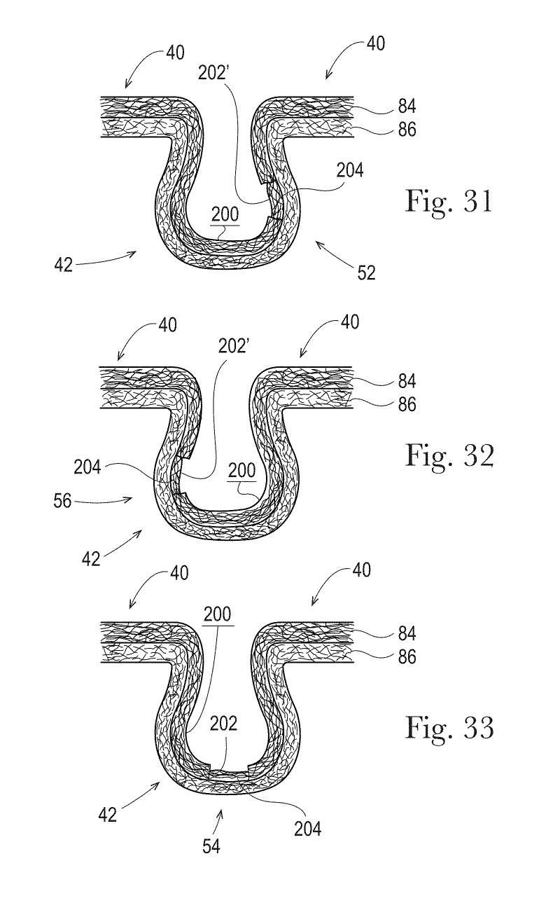

Referring to FIGS. 31-33, a series of example cross-sectional illustrations of discrete integral second regions 42 with an adjacent generally planar first region are illustrated. In this instance, it is illustrated how non-uniform interruptions 202′ may be areas of low fiber density. Stated another way, fibers 204, or portions thereof, may extend through the non-uniform interruptions 202′ creating areas in the topsheet 84 having low fiber density compared to other areas of the topsheet 84. The acquisition layer 86 may form portions of the wearer-facing surface 200 in the non-uniform interruptions 202′, thereby providing the benefits described above. The non-uniform interruptions 202′ may be randomly positioned, and may have any other features, as described above with respect to the non-uniform interruptions 202. Although the discrete integral second regions 42 are illustrated in FIGS. 31-33 extending toward an absorbent core 88 (see FIG. 26), they may also extend away from the absorbent core 88 (see FIG. 27).

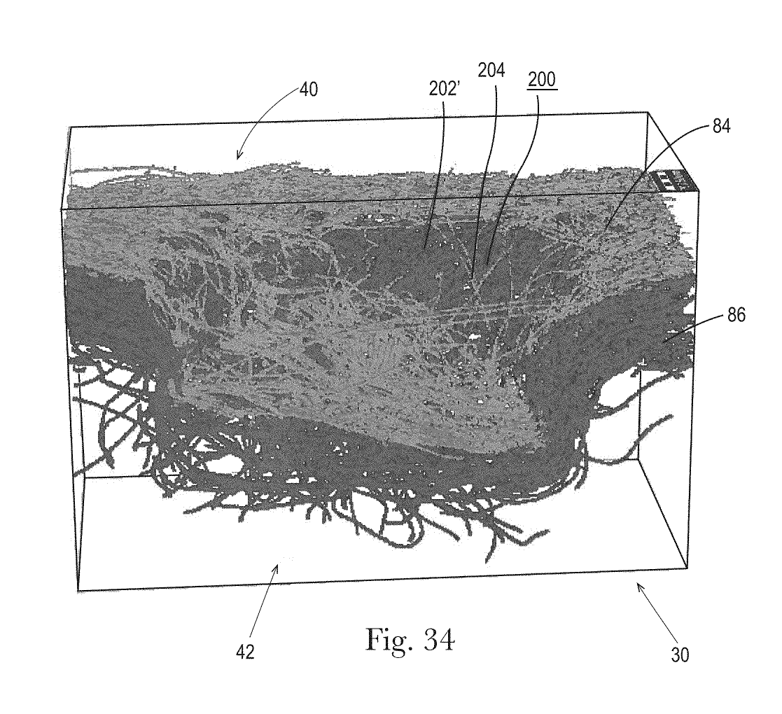

Referring to FIG. 34, a schematic rendering of a portion of the nonwoven material laminate 30 is illustrated. The lighter gray material is a topsheet 84 and the darker gray material is an acquisition layer 86. A non-uniform interruption 202′ (a low fiber density area) is illustrated in the discrete integral second region 42. It can been seen how portion of the acquisition layer 86 forms part of the wearer-facing surface 200 of the discrete integral second region 42 within the non-uniform interruption 202′.

The structures described with respect to FIGS. 28-34 may aid in reducing rewet, owing to the hydrophobic topsheet 84 (or less hydrophilic topsheet 84 than the acquisition layer 86) without apertures in the generally planar first region 40. Further, acquisition speed may be significantly increased owing to the hydrophilic acquisition layer forming portion of the wearer-facing surface 200 in the discrete integral second projections 42 and the void volume capacity of the discrete integral second regions 42. Softness benefits and reduced skin red-marking benefits may also be achieved by the structures of FIGS. 28-34, since apertures are not present in any surface (i.e., generally planar first region) that contacts the wearer (at least when the discrete integral second regions 42 extend towards the absorbent core 88).

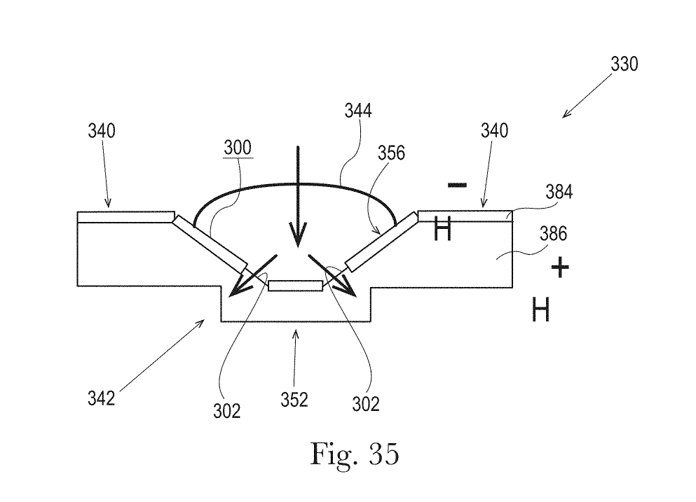

Referring to FIG. 35, a schematic cross-sectional example of a second type of nonwoven material laminate 330 is illustrated. The nonwoven material laminate 330 comprises a topsheet 384 and an acquisition layer 386. In other instances, the absorbent article may comprise a laminate comprising a film topsheet and a nonwoven acquisition layer 386 or a nonwoven secondary topsheet. The nonwoven material laminate 330 may be used in an absorbent article as described above with respect to FIGS. 26-34 instead of the nonwoven material laminate 30. The nonwoven material laminate 330 may also be used with distribution layer 96 as described above with reference to FIGS. 26 and 27. The H− symbol for the topsheet 384 means that the topsheet 384 is either hydrophobic or is less hydrophilic than the acquisition layer 386, which is associated with the H+ symbol. The nonwoven material laminate 330 may comprise a generally planar continuous land area 340 and a plurality of three-dimensional deformations 342. Two non-uniform interruptions 302 are illustrated in the topsheet 384 in the three-dimensional deformation 342 in FIG. 35, although one or more may be provided. The non-uniform interruptions 302 may be areas of the topsheet 384 with no fibers (e.g. non-uniform interruptions 202), or areas of the topsheet 384 with low fiber density, as described above with respect to the non-uniform interruptions 202′.

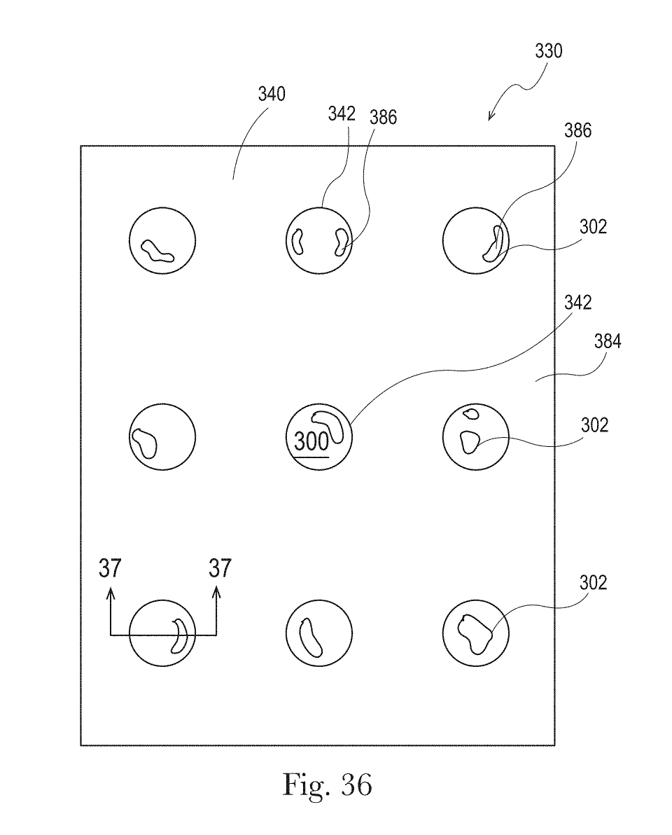

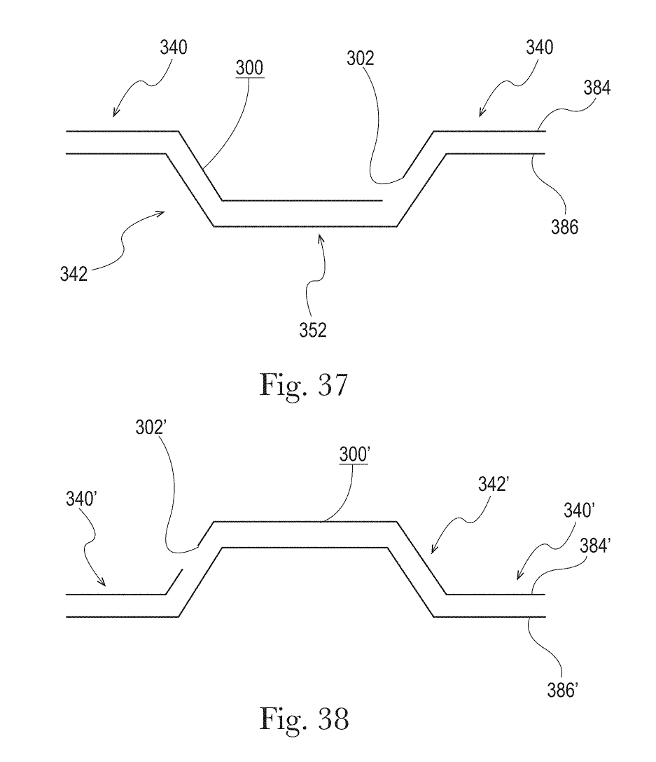

FIG. 36 is a top view of a wearer-facing surface of a nonwoven material laminate 330 with a generally planar continuous land area 340 and a plurality of three-dimensional deformations 342. FIG. 37 is a cross-sectional view of the area indicated by line 37-37 of FIG. 36. Although all of the three-dimensional deformations 342 are illustrated as having non-uniform interruptions 302 in the topsheet 384, only some of the three-dimensional deformations 342 may have the non-uniform interruptions 302 in the topsheet 384. The non-uniform interruptions 302 in the topsheet 384 may be at random locations within the three-dimensional deformations 342, such as in the side walls 356, at the distal ends 352, or both, for example.

By providing the non-uniform interruptions 302 in the topsheet 384 within the three-dimensional deformations 342, a wearer-facing surface 300 of the three-dimensional deformations 342 may comprise a portion of the hydrophilic acquisition layer 386 in areas underlying the non-uniform interruptions. By providing exposed areas of the hydrophilic acquisition layer 386 on the wearer-facing surface 300 of the three-dimensional deformations 342, bodily exudates 344 may be quickly acquired by the acquisition layer and wicked towards the absorbent core. Other contact angles and configurations are also within the scope of the present disclosure. Having a topsheet 384 that is hydrophobic, or that is less hydrophilic than the hydrophilic acquisition layer 384, aids this acquisition process as it channels the bodily exudates 344 into the three-dimensional deformations 342 where they can then “drain” through the non-uniform interruptions 302 in the topsheet 384. The conical shape of upper portions of the wearer-facing surface 300 of the side walls 356 of the three-dimensional deformations 342 also aids in absorbing bodily exudates within the three-dimensional deformations 342 and channeling them through the non-uniform interruptions 302. The hydrophobic topsheet 384, or the topsheet that is less hydrophilic than the acquisition layer 386 also aids in rewet.

Although, the three-dimensional deformations 342 are illustrated in FIGS. 35-37 as extending toward the absorbent core, they may also extend away from the absorbent core. A schematic cross-sectional example of a nonwoven material laminate 330′ with three-dimensional deformations 342′ extending away from the absorbent core is illustrated in FIG. 38. Like elements as FIG. 37 are numbered with primes in FIG. 38.

When the three-dimensional features are facing toward the absorbent core (e.g., FIG. 26), an example distance from an edge of one three-dimensional deformation (e.g., 342) or one discrete integral second region (e.g., 42—FIG. 26) and the closest edge of a neighboring three-dimensional deformation (e.g., 342) or discrete integral second region (e.g., 42—FIG. 26), respectively, may be between about 1 mm to about 200 mm, about 1 mm to about 100 mm, about 1 mm to about 50 mm, about 1 mm to about 10 mm, or about 2 mm to about 5 mm, specifically reciting all 0.1 mm increments within the specified ranges and all ranges formed therein or thereby. The “edge” may be where the three-dimensional deformation (e.g., 342) or one discrete integral second region (e.g., 42) transitions into the land areas (e.g., 40, 340).

When the three-dimensional features are facing away from the absorbent core (e.g., FIG. 27), an example distance from a side wall of one three-dimensional deformation (e.g., 342′) or one discrete integral second region (e.g., 42—FIG. 27) and the closest side wall of a neighboring three-dimensional deformation (e.g., 342′) may be between about 1 mm to about 200 mm, about 1 mm to about 100 mm, about 1 mm to about 50 mm, about 1 mm to about 10 mm, or about 2 mm to about 5 mm, specifically reciting all 0.1 mm increments within the specified ranges and all ranges formed therein or thereby.

The typical three-dimensional deformation Opening Dimension is between about 0.5 mm and about 10 mm, about 1 mm to about 5 mm, or about 1 mm to about 3 mm, specifically reciting all 0.1 mm increments with the specified ranges and all ranges formed therein or thereby, according to the Opening Dimension Test herein.

The non-uniform interruptions (e.g., 202, 202′, 302, 302′) in the topsheet discussed herein may be positioned in the range of about 100 microns to about 2000 microns, about 100 microns to about 1000 microns, or about 100 microns to about 500 microns, from the wearer-facing surface of the first generally planar region (e.g., 40) or the land areas (e.g., 340), specifically reciting all 1 micron increments with in the specified ranges and all ranges formed therein or thereby.

The topsheet may have smaller average diameter of fibers than the acquisition layer in order to provide improved softness and masking/opacity of the absorbent article. Fibers of the topsheet may have an average diameter between about 0.2 microns and about 30 microns or between about 0.2 microns and about 20 microns, specifically reciting all 0.01 micron increments within the specified ranges and all ranges formed therein or thereby. Fibers of the acquisition layer may have an average diameter between about 5 and about 50 microns, between about 20 and about 40 microns, or between about 20 microns and about 30 microns, specifically reciting all 0.01 micron increments within the specified ranges and all ranges formed therein or thereby. Most related art has larger average diameters of fibers in the topsheet and smaller average diameters of fibers in the acquisition layer to create a capillary gradient. All average diameters for fibers are measured according to the Fiber Diameter and Denier Test herein.

The topsheet may have less extensibility than the acquisition layer to maximize non-uniform interruptions in the topsheet in the three-dimensional deformations.

The depth of engagement (DOE) to create the non-uniform interruptions may be chosen in order to maximize the non-uniform interruptions in the topsheet while minimizing interruptions in the acquisition layer. An example of DOE is between about 0.080 inches and about 0.300 inches, between about 0.080 inches and about 0.155 inches, or between about 0.080 inches and about 0.135 inches, specifically reciting all 0.001 DOE increments within the specified ranges and all ranges formed therein or thereby.

In general, the non-uniform interruptions in the topsheets herein may introduce fuzz on the wearer-facing surface of the topsheet. However, a certain level of adhesive may reduce the level of fuzz on the surface of the nonwoven material even though there may be a high percentage of broken fibers as a result of the deformation process. Glued dual-layer laminates produced as described herein are evaluated for fuzz. The method utilizes a Martindale Abrasion Tester, based upon ASTM D4966-98. After abrading the samples, they are graded on a scale of 1-10 based on the degree of fiber pilling (1=no fiber pills; 10=large quantity and size of fiber pills). The protrusions are oriented away from the abrader so the land area in between the protrusions is the primary surface abraded. Even though the samples may have a significant amount of fiber breakage (greater than 25%, sometimes greater than 50%) in the side walls of the protrusions, the fuzz value may be low (around 2) for several different material combinations, as long as the layers do not delaminate during abrasion. Delamination is best prevented by glue basis weight, for example a glue basis weight greater than 3 gsm.

### V. Examples

### Prototype

Diaper prototypes were produced using Pampers Premium Protection (size 2) diapers, commercially available in Germany in about December of 2015, by first removing the commercial topsheet, acquisition layer, and distribution layer in these diapers and then inserting the topsheet/acquisition layer laminate (positioned with the discrete integral second regions toward the absorbent core) and the carrier layer/distribution layer laminate.

A hot melt adhesive was applied on a wearer-facing side of the absorbent core (i.e., adhesive placed on the wearer-facing side of the core bag) and the carrier layer/distribution layer laminate was placed with the distribution layer facing the wearer-facing side of the absorbent core, so that an edge of the carrier layer was 40 mm from the diaper chassis's front edge with respect to the longitudinal axis of the diaper and centered with respect to the lateral axis of the diaper.

A hot melt adhesive was applied on the wearer-facing side of carrier layer and the topsheet/acquisition layer laminate was placed on top of it, so that an edge of the acquisition layer was 40mm from the diaper chassis's front edge with respect to a longitudinal axis of the chassis and centered with respect to a lateral axis of the chassis.

The diapers were then compacted in a bag at an In Bag Stack Height, i.e. the total caliper of 10 bi-folded diapers, of 90 mm for 1 week, and the diapers were conditioned for at least 24 hours prior to any testing at 23° C. +/−2° C. and 50% +/−10% Relative Humidity (RH).

The carrier layer of the carrier layer/distribution layer laminate was a hydrophilically coated PP (polypropylene) nonwoven material, composed of two spunlaid and two meltblown layers (SMMS). The basis weight of the carrier layer was 8 gsm. The carrier layer was consolidated and thermopoint-bonded. Then, the carrier layer was coated with a finish made of a mixture of cationic surfactants to render the carrier layer hydrophilic. The carrier layer had a width of 105 mm and a length of 259 mm.

The distribution layer of the carrier layer/distribution layer laminate was composed of intra-fiber crosslinked cellulose fibers and polyacrylic acid was used as cross-linking agent. The distribution layer had a basis weight of 200 gsm. The distribution layer had a width of 80 mm and a length of 239 mm.

The distribution layer and carrier layer were attached to each other with a hot melt adhesive applied in form of spirals with a basis weight of 2.2 gsm. The distribution layer was centered about a lateral axis and a longitudinal axis of the carrier layer.

The topsheet of the topsheet/acquisition layer laminate was different in each example. The topsheet of the topsheet/acquisition layer laminate for each example had a width of 156 mm and a length of 400 mm.

The acquisition layer used was different in each example. The acquisition layer of the topsheet/acquisition layer laminate for each example had a width of 90 mm and a length of 300 mm.

The topsheet and acquisition layer in each example were attached to each other with a hot melt adhesive applied in form of spirals with a basis weight of 5 gsm. The acquisition layer was placed 40 mm from the topsheet' s front edge with respect to a longitudinal axis of the topsheet and centered with respect to a lateral axis of the topsheet.

### Process for Example 1 Topsheet/Acquisition Layer Laminate

The topsheet and acquisition layer of each example were simultaneously mechanically deformed by passing them between a pair of intermeshing male and female rolls (FIG. 21). The discrete integral second regions (e.g., 42) were created such that bases of the discrete integral second regions were present on the topsheet side (i.e., discrete integral second regions extending toward the absorbent core). The teeth on the male roll has a rounded diamond shape like that shown in FIG. 22, with vertical sidewalls and a radiused or rounded edge at the transition between the top and the sidewalls of the tooth. The teeth were 3.38 mm (0.133 inches) long and 2.77 mm (0.109 inches) wide with a CD spacing of 5.08 mm (0.200 inches) and an MD spacing of 8.79 mm (0.346 inches). The recesses in the mating female roll also had a rounded diamond shape, similar to that of the male roll, with a clearance between the rolls of 0.51 mm to 1.09 mm (0.021 inches to 0.043 inches). The process speed was 1.33 meters/second and the depth of engagement (DOE) was 0.135 inches, with the topsheet being in contact with the male roll and the acquisition web of each example being in contact with the female roll.

### Process for Example 2 Topsheet/Acquisition Layer Laminate

The topsheet and acquisition layer of each example were simultaneously mechanically deformed by passing them between a pair of intermeshing male and female rolls (FIG. 21). The discrete integral second regions (e.g., 42) were created such that bases of the discrete integral second regions were present on the topsheet side (i.e., discrete integral second regions extending toward the absorbent core). The teeth on the male roll has a rounded diamond shape like that shown in FIG. 22, with vertical sidewalls and a zero radius or sharp edge at the transition between the top and the sidewalls of the tooth. The teeth were 3.38 mm (0.133 inches) long and 2.77 mm (0.109 inches) wide with a CD spacing of 5.08 mm (0.200 inches) and an MD spacing of 8.79 mm (0.346 inches). The recesses in the mating female roll also had a rounded diamond shape, similar to that of the male roll, with a clearance between the rolls of 0.51 mm to 1.09 mm (0.021 inches to 0.043 inches). The process speed was 1.33 meters/second and the depth of engagement (DOE) was 0.135 inches, with the topsheet being in contact with the male roll and the acquisition web of each example being in contact with the female roll.

### COMPARATIVE EXAMPLE 1

The topsheet of the topsheet/acquisition layer laminate was a hydrophilic coated bi-component PP/PE (polypropylene/polyethylene) nonwoven material, with a PP (polypropylene) core and a PE (polyethylene) sheath, with 2.8 denier fibers. The bi-component PP/PE nonwoven material for the topsheet had an overall basis weight of 20 gsm. The bi-component PP/PE nonwoven material was first coated with a finish made of a fatty acid polyethylene glycol ester for the production of a permanent hydrophilic bi-component PP/PE nonwoven material.

The acquisition layer of the topsheet/acquisition layer laminate was a carded air through bonded material having 4 denier solid round coPET/PET concentric sheath/core bicomponent fibers (i.e. sheath made of polyethylene terephthalate and core made of polyethylene terephthalate). The acquisition layer had an overall basis weight of 65 gsm. The acquisition layer was treated with surfactant to make it hydrophilic.

### EXAMPLE 1

The topsheet of the topsheet/acquisition layer laminate was a hydrophobic mono-component PP (polypropylene) nonwoven material. The PP material for the topsheet had an overall basis weight of 15 gsm and fibers from 1.8 denier to 2.1 denier.

The acquisition layer of the topsheet/acquisition layer laminate was a carded air through bonded material having 4 denier solid round coPET/PET concentric sheath/core bicomponent fibers (i.e. sheath made of polyethylene terephthalate and core made of polyethylene terephthalate). The acquisition layer had an overall basis weight of 65 gsm. The acquisition layer was treated with surfactant to make it hydrophilic.

### COMPARATIVE EXAMPLE 2

The topsheet of the topsheet/acquisition layer laminate was a hydrophilic coated PP monocomponent nonwoven material with 2.8 denier fibers and with a basis weight of 20 gsm by Fitesa of Simpsonville, S.C., U.S.A. Such a material is described in Fitesa's U.S. patent application Ser. No. 14/206,699, entitled “Extensible Nonwoven Fabric”. The nonwoven material was first coated with a finish made of a fatty acid polyethylene glycol ester for the production of a permanent hydrophilic nonwoven material. The topsheet of the topsheet/acquisition layer laminate had a width of 156 mm.

The acquisition layer of the topsheet/acquisition layer laminate was composed of 40% viscose 1.7 dtex, 40% bico 1.7 dtex PE/PP, 20% PET 4.4 dtex. This material was formed of the secondary topsheet of Always® Ultra Normal with Wings, marketed commercially and available in Europe in 2015. The acquisition layer had an overall basis weight of 55 gsm.

### EXAMPLE 2

The topsheet of the topsheet/acquisition layer laminate was a hydrophilic coated PP monocomponent nonwoven material with 2.8 denier fibers and with a basis weight of 20 gsm by Fitesa of Simpsonville, S.C., U.S.A. Such a material is described in Fitesa's U.S. patent application Ser. No. 14/206,699, entitled “Extensible Nonwoven Fabric”. The nonwoven material was first coated with a finish made of a fatty acid polyethylene glycol ester for the production of a permanent hydrophilic nonwoven material. The topsheet of the topsheet/acquisition layer laminate had a width of 156 mm.

The acquisition layer of the topsheet/acquisition layer laminate was composed of 40% viscose 1.7 dtex, 40% bico 1.7 dtex PE/PP, 20% PET 4.4 dtex. This material was a secondary topsheet of Always® Ultra Normal with Wings, marketed commercially in Europe in 2015. The acquisition layer had an overall basis weight of 55 gsm.

### Results of Examples

| | Comparative | | Comparative | |
|---|---|---|---|---|
| Option | Example 1 | Example 1 | Example 2 | Example 2 |
| Rewet - mg | 228 (4) | 96 (14) | 207 (7) | 103 (1) |
| LiTS - g/g | 1.39 (0.01) | 0.18 (0.05) | 1.02 (0.16) | 0.13 (0.01) |
| Gush 1 (sec) | 11.3 (1.0) | 12.3 (1.3) | 13.3 (1.2) | 16.4 (2.0) |
| Gush 2 (sec) | 16.0 (1.6) | 19.8 (3.8) | 27.4 (2.6) | 31.5 (4.1) |
| Gush 3 (sec) | 21.3 (3.3) | 27.3 (6.1) | 39.9 (4.6) | 42.3 (7.4) |
| Gush 4 (sec) | 27.3 (3.5) | 33.0 (5.0) | 49.6 (5.8) | 47.4 (6.2) |
| Total times | 75.9 (8.8) | 92.4 (16.0) | 130.2 (13.1) | 137.6 (18.7) |
| (sec) | | | | |

Additional examples are providing below compared to a planar apertured topsheet of Example 3. Example 3 is a current market product, size 2, Pampers Premium Protection New Baby, available in Germany in about December of 2015.

| Option | Example 1 | Example 2 | Example 3 |
|---|---|---|---|
| TS (topsheet) | PP Spunbond 15 gsm | Mono PP HES | Apertured Bico PP/PE 28 gsm |
| | Hydrophobic | 20 gsm Hydrophobic | Hydrophobic |
| AQL (acquisition | Through Air bonded | Spunlace AQL from | Resin bonded AQL from |
| layer) | AQL 65 gsm | Always Ultra 55 gsm | current size 2 Pampers |
| | | | Premium Protection New Baby |
| | | | 43 gsm |
| Three-dimensional | Yes | Yes | No |
| deformation | | | |
| Gush 1 (sec) | 12.3 (1.3) | 16.4 (2.0) | 13.8 (1.4) |
| Gush 2 (sec) | 19.8 (3.8) | 31.5 (4.1) | 19.5 (1.6) |
| Gush 3 (sec) | 27.3 (6.1) | 42.3 (7.4) | 29 (2.7) |
| Gush 4 (sec) | 33.0 (5.0) | 47.4 (6.2) | 34 (2.7) |
| Total times (sec) | 92.4 (16.0) | 137.6 (18.7) | 96.3 (8.0) |
| Rewet (160 ml) - mg | 96 | 103 | 130 |
| LiTS (160 ml) - g/g | 0.18 | 0.13 | 0.27 |

### VI. Test Methods

### General Sample Preparation

For the Fixed Height Frit Absorption (FHFA) at 20 cm or at 0 cm, a disc sample of the whole topsheet/acquisition layer laminate has to be prepared. For this, the center of the annular sample coincides with center of the topsheet/acquisition layer laminate. The intersection of the longitudinal and transversal axis of the topsheet/acquisition layer laminate defines the center of the topsheet/acquisition layer laminate.

### A. Accelerated Compression Method

1. Cut 10 samples of the specimen to be tested and 11 pieces of a paper towel into a 3 inch×3 inch (7.6 cm×7.6 cm) square.
2. Measure the caliper of each of the 10 specimens at 2.1 kPa and a dwell time of 2 seconds using a Thwing-Albert ProGage Thickness Tester or equivalent with a 50-60 millimeter diameter circular foot. Alternatively, a pressure of 0.5 kPa can be used. Record the pre-compression caliper to the nearest 0.01 mm.
3. Alternate the layers of the specimens to be tested with the pieces of paper towel, starting and ending with the paper towels. The choice of paper towel does not matter and is present to prevent “nesting” of the protrusions in the deformed samples. The samples should be oriented so the edges of each of the specimens and each of the paper towels are relatively aligned, and the protrusions in the specimens are all oriented the same direction.
4. Place the stack of samples into a 40±2° C. oven at 25±3% relative humidity and place a weight on top of the stack. The weight must be larger than the foot of the thickness tester. To simulate high pressures or low in-bag stack heights, apply 35 kPa (e.g. 17.5 kg weight over a 70×70 mm area). To simulate low pressures or high in-bag stack heights, apply 7.0 kPa (e.g. 3.4 kg weight over a 70×70 mm area), 4.0 kPa (e.g., 1.9 kg weight over a 70×70 mm area) of 1.0 kPa (e.g., 0.49 kg weight over a 70×70 mm area).
5. Leave the samples in the oven for 15 hours. After the time period has elapsed, remove the weight from the samples and remove the samples from the oven.
6. Within 30 minutes of removing the samples from the oven, measure the post-compression caliper as directed in step 2 above, making sure to maintain the same order in which the pre-compression caliper was recorded. Record the post-compression caliper of each of the 10 specimens to the nearest 0.01 mm.
7. Let the samples rest at 23±2° C. at 25±3% relative humidity for 24 hours without any weight on them.
8. After 24 hours, measure the post-recovery caliper of each of the 10 specimens as directed in step 2 above, making sure to maintain the same order in which the pre-compression and post-compression calipers were recorded. Record the post-recovery caliper of each of the 10 specimens to the nearest 0.01 mm. Calculate the amount of caliper recovery by subtracting the post-compression caliper from the post-recovery caliper and record to the nearest 0.01 mm.
9. If desired, an average of the 10 specimens can be calculated for the pre-compression, post-compression and post-recovery calipers.

### B. Tensile Method

The MD and CD tensile properties are measured using World Strategic Partners (WSP) (harmonization of the two nonwovens organizations of INDA (North American based) and EDANA (Europe based)) Tensile Method 110.4 (05) Option B, with a 50 mm sample width, 60 mm gauge length, and 60 mm/min rate of extension. Note that the gauge length, rate of extension and resultant strain rate are from different from that specified within the method.

### C. Flat Acquisition Method

This method determines the acquisition times of a baby diaper. The method settings are depending on the diaper size tested. Table 1 shows commonly used diaper size descriptions to be used as reference.

TABLE 1
commonly used size descriptions for diapers

| | |
|---|---|
| Size | Alternative Size Descriptions |

| | | | |
|---|---|---|---|
| 1 | newborn | | |
| 2 | S | P | Infant |
| 3 | M | | Crawler |
| 4 | L | G | Toddler |
| 5 | XL | XG | Walker |
| 6 | XXL | XXG | Junior |

### Apparatus

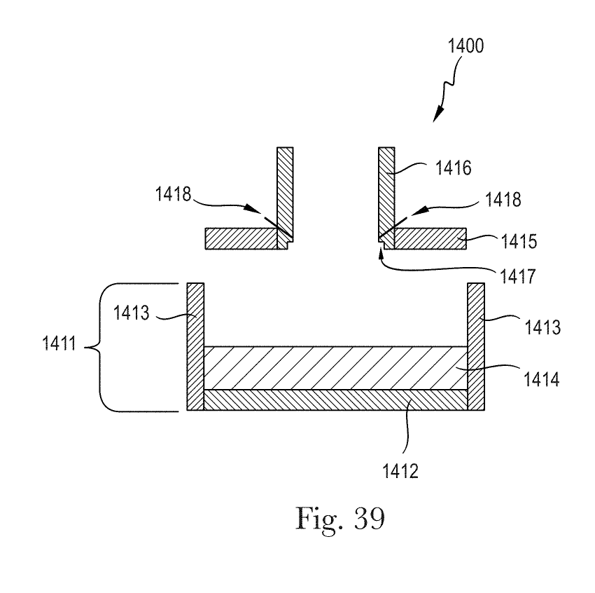

The test apparatus 1400 is shown in FIG. 39 and comprises a trough 1411 made of polycarbonate (e.g. Lexan®) nominally 12.5 mm (0.5 inch) in thickness. The trough 1411 comprises a rectilinear horizontal base 1412 having a length of 508 mm (20.0 inches), and a width of 152 mm (6.0 inches). Two rectilinear vertical sides 1413, 64 mm (2.5 inches) tall×508 mm (20 inches) in length are affixed to the long edges of the base 1412 to form a U-shaped trough 1411 having a length of 508 mm (20.0 inches), an internal width of 152 mm (6.0 inches), and an internal depth of 51 mm (2.0 inches). The front and back ends of the trough 1411 are not enclosed.

A slab of open-cell polyurethane foam 1414 with dimensions 508×152×25 mm is wrapped in polyethylene film and placed in the bottom of the trough 1411 in such a way that the edges of the foam 1414 and the trough 1411 are aligned, and the upper surface of the polyethylene film is smooth and free of seams, wrinkles or imperfections. The polyurethane foam 1414 has a compression hardness at 40% compression CV₄₀ of 2.4 kPa +/−0.4 kPa as determined according to DIN EN ISO 3386 and a density of 16 kg/m3 +/−2 kg/m³ as determined according to DIN EN ISO 845, e.g. a film wrapped foam can be purchased from Crossroads Machine Inc., Englewood Ohio 45322, USA under the description of “FOAM BASE FOR LIQUID ACQUISITION TEST”, or equivalent film-wrapped foam may be used. A reference line is drawn across the width of the upper surface of the polyethylene cover 121 mm (6.0 inches) from one end (the front edge) parallel to the transverse centerline using an indelible marker: such reference line distance must be adjusted according to size based on the table 1.

A rectilinear polycarbonate top plate 1415 has a nominal thickness of 12.5 mm (0.5 inch), a length of 508 mm (20.0 inches), and a width of 146 mm (5.75 inches). A 51 mm (2.0 inch) diameter hole is bored in the center of the top plate 1415 (i.e. the center of the hole is located at the intersection of the longitudinal and transverse axes of the upper surface of the top plate 1415). A polycarbonate cylinder 1416 with an outside diameter of 51 mm (2.0 inches), an internal diameter of 37.5 mm (1.5 inches) and a height of 102 mm (4.0 inches) is glued into the hole in the top plate 1415 so that the bottom edge of the cylinder 1416 is flush with the lower surface of the top plate 1415 and the cylinder 1416 protrudes vertically 89 mm (3.5 inches) above the upper surface of the top plate 1415, and the seam between the cylinder 1416 and the top plate 1415 is watertight. An annular recess 1417 with a height of 2 mm (0.08 inch) and a diameter of 44.5 mm (1.75 inches) is machined into the bottom internal edge of the cylinder 1416. A nylon wire mesh (the opening of this nylon mesh is 1.5 mm, the nylon wire diameter is 0.5 mm) is glued into the recess 1417. The mesh is prepared via cutting a circle of 44.5 mm diameter and cutting of 5 mm of the diameter at each opposite side (i.e. 180° apart). Two 1 mm diameter holes are drilled at a 45° angle to the upper surface of the top plate 1415 so that the holes intersect the inner surface of the cylinder 1416 immediately above the recess 1417 and are at opposite sides of the cylinder 1416 (i.e. 180° apart). Two stainless steel wires 1418 having a diameter of 1 mm are glued into the holes in a watertight fashion so that one end of each wire is flush with the inner cylinder wall and the other end protrudes from the upper surface of the top plate 1415. These wires are referred to as electrodes herein below. A reference line is scribed across the width of the top plate 1415 at a specific distance from the front edge parallel to the transverse centerline. The distance is size specific and shown in table 2 below. For example 121 mm is the distance for size 4. The top plate 1415/cylinder 1416 assembly has a weight of approximately 1180 grams.

TABLE 2
Size specific distances, gush volumes and rates

| | Reference line | | |
|---|---|---|---|
| Size | distance [mm] | Gush volume [ml] | Gush rate [ml/s] |
| 1 | 160 | 24 | 8 |
| 2 | 147 | 40 | 8 |
| 3 | 134 | 50 | 10 |
| 4 | 121 | 75 | 15 |
| 5 | 121 | 75 | 15 |
| 6 | 121 | 75 | 15 |

Two steel weights each weighing 4.5 Kg and measuring 146 mm (5.75 inches) wide, 38 mm (1.5 inches) deep, and approximately 100 mm (4 inches tall) are also required.

### Procedure

All testing is carried out at 23±2° C. and 50±10% relative humidity. The polycarbonate trough 1411 containing the wrapped foam slab 1414 is placed on a suitable flat horizontal surface. A disposable absorbent product is removed from its packaging and the cuff elastics are cut at suitable intervals to allow the product to lay flat. The product is weighed to within ±0.1 grams on a suitable top-loading balance then placed on the covered foam slab 1414 in the acquisition apparatus with the front waist edge of the product aligned with the reference mark on the polyethylene cover. The product is centered along the longitudinal centerline of the apparatus with the topsheet (body-side) of the product facing upwards and the rear waist edge toward the rear end of the foam slab 1414. The top plate 1415 is placed on top of the product with the protruding cylinder facing upwards. The scribed reference line is aligned with the front waist edge of the product and the rear end of the top plate 1415 is aligned with the rear edge of the foam slab 1414. The two 4.5 Kg weights are then gently placed onto the top plate 1415 so that the width of each weight is parallel to the transverse centerline of the top plate, and each weight is 83 mm (3.25 inches) from the front or rear edge of the top plate 1415. The point of the topsheet of the product falling at the center of the cylinder is marked as loading point of the article.

A suitable electrical circuit is connected to the two electrodes to detect the presence of an electrically conductive fluid between them.

A suitable pump; e.g. Model 7520-00 supplied by Cole Parmer Instruments, Chicago, USA, or equivalent; is set up to discharge a 0.9 mass % aqueous solution of sodium chloride through a flexible plastic tube having an internal diameter of 4.8 mm ( 3/16 inch), e.g. Tygon® R-3603 or equivalent. The end portion of the tube is clamped vertically so that it is centered within the cylinder 1416 attached to the top plate 1415 with the discharge end of the tube facing downwards and located 50 mm (2 inches) below the upper edge of the cylinder 1416. The pump is operated via a timer and is pre-calibrated to discharge a gush of 75.0 ml of the 0.9% saline solution at a rate of 15 ml/sec (for size 4 or equivalent). The volume and rate to be used for specific sizes is illustrated in the table 1 above.

In the following the case of size 4 is exemplified: for other sizes the only difference will be to replace the reference line distance, gush volume and gush rate for the specific size as defined in the table 1. The pump is activated and a timer started immediately upon activation. The pump delivers 75 mL of 0.9% NaCl solution to the cylinder 1416 at a rate of 15 ml/sec, then stops. As test fluid is introduced to the cylinder 1416, it typically builds up on top of the absorbent structure to some extent. This fluid completes an electrical circuit between the two electrodes in the cylinder. After the gush has been delivered, the meniscus of the solution drops as the fluid is absorbed into the structure. When the electrical circuit is broken due to the absence of free fluid between the electrodes in the cylinder, the time is noted.

The acquisition time for a particular gush is the time interval between activation of the pump for that gush, and the point at which the electrical circuit is broken.

Four gushes are delivered to the product in this fashion; each gush is 75 ml and is delivered at 15 ml/sec. The time interval between the end of a certain gush, i.e. when the electrical circuit is broken after the liquid acquisition, and the beginning of the next gush is 300 seconds.

The acquisition time for four gushes is recorded to the nearest 1.0 s. Eight products for each option are tested in this fashion and the average gush time for each of the respective gushes (first through fourth) is calculated.

A new foam base 1414 is taken for each test or let the foam base relax for at least 24 hours before re-using it.

The total acquisition time is the sum of the acquisition time of gush 1, the acquisition time of gush 2, the acquisition time of gush 3 and the acquisition time of gush 4. The total acquisition time is expressed in seconds.

### D. Liquid in Topsheet Method

### Objective

The Liquid in topsheet Test Method is the determination of the retained liquid in the topsheet, i.e. a measure of the topsheet dryness. In order to determine the amount of residual fluid in the topsheet, i.e. the liquid in topsheet, it is aimed at measuring the wet topsheet sample weight, i.e. after removing from the diaper test sample and separating from the acquisition web, and dry the topsheet sample weight after at least 16 hours in an oven at 60° C.

### Experiment Setup

Mark the loading point of the diaper as it has been described in the Flat acquisition test method as set out above.
Take the diaper out of the Flat Acquisition Test Method apparatus.
On the diaper, when the topsheet is facing the operator, mark using a permanent ink pen and a plexiglass template (55 mm wide in cross direction, 120 mm long in machine direction, 1-5 mm thick) a rectangle onto the topsheet, symmetrically (centered in cross direction and machine direction) around the loading point.
Perform the Flat acquisition test method as described above.
At least 10 minutes, but not more than 11 minutes after the last gush of the above acquisition test is absorbed, remove the cover plate and weights, and
Place carefully the diaper test sample flat on a lab bench. Preparation of the wet topsheet sample and determination of the Liquid in topsheet

The topsheet/acquisition web laminate is then cut with a scalpel along the marked rectangle.

The wet topsheet of the topsheet/acquisition web laminate is carefully separated from the acquisition web underneath while touching it only with tweezers and as little as possible: if necessary freeze off spray can be used to remove more easily the topsheet without tearing it. The wet topsheet sample has dimensions of 55 mm wide and 120 mm long.

The wet topsheet sample is put in a tarred Petri dish.

Then, the wet topsheet sample is weighed to the nearest 0.001 g, which provides the wet topsheet sample weight.

The wet topsheet sample, contained in its Petri dish, is placed for at least 16 hours into an oven at 60° C.

Then, the Petri dish with the topsheet sample is taken out of the oven; let it cool down to the controlled environment of the test room for at least 10 minutes.

The dry topsheet sample is placed on a new tarred Petri dish. The weight of the dry topsheet sample is recorded from a balance to the nearest 0.001 g.

The liquid in topsheet is then calculated as the difference between the wet topsheet sample and dry topsheet sample weights.

Four samples for each type of absorbent article are tested according to this procedure and the average liquid in topsheet is calculated.

The topsheet load is calculated as the ratio of the liquid in topsheet with the weight of the dry topsheet. Four samples for each type of absorbent article are tested according to this procedure and the average topsheet load is calculated.

### E. Post Acquisition Collagen Rewet Method

This method requires a collagen film having a Fixed Height Frit Absorption (FHFA—0 cm) between 0.48 g/g and 0.66 g/g and FHFA—20 cm between 0.15 g/g and 0.21 g/g as measured according to the method described below. The collagen film has also a basis weight of 31.5 +/−3.5 g/m2. The collagen film can be purchased from Viscofan Group, 31192 Tajonar-Navarra, Spain, under the designation of Naturin COFFI clear, or equivalent material having the characteristics and basis weight as described above.

Before executing the test, the collagen film as is prepared by being cut into circular sheets of 90 mm (3.54 inches) diameter e.g. by using a sample cutter device, and by equilibrating the film in the controlled environment of the test room (see Flat Acquisition Method) for at least 12 hours (tweezers are to be used for all handling of the collagen film).

At least 5 minutes, but not more than 6 minutes after the last gush, which has been performed in the above Flat Acquisition Test Method, is absorbed, the cover plate and weights are removed, and the test sample is carefully placed flat on a lab bench.

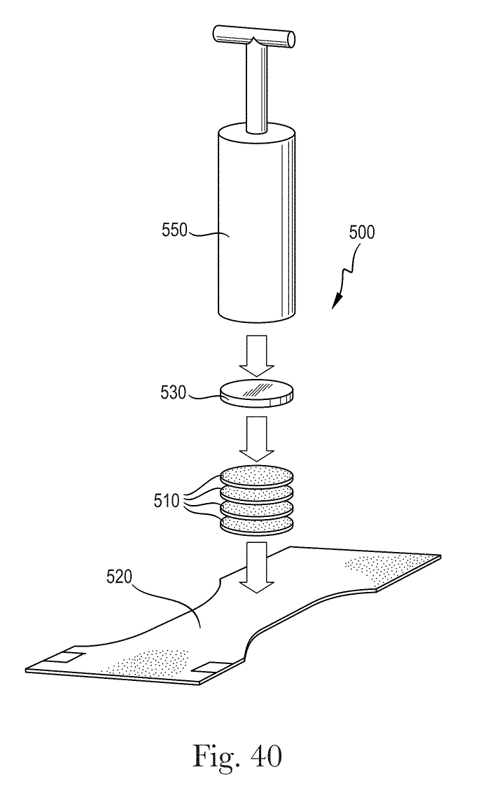

Four sheets of the precut and equilibrated collagen material (510) are weighed with at least one milligram accuracy, and then positioned centered onto the loading point of the article (520), as defined in the Flat Acquisition Method, and covered by a plate (530) made of Poly(methyl methacrylate) (PMMA) (e.g. Perspex®) of 90 mm (3.54 inches) diameter, and about 20 mm (0.78 inches) thickness. A weight (550) of 15 kg is carefully added (also centered). After 30+/−2 seconds the weight and Perspex® plate are carefully removed again, and the collagen films are reweighed (See the system 500 in FIG. 40).

The Rewet result is the moisture pick up of the collagen film, expressed in mg. Four products for each option are tested in this fashion and the average rewet is calculated.

### F. Fixed Height Frit Absorption (FHFA) at 20 cm and at 0 cm Test Methods

This test is suitable of measuring the uptake of a material under the conditions of suction pressures of 20 cm or of 0 cm of fluid, for example of a saline solution (0.9% wt. NaCl solution) after 30 s.

### General Apparatus Setup

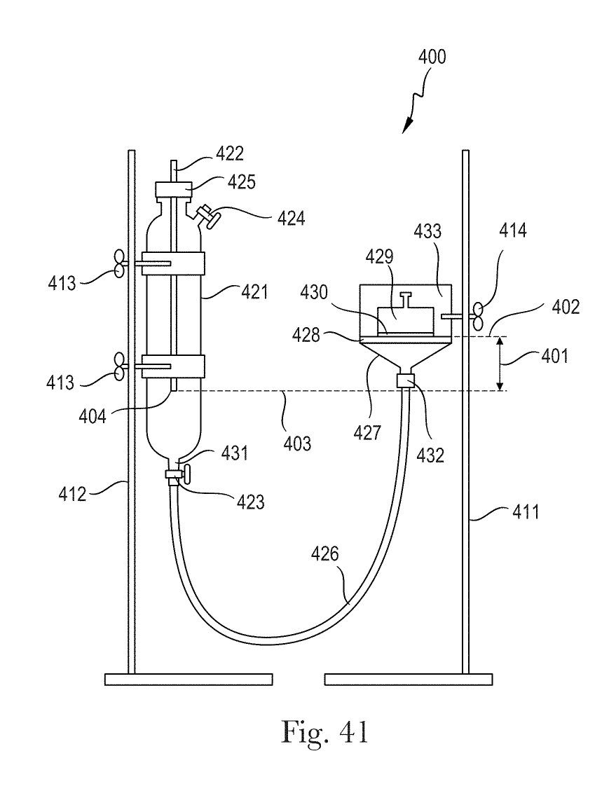

FIG. 41 shows the FHFA measurements setup 400: a suitable fluid delivery reservoir 421, has an air tight stopcock 424 to allow the air release during the filling of the equipment. An open-ended glass tube 422 having an inner diameter of 10 mm extends through a port 425 in the top of the reservoir such that there is an airtight seal between the outside of the tube and the reservoir, this allows maintaining the required zero level of the hydro head during the experiment regardless the amount of liquid in the reservoir. Reservoir 421 is provided with delivery tube 431 having an inlet at the bottom of the reservoir, a stopcock 423, with the outlet connected to the bottom 432 of the sample holder funnel 427 via flexible plastic tubing 426 (e.g. Tygon®). The Fluid reservoir is firmly held in position by means of standard lab clamps 413 and a suitable lab support 412. The internal diameter of the delivery tube 431, stopcock 423, and flexible plastic tubing 426 enables fluid delivery to the sample holder funnel 427 at a high enough flow rate such that such flowrate is higher than the flowrate absorbed by the collagen sample in the conditions of the experiment and exclude that the measured uptake is limited by the fluid flowrate supplied by the equipment system. The reservoir 421 has a capacity of approximately 1 liter. Other fluid delivery systems may be employed provided that they are able to deliver the fluid to the sample holder funnel 427 maintaining the zero level of the hydrostatic liquid pressure 403 at a constant height during the whole experiment.

The sample holder funnel 427 has a bottom connector with an internal diameter of 10 mm, a measurement and a chamber 433 where a glass frit 428 is accommodated. The sample holder chamber has a suitable size to accommodate the sample 430 and the confining pressure weight 429. The frit is sealed to the wall of the chamber 433. The glass frit has pore of specific size of 16-40 μm (glass frit type P 40, as defined by ISO 4793) and a thickness of 7 mm.

The confining pressure weight 429 is a cylinder with a diameter identical to the sample size (6 cm) and a weight of 593.94 g so to apply exactly 2.06 kPa of confining pressure to the sample 430. The sample holder funnel 427 is precisely held in position using a suitable lab support 411 through a standard lab clamp 414. The clamp should allow an easy vertical positioning of the sample holder funnel 427 such that the top of the glass frit 428 can be positioned at a) the same height (+/−1 mm) of the bottom end 404 of the open ended glass tube 422 and b) exactly 20 cm (+/−1 mm) above the bottom end 404 of the open ended glass tube 422. Alternatively two separated clamps are positioned at the abovementioned setups a and b and the sample holder funnel is alternatively moved from one to the other. During the non-usage time, the instrument is kept in proper operating conditions flooding the sample holder funnel 427 with an excess of liquid to guarantee a proper wetting of the glass frit 428 that should be completely below the liquid level. The sample holder funnel 427 is also covered with an air tight cap (not shown) to avoid evaporation and therefore a change in solution salinity. During storage stopcocks 423 and 424 are also accordingly closed to avoid evaporation as well as the open ended tube 422 air tight sealed with a cap (not shown).

### Sample Preparation

During the sample preparation, the sample is only touched with the tweezers. Discs of 6 cm diameter are cut out of the collagen material using any suitable die cutter. The samples are then stored in a closed container, e.g. a petri dish with lid, and conditioned in the controlled environment of the test room for at least 24 hours.

### Material Used

Saline solution at a concentration of 0.9% by weight
FHFA equipment (as set out above)
Bubble level
Analytical balance with a resolution of ±0.001 g with air draft protections.
Funnel
Tweezers
Timer

### Experiment Setup

Before starting the experiment:
   1) The caps to the open ended tube 422 and the sample holder funnel 427 are removed.
   2) Ensuring the stopcock 423 is closed, the stopcock 424 is opened to allow the air to flow out of the liquid reservoir as displaced by liquid during the refilling phase. The liquid reservoir 421 is refilled through top end of the open-end tube 422 with the 0.9% Saline solution with the help of suitable means such a funnel (not shown) at the end of the filling the stopcock 424 is closed.
   If during all the experiments the liquid level would be close to the bottom 404 of the open-ended tube 422, before running the next sample, the liquid reservoir must be refilled repeating this step number 2.
   3) The sample holder funnel 427 is removed from the lab clamp 414 and the excess of liquid is removed pouring it away.
   4) Manually holding the sample holder funnel 427 such that the top of the glass frit 428 lies around 20 cm below the bottom end 404 of the open-ended tube 422 the stop cock 423 is carefully open until the air liquid interface in the open ended tube 422 reaches the bottom end 404 and a few bubble of air escape from tube 422. At this point the stop cock 423 is closed.
   5) The excess of liquid now present in the sample holder funnel 427 is again disposed and the system is now ready to start the measurements.

### For measuring the Fixed Height Frit Absorption (FHFA) at 20 cm, for Each Replicate

1) The sample holder is positioned on the clamp 414 such that the top of the glass frit 428 lies exactly 20 cm (+/−1 mm) above the bottom end 404 of the open-ended tube 422. To ensure a reliable measure it is checked that the glass frit 428 is perfectly horizontal with the help of a bubble level.
2) Any remaining droplets of liquid on top of the glass frit are carefully removed by means of a filter paper of any other suitable material.
3) The sample is weighed with an analytical balance with a resolution of ±0.001 g. The Weight is recorded as Dry Sample Weight (W_{D}) to the nearest 0.001 g when the readings on the balance become constant.
4) 4 sheets of collagen material are carefully aligned on top of each other using tweezers. This stack of 4 sheets of collagen is subsequently referred to as “sample”. The sample 430 is positioned in the center of the sample holder with the help of tweezers with particular care in not altering the orientation and relative position of each of the layers of the acquisition system.
5) The confining weight 429 is positioned centered on the sample
6) The stopcock 423 is opened for 30 +/−1 seconds allowing liquid to flow in the sample and then closed again.
7) The confining weight 429 and the sample 430 are carefully removed from the glass frit 428 with the help of tweezers.
8) The sample 430 is weighed with the analytical balance with a resolution of ±0.001 g. The

Weight is recorded as 20 cm Sample Weight (W₂₀) to the nearest 0.001 g when the readings on the balance become constant.

The measurements of a sample are now completed and a subsequent replicate can be measured repeating the above steps. Once terminated the series of experiment around 1 cm of liquid is added on the Sample Holder funnel 427 to completely submerge the glass frit 428. All the stopcocks are closed and the cap positioned according to the storage condition explained above to avoid evaporation and ensure reliability of the subsequent measurements.

### Calculations

The FHFA at 20 cm (FHFA₂₀) is defined according to the following formula:

FHFA₂₀=(W₂₀−W_{D})/W_{D} and has unit of g/g.

For Measuring the Fixed Height Frit Absorption (FHFA) at 0 cm, for Each Replicate
1) The sample holder is positioned on the clamp 414 such that the top of the glass frit 428 lies exactly 0 cm (+/−1 mm) above the bottom end 404 of the open-ended tube 422. To ensure a reliable measure it is checked that the glass frit 428 is perfectly horizontal with the help of a bubble level.
2) Any remaining droplet of liquid on top of the glass frit are carefully removed by means of a filter paper of any other suitable material.
3) The sample is weighed with an analytical balance with a resolution of ±0.001 g. The Weight is recorded as Dry Sample Weight (W_{D}) to the nearest 0.001 g when the readings on the balance become constant.
4) 4 sheets of collagen material are carefully aligned on top of each other using tweezers. This stack of 4 sheets of collagen is subsequently referred to as “sample”. The sample 430 is positioned in the center of the sample holder with the help of tweezers with particular care in not altering the orientation and relative position of each of the layers of the acquisition system. It is important that the topsheet facing side of each layer is facing now downwards during the experiment in the direction of the glass frit 428, reproducing the liquid flow entrance direction correctly.
5) The confining weight 429 is positioned centered on the sample
6) The stopcock 423 is opened for 30+/−1 seconds allowing liquid to flow in the sample and then closed again.
7) The confining weight 429 and the sample 430 are carefully removed from the glass frit 428 with the help of tweezers.
8) The sample 430 is weighed with the analytical balance with a resolution of ±0.001 g. The Weight is recorded as 0 cm Sample Weight (W₀) to the nearest 0.001 g when the readings on the balance become constant.

The measurements of a sample are now completed and a subsequent replicate can be measured repeating the above steps. Once terminated the series of experiment around 1 cm of liquid is added on the Sample Holder funnel 427 to completely submerge the glass frit 428. All the stopcocks are closed and the cap positioned according to the storage condition explained above to avoid evaporation and ensure reliability of the subsequent measurements.

### Calculations

The FHFA at 0 cm (FHFA0) is defined according to the following formula:

FHFA₀=(W₀−W_{D})/W_{D} and has unit of g/g.

### G. Fiber Diameter and Denier Test

The diameter of fibers in a sample of a web is determined by using a Scanning Electron Microscope (SEM) and image analysis software. A magnification of 500 to 10,000 times is chosen such that the fibers are suitably enlarged for measurement. The samples are sputtered with gold or a palladium compound to avoid electric charging and vibrations of the fibers in the electron beam. A manual procedure for determining the fiber diameters is used. Using a mouse and a cursor tool, the edge of a randomly selected fiber is sought and then measured across its width (i.e., perpendicular to fiber direction at that point) to the other edge of the fiber. A scaled and calibrated image analysis tool provides the scaling to get actual reading in micrometers (μm). Several fibers are thus randomly selected across the sample of the web using the SEM. At least two specimens from the web (or web inside a product) are cut and tested in this manner. Altogether at least 100 such measurements are made and then all data are recorded for statistical analysis. The recorded data are used to calculate average (mean) of the fiber diameters, standard deviation of the fiber diameters, and median of the fiber diameters. Another useful statistic is the calculation of the amount of the population of fibers that is below a certain upper limit. To determine this statistic, the software is programmed to count how many results of the fiber diameters are below an upper limit and that count (divided by total number of data and multiplied by 100%) is reported in percent as percent below the upper limit, such as percent below 1 micrometer diameter or %-submicron, for example.

If the results are to be reported in denier, then the following calculations are made.

Fiber Diameter in denier=Cross-sectional area (in m²)*density (in kg/m³)* 9000 m* 1000 g/kg.

The cross-sectional area is p*diameter²/4. The density for polypropylene, for example, may be taken as 910 kg/m³.

Given the fiber diameter in denier, the physical circular fiber diameter in meters (or micrometers) is calculated from these relationships and vice versa. We denote the measured diameter (in microns) of an individual circular fiber as dᵢ.

In case the fibers have non-circular cross-sections, the measurement of the fiber diameter is determined as and set equal to the hydraulic diameter which is four times the cross-sectional area of the fiber divided by the perimeter of the cross of the fiber (outer perimeter in case of hollow fibers).

### H. Contact Angle Method

Contact angles on substrates are determined using ASTM D7490-13 modified with the specifics as describe herein, using a goniometer and appropriate image analysis software (a suitable instrument is the FTA200, First Ten Angstroms, Portsmouth, Va., or equivalent) fitted with a 1 mL capacity, gas tight syringe with a No. 27 blunt tipped stainless steel needle. Two test fluids are used: Type II reagent water (distilled) in accordance with ASTM Specification D1193-99 and 99+% purity diiodomethane (both available from Sigma Aldrich, St. Louis, Mo.). Contact angles from these two test fluids can further be used to calculate surface energy based on the Owens-Wendt-Kaelble equation. All testing is to be performed at about 23° C.±2 C.° and a relative humidity of about 50%±2%.

A 50 mm by 50 mm nonwoven substrate to be tested is removed from the article taking care to not touch the region of interest or otherwise contaminate the surface during harvesting or subsequent analysis. Condition the samples at about 23° C.±2 C.° and a relative humidity of about 50%±2% for 2 hours prior to testing.

Set up the goniometer on a vibration-isolation table and level the stage according to the manufacturer's instructions. The video capture device must have an acquisition speed capable of capturing at least 10-20 images from the time the drop hits the surface of the specimen to the time it cannot be resolved from the specimen's surface. A capture rate of 900 images/sec is typical. Depending on the hydrophobicity/hydrophilicity of the specimen, the drop may or may not rapidly wet the surface of the nonwoven sample. In the case of slow acquisition, the images should be acquired until 2% of the volume of the drop is absorbed into the specimen. If the acquisition is extremely fast, the first resolved image should be used if the second image shows more than 2% volume loss.

Place the specimen on the goniometer's stage and adjust the hypodermic needle to the distance from the surface recommended by the instrument's manufacturer (typically 3 mm). If necessary adjust the position of the specimen to place the target site under the needle tip. Focus the video device such that a sharp image of the drop on the surface of the specimen can be captured. Start the image acquisition. Deposit a 5 μL±0.1 drop onto the specimen. If there is visible distortion of the drop shape due to movement, repeat at a different, but equivalent, target location. Make two angle measurements on the drop (one on each drop edge) from the image at which there is a 2% drop volume loss. If the contact angles on two edges are different by more than 4°, the values should be excluded and the test repeated at an equivalent location on the specimen. Identify five additional equivalent sites on the specimen and repeat for a total of 6 measurements (12 angles). Calculate the arithmetic mean for this side of the specimen and report to the nearest 0.01°. In like fashion, measure the contact angle on the opposite side of the specimen for 6 drops (12 angles) and report separately to the nearest 0.01°.

To calculate surface energy, the contact angle for both water and diiodomethane must be tested as described above. The value for each test fluid is then substituted into two separate expressions of the Owens-Wendt-Kaelble equation (one for each fluid). This results in two equations and two unknowns, which are then solved for the dispersion and polar components of surface tension.

### The Owens-Wendt-Kaelble Equation

$$\frac{{γ}_{l} ( 1 + cos θ )}{2} = {( {γ}_{l}^{d} + {γ}_{s}^{d} )}^{0.5} + {( {γ}_{l}^{p} + {γ}_{s}^{p} )}^{0.5}$$

where:
θ=the average contact angle for the test liquid on the test specimen
λₗ and λₛ=the surface tension of the test liquid and test specimen, respectively, in dyn/cm
λ^{d} and λ^{p}=the dispersion and polar components of the surface tension, respectively, in dyn/cm

| | Surface Tension | | |
|---|---|---|---|
| | (γₗ) (dyn/cm) | | |
| Solvent | Dispersion | Polar | Total |
| Diiodomethane | 50.8 | 0.0 | 50.8 |
| Water | 21.8 | 51.0 | 72.8 |

The Owens-Wendt-Kaelble equation is simplified to the following when a dispersive solvent such as diiodomethane is used since the polar component is zero:

$$\frac{{γ}_{l} ( 1 + cos θ )}{2} = {( {γ}_{l}^{d} + {γ}_{s}^{d} )}^{0.5}$$

Using the values from the table and θ (measured) for diiodomethane, the equation can be solved for the dispersive component of surface energy (□^{d}ₛ). Now using the values from the table and θ (measured) for water, and the calculated value (□^{d}ₛ), the Owens-Wendt-Kaelble equation can be solved for the polar component of surface energy (□^{p}ₛ). The sum of □^{d}ₛ=□^{p}ₛ is the total solid surface tension and is reported to the nearest 0.1 dyn/cm.

### I. Opening Dimension Test Method

### 1) General Information

The Measured Opening Dimension of the three-dimensional deformation of the topsheet material or topsheet/acquisition layer laminate of an absorbent article are measured using a GFM Primos Optical Profiler instrument commercially available from GFMesstechnik GmbH, Warthestraβe 21, D14513 Teltow/Berlin, Germany. Alternative suitable non-touching surface topology profilers having similar principles of measurement and analysis, can also be used, here GFM Primos is exemplified.

The GFM Primos Optical Profiler instrument includes a compact optical measuring sensor based on a digital micro mirror projection, consisting of the following main components:
a) DMD projector with 800×600 direct digital controlled micro-mirrors
b) CCD camera with high resolution (640×480 pixels)
c) Projection optics adapted to a measuring area of at least 30×40 mm
d) Recording optics adapted to a measuring area of at least 30×40 mm
e) A table tripod based on a small hard stone plate
f) A cold light source (an appropriate unit is the KL 1500 LCD, Schott North America, Inc., Southbridge, Mass.)
g) A measuring, control, and evaluation computer running ODSCAD 6.3 software

Turn on the cold-light source. The settings on the cold-light source are set to provide a color temperature of at least 2800K.

Turn on the computer, monitor, and open the image acquisition/analysis software. In the Primos Optical Profiler instrument, select “Start Measurement” icon from the ODSCAD 6.3 task bar and then click the “Live Image button”.

The instrument is calibrated according to manufacturer's specifications using calibration plates for lateral (X-Y) and vertical (Z). Such Calibration is performed using a rigid solid plate of any non-shiny material having a length of 11 cm, a width of 8 cm and a height of 1 cm. This plate has a groove or machined channel having a rectangular cross-section, a length of 11 cm, a width of 6.000 mm and an exact depth of 2.940 mm. This groove is parallel to the plate length direction. After calibration, the instrument must be able to measure the width and depth dimensions of the groove to within ±0.004 mm.

All testing is performed in a conditioned room maintained at 23±2° C. and 50+/−10% relative humidity. The surface to be measured may be lightly sprayed with a very fine white powder spray. Preferably, the spray is NORD-TEST Developer U 89, available from Helling GmbH, Heidgraben, Germany.

### 2) Opening Dimension Test Method

The topsheet/acquisition layer laminate is extracted from the absorbent article by attaching the absorbent article to a flat surface in a taut planar (i.e. stretched planar) configuration with the topsheet of the topsheet/acquisition layer laminate facing up. Any leg or cuff elastics are severed in order to allow the absorbent article to lie flat. Using scissors, two longitudinal cuts are made through all layers above the absorbent core (i.e. the core wrap) along the edges of the topsheet/acquisition layer laminate. Two transversal cuts are made through the same layers following the front and back waist edges of the absorbent article.

The topsheet/acquisition layer laminate and any other layers above the absorbent core are then removed without perturbing the topsheet/acquisition layer laminate. Freeze spray (e.g. CRC Freeze Spray manufactured by CRC Industries, Inc. 885 Louis Drive, Warminster, Pa. 18974, USA), or equivalent aid may be used to facilitate removal of the uppermost layers from the absorbent article. The topsheet/acquisition layer laminate is then separated from any other layers, including any carrier layer (e.g. a nonwoven carrier layer, a tissue layer), using freeze spray if necessary. If a distribution layer, e.g. a pulp containing layer is attached to the topsheet/acquisition layer laminate, any residual cellulose fibers are carefully removed with tweezers without modifying the acquisition layer.

The topsheet/acquisition layer laminate with three-dimensional protrusions (conditioned at a temperature of 23° C. ±2° C. and a relative humidity of 50%±10% for at least 24 hours) namely “the specimen” is laid down on a hard flat horizontal surface with the body-facing side upward, i.e. the topsheet of the topsheet/acquisition layer laminate being upward. Ensure that the specimen is lying in planar configuration, without being stretched, with the specimen uncovered.

A nominal external pressure of 1.86 kPa (0.27 psi) is then applied to the specimen. Such nominal external pressure is applied without interfering with the topology profile measurement. Such an external pressure is applied using a transparent, non-shining flat Plexiglas® plate 200 mm by 70 mm and appropriate thickness (approximately 5 mm) to achieve a weight of 83 g. The plate is gently placed on top of the specimen, such that the center point of the Plexiglas® plate is at least 40 mm away from any folds, with the entire plate resting on the specimen. A fold corresponds to a part of the absorbent article (e.g. the topsheet/acquisition layer laminate) where the absorbent article has been folded for packaging purposes.

Two 50 mm×70 mm metal weights each having a mass of 1200 g (approximate thickness of 43 mm) are gently placed on the Plexiglas® plate such that a 70 mm edge of each metal weight is aligned with the 70 mm edges of the Plexiglas® plate. A metal frame having external dimensions of 70 mm×80 mm and interior dimensions of 42 mm×61 mm, and a total weight of 142 g (approximate thickness 6 mm), is positioned in the center of the Plexiglas® plate between the two end weights with the longest sides of the frame aligned with the longest sides of the plate.

If the specimen is smaller than 70×200 mm, or if a large enough area without a fold is not present, or if an area of interest is close to the edges of the specimen and can't be analyzed with the Plexiglas and weights settings described above, then the X-Y dimensions of the Plexiglas® plate and the added metal weights may be adjusted to reach a nominal external pressure of 1.86 kPa (0.27 psi) while maintaining a minimum 30×40 mm field of view. At least 10 complete three-dimensional protrusions of the specimen should be captured in the field of view of 30 mm×40 mm.
Position the projection head to be normal to the specimen surface (i.e. to the topsheet of the topsheet/acquisition layer laminate).
Adjust the distance between the specimen and the projection head for best focus.
In the Primos Optical Profiler instrument, turn on the button “Pattern” to make a red cross appear on the screen ross and a black cross appears on the specimen.
Adjust the focus control until the black cross is aligned with the red cross on the screen.
Adjust image brightness then capture a digitized image.
In the Primos Optical Profiler instrument, change the aperture on the lens through the hole in the side of the projector head and/or altering the camera “gain” setting on the screen.
When the illumination is optimum, the red circle at the bottom of the screen labeled “I.O.” will turn green.
Click on the “Measure” button.

The topology of the upper surface of the topsheet/acquisition layer laminate specimen is measured through the Plexiglas plate over the entire field of view 30 mm×40 mm. It is important to keep the specimen still stationary during this time in order to avoid blurring of the captured image. The image should be captured within the 30 seconds following the placement of the Plexiglas plate, metal weights and frame on top of the specimen.

After the image has been captured, the X-Y-Z coordinates of every pixel of the 40 mm×30 mm field of view area are recorded. The X direction is the direction parallel to the longest edge of the rectangular field of view, the Y direction is the direction parallel to the shortest edge of the rectangular field of view. The Z direction is the direction perpendicular to the X-Y plane. The X-Y plane is horizontal while the Z direction is vertical, i.e. orthogonal to the X-Y plane.

These data are smoothed and filtered using a polynomial filter (n=6), a median filter 11 pixels by 11 pixels, and a structure filter 81 pixels by 81 pixels. The polynomial filter (n=6) approximates the X-Y-Z coordinate surface with a polynomial of order 6 and returns the difference to the approximated polynomial. The median filter 11 pixels by 11 pixels divides the field of view (40 mm×30 mm) in X-Y squares of 11 pixels by 11 pixels. The Z coordinate of the pixel located at the center of a given 11 pixels by 11 pixels square will be replaced by the mean Z value of all the pixels of this given square. The structure filter 81 pixels by 81 pixels, removes the waviness of the structure and translates all the Z peak values belonging to the bottom surface of the Plexiglas plate to a top X-Y plane.

A Reference Plane is then defined as the X-Y plane intercepting the surface topology profile of the entire field of view (i.e. 30 mm×40 mm), 100 microns below this top X-Y plane. In the Primos Optical Profiler instrument, to measure the Material Area of the Reference Plane (Z=−0.1 mm), click on the button “Evaluate”. Then, apply a pre-filtering routine including a polynomial filter (n=6), a median filter 11 by 11 and a structure filter (n=81) using the function “Filter”. Save the image to a computer file with “.omc” extension.

The Empty Area of the reference plane can be defined as the area of the Reference Plane that is above the surface profile. The Empty Areas having boundaries strictly located inside the field of view area (i.e. 30 mm×40 mm) without crossing or overlapping with the boundaries of the field of view area (i.e. 40 mm×30 mm) are defined as Isolated Empty Area(s). The Measured Opening dimension is defined for an Isolated Empty Area as the diameter of the biggest circle that can be inscribed inside a given Isolated Empty Area. This circle should only overlap with the Isolated Empty Area.

In the Primos Optical Profiler instrument, this can be done by clicking on “Draw circle” and drawing the biggest inscribed circle possible in a chosen Isolated Empty Area. Click on “Show sectional picture”, the circle diameter can be measure via clicking on the extremity of the sectional picture profile and then clicking on “Horizontal distance” to obtain the Opening dimension.

For the acquired and digitized image, the Opening dimension of all the Isolated Empty Areas is determined. Then, the Measured Opening dimension is calculated as the arithmetic average of the 6 biggest Opening dimensions.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “90°” is intended to mean “about 90°”.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All documents cited in the Detailed Description are, in relevant part, incorporated herein by reference; the citation of any document is not to be construed as an admission that it is prior art with respect to the present disclosure. To the extent that any meaning or definition of a term in this written document conflicts with any meaning or definition of the term in a document incorporated by reference, the meaning or definition assigned to the term in this written document shall govern.

While particular forms of the present disclosure have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a laminate;
a backsheet; and
an absorbent core disposed at least partially between the laminate and the backsheet;
wherein the laminate comprises:
a non-apertured topsheet; and
a nonwoven, non-apertured acquisition layer, wherein the topsheet is less hydrophilic than the acquisition layer;
a generally planar first region; and
a plurality of discrete integral second regions formed in the topsheet and the acquisition layer to nest portions of the topsheet and the acquisition layer, wherein the plurality of discrete integral second regions extend towards the absorbent core, wherein the plurality of discrete integral second regions each comprise a wearer-facing surface, wherein a non-uniform interruption is defined in the topsheet in each of at least some of the plurality of discrete integral second regions, and wherein portions of the acquisition layer form portions of the wearer-facing surface in the non-uniform interruption in the at least some of the discrete integral second regions.

2. The absorbent article of claim 1, wherein the topsheet comprises a nonwoven material.

3. The absorbent article of claim 2, wherein the nonwoven material is hydrophobic.

4. The absorbent article of claim 1, wherein the topsheet is a hydrophobic film.

5. The absorbent article of claim 1, wherein the topsheet is hydrophobic, and wherein the acquisition layer is hydrophilic.

6. The absorbent article of claim 1, wherein a second non-uniform interruption is defined in the topsheet in the at least some of the plurality of discrete integral second regions.

7. The absorbent article of claim 1, wherein the non-uniform interruption is not a predetermined aperture.

8. The absorbent article of claim 1, wherein the non-uniform interruption is defined in a side wall of the at least some of the discrete integral second regions.

9. The absorbent article of claim 1, wherein the non-uniform interruption is defined in a distal end of the at least some of the discrete integral second regions.

10. The absorbent article of claim 1, wherein fibers of the topsheet have a smaller average diameter than an average diameter of fibers of the acquisition layer.

11. The absorbent article of claim 1, wherein the topsheet comprises cotton.

12. The absorbent article of claim 11, wherein the topsheet is hydrophobic or comprises hydrophobic cotton.

13. An absorbent article comprising:
a laminate;
a backsheet; and
an absorbent core disposed at least partially between the laminate and the backsheet;
wherein the laminate comprises:
a nonwoven, non-apertured topsheet; and
a nonwoven, non-apertured acquisition layer, wherein the topsheet is less hydrophilic than the acquisition layer;
a generally planar continuous land area; and
a plurality of three-dimensional deformations, wherein at least some of the plurality of three-dimensional deformations are surrounded by at least some of the generally planar continuous land area, wherein the plurality of three-dimensional deformations extend towards the absorbent core, wherein the plurality of three-dimensional deformations each comprise a wearer-facing surface, wherein a non-uniform interruption is defined in the topsheet in each of at least some of the plurality of three-dimensional deformations, and wherein portions of the acquisition layer form portions of the wearer-facing surface in the non-uniform interruption in the at least some of the plurality of three-dimensional deformations.

14. The absorbent article of claim 13, wherein the topsheet is hydrophobic, and wherein the acquisition layer is hydrophilic.

15. The absorbent article of claim 13, wherein a second non-uniform interruption is defined in the topsheet in the at least some of the plurality of three-dimensional deformations, and wherein second portions of the acquisition layer form portions of the wearer-facing surface in the second non-uniform interruption in the at least some of the plurality of three-dimensional deformations.

16. The absorbent article of claim 13, wherein the non-uniform interruption is not a predetermined aperture.

17. The absorbent article of claim 13, wherein the at least some of the three-dimensional deformations comprise a conical portion on the wearer-facing surface intermediate a portion of the generally planar continuous land area and the non-uniform interruption.

18. The absorbent article of claim 13, wherein the non-uniform interruption is defined in a side wall of the at least some three-dimensional deformations.

19. The absorbent article of claim 13, wherein the non-uniform interruption is defined in a distal end of the at least some three-dimensional deformations.

20. The absorbent article of claim 13, wherein fibers of the topsheet have a smaller average diameter than an average diameter of fibers of the acquisition layer.
